(19) United States
(12) Patent Application Publication
(10) Pub. No.: US 2025/0163489 A1
(43) Pub. Date: May 22, 2025

(54) ENZYMES AND USES THEREOF

(71) Applicants: University of Newcastle upon Tyne, Newcastle upon Tyne (GB); The University of Birmingham, Birmingham (GB)

(72) Inventors: Elisabeth LOWE, Newcastle upon Tyne (GB); Patrick MOYNIHAN, Birmingham (GB); Joseph MANION, Newcastle upon Tyne (GB); Amar GUDKA, Birmingham (GB)

(21) Appl. No.: 18/842,674

(22) PCT Filed: Mar. 1, 2023
(86) PCT No.: PCT/GB2023/050457

§ 371 Date: Aug. 29, 2024

(30) Foreign Application Priority Data
Mar. 2, 2022 (GB) 2202916.9

(51) Int. Cl. C12Q 1/04; C12Q 1/34

(52) U.S. Cl. CPC C12Q 1/04; C12Q 1/34; G01N 2333/35; G01N 2333/924

(57) ABSTRACT

The present invention provides uses of enzymes for cleaving a linkage between two D-galactofuranoses and/or two D-arabinofuranoses in a polysaccharide. An example of a polysaccharide where such linkages are present is arabinogalactan. The invention also provides compositions comprising said enzymes, as well as methods for preparing a sample, and methods for determining the presence of a bacterium of the Actinomycetota phylum in a sample using said enzymes. Additionally, the invention provides pharmaceutical formulations comprising a composition of the invention, as well as uses of the pharmaceutical formulations as a medicament, for example to treat a bacterial infection caused by a bacterium of the Actinomycetota phylum.

### CROSS-REFERENCE TO RELATED APPLICATIONS

This application is a US National Stage Entry of PCT Application No. PCT/GB2023/050457, filed on Mar. 1, 2023, which is an International Application that claims priority from a GB Patent Application No. 2202916.9, filed on Mar. 2, 2022, the contents of each of the above applications are incorporated herein by reference in their entireties.

### FIELD OF INVENTION

The present invention provides uses of enzymes for cleaving a linkage between two D-galactofuranoses and/or two D-arabinofuranoses in a polysaccharide. An example of a polysaccharide where such linkages are present is arabinogalactan. The invention also provides compositions comprising said enzymes, as well as methods for preparing a sample, and methods for determining the presence of a bacterium of the Actinomycetota phylum in a sample using said enzymes. Additionally, the invention provides pharmaceutical formulations comprising a composition of the invention, as well as uses of the pharmaceutical formulations as a medicament, for example to treat a bacterial infection caused by a bacterium of the Actinomycetota phylum.

### BACKGROUND

Pulmonary tuberculosis (TB) continues to cause ill health and deaths across many populations, especially in poorly developed areas of the world. In 2018 there was an estimated 9-1.1 million cases of TB worldwide. Increasing access to early and accurate diagnosis using a WHO-recommended rapid diagnostic (WRD) is one of the main components of TB laboratory strengthening efforts under the “End TB Strategy”. Although many tests and diagnostic strategies already exist, such as chest x-rays, smear microscopy, the Xpert MTB/RIF assay and Loopamp MTBC Detection Kit, there remains a need for accurate, affordable, point-of-care TB tests, with no requirement for electricity or specialised laboratories, that can be easily performed by a healthcare professional.

One of the main problems associated with developing such tests is related to the complexity of sputum specimens in which mycobacteria can be detected and from which mycobacterial DNA can be extracted. The majority of DNA extraction systems for sputum samples have separate offboard liquefaction and/or lysis steps resulting in only semi-automated systems that require more specimen manipulation by the end user when compared to a full walk away (automated) assay, and additional equipment.

In addition, DNA from mycobacteria is difficult to extract due to their complex cell wall and an outer waxy coat. The mycobacterial cell wall consists of three layers, each conserved amongst mycobacteria and other closely related organisms. Like other bacteria, peptidoglycan forms the basal layer of the cell wall. At the other extremity are mycolic acids which give the organisms their characteristic waxy appearance and are interspersed with a host of species-specific lipids. Joining these two layers covalently is a complex polysaccharide called arabinogalactan (AG), which has a chemical composition unique to this group of organisms and entirely distinct from the similarly named molecule found in plants.

AG is comprised of two domains with a β-D-galactofuranose backbone decorated by large α-D-arabinofuranose branches. The structure and biosynthesis of this molecule has undergone intense scrutiny, due in part to it being a target of the potent antimycobacterial drug, ethambutol. Ethambutol targets the glycosyltransferase family C proteins in the cell envelope of mycobacteria, which collectively are responsible for polymerisation and decoration of the polysaccharide. Similarly, the biogenesis of mycolic acids and peptidoglycan have undergone intense scrutiny due to their essentiality, and their synthesis being the target of isoniazid and β-lactams respectively. Despite these advances, very few enzymes have been identified that can degrade arabinogalactan, and none have been reported to have lytic potential.

The lysis of mycobacteria to-date has been dominated by mechanical techniques which result in the physical shearing of the wall (bead beading, sonication) or pyrolysis which uses heat to bring about cleavage of the wall. The development of lytic enzymes for mycobacteria has been much slower, with research focused mainly on phage lysins.

The present invention provides novel enzymes for degrading arabinogalactan and aiding in the lysis of mycobacteria. Thereby, the present invention aims to at least partially ameliorate some of the problems with mycobacterial DNA extraction and accurate and rapid TB diagnosis.

### SUMMARY OF INVENTION

The present invention is based on the inventors' identification of new enzymes useful for lysing bacteria of the Actinomycetota phylum, especially mycobacteria. Whilst enzymes capable of degrading the peptidoglycan and mycolic acid components of the mycobacterial cell wall have been identified, there remains a need for enzymes capable of degrading the arabinogalactan component of the bacterial cell wall, in order to allow easy cell lysis and access to specific biomarkers, such as DNA.

The present invention, at least partially, aims to fulfil this need by providing enzymes that have the ability to degrade the galactan portion of arabinogalactan by cleaving a linkage between two D-galactofuranose molecules, and/or degrade the arabinan portion of arabinogalactan by cleaving a linkage between two D-arabinofuranoses. These newly identified enzymes comprise amino acid sequences as shown in SEQ ID NO: 1 to SEQ ID NO: 14.

As explained in more detail in the Examples section below, the present inventors have identified these enzymes by analysing organisms isolated from the human gut microbiome for their ability to survive on mycobacterial arabinogalactan as a sole-carbon source. The specific enzymes these bacteria used to achieve this were then identified using-omics approaches.

Surprisingly, the inventors found that although these enzymes share functional similarity (i.e. they are able to degrade components of arabinogalactan), many of the enzymes share little or substantially no amino acid sequence homology. Therefore, predicting the function of these enzymes solely on the basis of their amino acid sequences is not possible, and can only be determined empirically, through complex and labour intensive experimental methods.

Accordingly, in one aspect, the present invention provides use of an enzyme for cleaving a linkage between two D-galactofuranoses in a polysaccharide, wherein the enzyme comprises an amino acid sequence having at least 70% sequence identity to a sequence provided in Table 1.

Suitably, the enzyme may be an exo-D-galactofuranosidase comprising an amino acid sequence as shown in SEQ ID NO: 1 or SEQ ID NO: 2; or an amino acid sequence which has at least 70% sequence identity thereto.

Suitably, the exo-D-galactofuranosidase may comprise an amino acid sequence as shown in SEQ ID NO: 1 or SEQ ID NO: 2.

Suitably, the enzyme may be an endo-D-galactofuranase comprising an amino acid sequence as shown in SEQ ID NO: 3; or an amino acid sequence which has at least 70% sequence identity thereto.

Suitably, the endo-D-galactofuranase may comprise an amino acid sequence as shown in SEQ ID NO: 3.

Suitably, when the enzyme is for cleaving a linkage between two D-galactofuranoses, the polysaccharide may be a D-galactofuranose polysaccharide.

In one aspect, the present invention provides use of an enzyme for cleaving a linkage between two D-arabinofuranoses in a polysaccharide, wherein the enzyme comprises an amino acid sequence having at least 70% sequence identity to a sequence provided in Table 2.

Suitably, the enzyme may be an endo-D-arabinofuranase comprising an amino acid sequence as shown in SEQ ID NO: 4, SEQ ID NO: 5, SEQ ID NO: 6, SEQ ID NO: 7, SEQ ID NO: 8, or SEQ ID NO: 9; or an amino acid sequence which has at least 70% sequence identity thereto.

Suitably, the endo-D-arabinofuranase may comprise an amino acid sequence as shown in SEQ ID NO: 4, SEQ ID NO: 5 SEQ ID NO: 6, SEQ ID NO: 7, SEQ ID NO: 8, or SEQ ID NO: 9.

Suitably, the enzyme may be an exo-D-arabinofuranosidase comprising an amino acid sequence as shown in SEQ ID NO: 10, SEQ ID NO: 11, SEQ ID NO: 12, SEQ ID NO: 13, or SEQ ID NO: 14; or an amino acid sequence which has at least 70% sequence identity thereto.

Suitably, the exo-D-arabinofuranosidase may comprise an amino acid sequence as shown in SEQ ID NO: 10, SEQ ID NO: 11, SEQ ID NO: 12, SEQ ID NO: 13, or SEQ ID NO: 14.

Suitably, when the enzyme is for cleaving a linkage between two D-arabinofuranoses, the polysaccharide may be lipoarabinomannan (LAM) or pilin oligosaccharide.

Suitably, the polysaccharide may be an arabinogalactan.

Suitably, the polysaccharide may be a bacterial cell wall polysaccharide.

Suitably, the bacterial cell wall polysaccharide may be from a bacterium of the Actinomycetota phylum.

Suitably, the bacterium may be of the Mycobacteriales order.

Suitably, the bacterium may be of the family selected from the group consisting of: Segniliparaceae, Mycobacteriaceae, Nocardiaceae, Tsukamurellaceae, Gordoniaceae, Lawsonellaceae, Corynebacteriaceae and Dietziaceae.

Suitably, the bacterium may be of the family Mycobacteriaceae.

Suitably, the bacterium may be selected from the group consisting of M. tuberculosis, M. bovis, M. africanum, M. canetti, M. microti. M. smegmatis, M. fortuitum, M. marinum, M. ulcerans, M. paratuberculosis, M. celatum, M. avium, M. leprae, M. lepraemurium, M. intracellulare M. scrofulaceum, M. xenopi, M. genavense, M. kansasii, M. simiae, M. szulgai, M. haemophilus, M. asiaticum, M. malmoense, M. vaccae, M. caprae, M. pinnipe dii and M. shimoidei.

In one aspect, the present invention provides a composition for degrading a Actinomycetota bacterial cell wall polysaccharide, wherein the composition comprises at least two bacterial cell wall degrading enzymes, wherein at least one of the at least two cell wall degrading enzymes is an enzyme comprising an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or an amino acid sequence which has at least 70% sequence identity thereto.

Suitably, the composition may comprise:
a) at least one enzyme comprising an amino acid sequence as shown in SEQ ID NO: 1, SEQ ID NO: 2, or SEQ ID NO: 3; or an amino acid sequence which has at least 70% sequence identity thereto; and
b) at least one enzyme comprising an amino acid sequence as shown in SEQ ID NO: 4, SEQ ID NO: 5, SEQ ID NO: 6, SEQ ID NO: 10, SEQ ID NO: 11 SEQ ID NO: 12, or SEQ ID NO: 13; or an amino acid sequence which has at least 70% sequence identity thereto.

Suitably, the composition may comprise an exo-D-galactofuranosidase comprising an amino acid sequence as shown in SEQ ID NO: 1 or SEQ ID NO: 2; or an amino acid sequence which has at least 70% sequence identity thereto.

Suitably, the composition may comprise an endo-D-galactofuranase comprising an amino acid sequence as shown in SEQ ID NO: 3; or an amino acid sequence which has at least 70% sequence identity thereto.

Suitably, the composition may comprise an endo-D-arabinofuranase comprising an amino acid sequence as shown in SEQ ID NO: 4, SEQ ID NO: 5 or SEQ ID NO: 6; or an amino acid sequence which has at least 70% sequence identity thereto.

Suitably, the composition may comprise an exo-D-arabinofuranosidase comprising an amino acid sequence as shown in SEQ ID NO: 10, SEQ ID NO: 11, SEQ ID NO: 12, or SEQ ID NO: 13; or an amino acid sequence which has at least 70% sequence identity thereto.

Suitably, the composition may comprise:
(i) an exo-D-galactofuranosidase comprising an amino acid sequence as shown in SEQ ID NO: 1 or SEQ ID NO: 2; or an amino acid sequence which has at least 70% sequence identity thereto;
(ii) an endo-D-galactofuranase comprising an amino acid sequence as shown in SEQ ID NO: 3; or an amino acid sequence which has at least 70% sequence identity thereto; and
(iii) an endo-D-arabinofuranase comprising an amino acid sequence as shown in SEQ ID NO: 4, SEQ ID NO: 5 or SEQ ID NO:6; or an amino acid sequence which has at least 70% sequence identity thereto.

Suitably, the composition may further comprise an exo-D-arabinofuranosidase comprising an amino acid sequence as shown in SEQ ID NO: 10, SEQ ID NO: 11, SEQ ID NO: 12, or SEQ ID NO: 13; or an amino acid sequence which has at least 70% sequence identity thereto.

Suitably, the composition may comprise an:
(i) an exo-D-galactofuranosidase comprising an amino acid sequence as shown in SEQ ID NO: 1 and/or SEQ ID NO: 2; or an amino acid sequence which has at least 70% sequence identity thereto;
(ii) an endo-D-galactofuranase comprising an amino acid sequence as shown in SEQ ID NO: 3; or an amino acid sequence which has at least 70% sequence identity thereto;
(iii) an endo-D-arabinofuranase comprising an amino acid sequence as shown in SEQ ID NO: 5 and/or SEQ ID NO: 6; or an amino acid sequence which has at least 70% sequence identity thereto; and
(iv) an exo-D-arabinofuranosidase comprising an amino acid sequence as shown in SEQ ID NO: 11 and/or SEQ ID NO: 13; or an amino acid sequence which has at least 70% sequence identity thereto.

Suitably, the composition may comprise:
(i) an exo-D-galactofuranosidase comprising an amino acid sequence as shown in SEQ ID NO: 1 and/or SEQ ID NO: 2;
(ii) an endo-D-galactofuranase comprising an amino acid sequence as shown in SEQ ID NO: 3;
(iii) an endo-D-arabinofuranase comprising an amino acid sequence as shown in SEQ ID NO: 5 and/or SEQ ID NO: 6; and
(iv) an exo-D-arabinofuranosidase comprising an amino acid sequence as shown in SEQ ID NO: 11 and/or SEQ ID NO: 13.

Suitably, the composition may comprise an:
(i) an exo-D-galactofuranosidase comprising an amino acid sequence as shown in SEQ ID NO: 1;
(ii) an endo-D-galactofuranase comprising an amino acid sequence as shown in SEQ ID NO: 3;
(iii) an endo-D-arabinofuranase comprising an amino acid sequence as shown in SEQ ID NO: 5 and SEQ ID NO: 6; and
(iv) an exo-D-arabinofuranosidase comprising an amino acid sequence as shown in SEQ ID NO: 11 and SEQ ID NO: 13.

Suitably, the composition may further comprise an exo-D-arabinofuranosidase comprising an amino acid sequence as shown in SEQ ID NO: 11 and/or SEQ ID NO: 13; or an amino acid sequence which has at least 70% sequence identity thereto.

Suitably, the composition may comprise:
(i) an exo-D-galactofuranosidase comprising an amino acid sequence as shown in SEQ ID NO: 1; or an amino acid sequence which has at least 70% sequence identity thereto;
(ii) an endo-D-galactofuranase comprising an amino acid sequence as shown in SEQ ID NO: 3; or an amino acid sequence which has at least 70% sequence identity thereto;
(iii) an endo-D-arabinofuranase comprising an amino acid sequence as shown in SEQ ID NO: 6; or an amino acid sequence which has at least 70% sequence identity thereto; and
(iv) an exo-D-arabinofuranosidase comprising an amino acid sequence as shown in SEQ ID NO: 11 and/or SEQ ID NO: 13; or an amino acid sequence which has at least 70% sequence identity thereto.

Suitably, the composition may comprise:
(i) an exo-D-galactofuranosidase comprising an amino acid sequence as shown in SEQ ID NO: 1;
(ii) an endo-D-galactofuranase comprising an amino acid sequence as shown in SEQ ID NO: 3;
(iii) an endo-D-arabinofuranase comprising an amino acid sequence as shown in SEQ ID NO: 6; and
(iv) an exo-D-arabinofuranosidase comprising an amino acid sequence as shown in SEQ ID NO: 11 and SEQ ID NO: 13.

Suitably, the composition may be freeze dried.

Suitably, the second of the at least two cell wall degrading enzymes may be selected from the group consisting of a peptidoglycan degrading enzyme, a mycolic acid degrading enzyme, a lipoarabinomannan degrading enzyme, and a pilin oligosaccharide degrading enzyme.

Suitably, the mycolic acid degrading enzyme may be a lysin B or a Candida rugosa lipase.

Suitably, the peptidoglycan degrading enzyme may be a lysozyme, optionally a hen-egg white lysozyme.

In a further aspect, the present invention provides use of a composition of the invention for cleaving a linkage between two D-galactofuranoses and/or cleaving a linkage between two D-arabinofuranoses in a polysaccharide.

In a further aspect, the present invention provides a method for preparing a sample for the detection of a bacterium of the Actinomycetota phylum, the method comprising contacting the sample with a composition of the invention under conditions that allow for degradation of Actinomycetota bacterial cell wall polysaccharides to occur.

Suitably, the method lyses the bacteria in the sample.

In a further aspect, the present invention provides a method of determining the presence of a bacterium of the Actinomycetota phylum in a sample, comprising:
(i) providing a sample prepared according to the method for preparing a sample; and
(ii) detecting a biomarker indicative of the presence of the bacterium in the sample.

Suitably, the biomarker may be a nucleic acid, protein, carbohydrate, and/or a lipid.

Suitably, the sample may be selected from the group consisting of a sputum sample, a blood sample, a stool sample, and a urine sample.

Suitably, the bacterium may be of the Mycobacteriales order.

Suitably, the bacterium may be of the family selected from the group consisting of: Segniliparaceae, Mycobacteriaceae, Nocardiaceae, Tsukamurellaceae, Gordoniaceae, Lawsonellaceae, Corynebacteriaceae and Dietziaceae.

Suitably, the bacterium may be of the family Mycobacteriaceae, optionally wherein the bacterium is M. tuberculosis.

In a further aspect, the present invention provides a pharmaceutical formulation comprising a composition of the invention, and a pharmaceutically acceptable excipient carrier, adjuvant, and/or diluent.

Suitably, the formulation may be for pulmonary delivery and/or topical administration.

In a further aspect, the present invention provides a pharmaceutical formulation of the invention for use as a medicament.

In a further aspect, the present invention provides a pharmaceutical formulation of the invention for use in treating a bacterial infection caused by a bacterium of the Actinomycetota phylum.

In a further aspect, the present invention provides a method of treating a bacterial infection caused by bacterium of the Actinomycetota phylum in a subject, comprising administering a therapeutically effective amount of the pharmaceutical formulation of the invention to a subject in need thereof.

Suitably, the infection may be a mycobacterial infection.

It will be appreciated that except where the context requires otherwise, embodiments described in respect of one aspect of the invention will also be applicable to the other aspects of the invention. Thus, except for where the context requires otherwise, the considerations set out in this disclosure should be considered to be applicable to the uses, compositions, formulations and methods of the invention.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE DRAWINGS

Embodiments of the invention are further described hereinafter with reference to the accompanying drawings, in which:

FIG. 1 is a schematic of the mycobacterial cell wall. The mycobacterial cell wall is a complex structure whose three main components are peptidoglycan, arabinogalactan and mycolic acids. There are many enzyme classes known to cleave peptidoglycan, including mutanolysin. In contrast, only a single enzyme which cleaves mycolic acids has been described, LysB. Enzymes which cleave within the D-arabinan chains of arabinogalactan have not been characterised, and only one enzyme which cleaves the galactan component has been identified, albeit with weak activity.

FIG. 2 is a diagram showing precise structure of D-arabinan containing glycopolymers in the mycobacterial cell wall as currently understood. Lipoarabinomannan (LAM) and arabinogalactan (AG) both contain D-arabinan motifs within their structures. In LAM this is connected to a mannosyl structure and attached to a lipid anchor. AG in contrast is anchored to the peptidoglycan (PG) via a conserved linker unit.

FIG. 3 depicts growth of bacteria on mycobacterial AG. Left panel-bacterial species grown on AG as determined by an increase in OD and (right) the HPAEC-PAD traces from supernatants of these growths, showing release of galactose by B. finegoldii and arabinose and galactose by D. gadei. Proteomics/transcriptomics were then used to identify the genes/proteins upregulated during growth on galactan or arabinan.

FIG. 4 shows Bacteroides finegoldii PULs with galactanase enzymes. Known GH families are coloured dark grey, SusCD transporters are dotted and regulators are chequered. New GH family GalF is diagonally striped. Unknown proteins are light grey.

FIG. 5 shows galactanase activity of B. finegoldii enzymes. a) BACFIN_04787 and b) BACFIN_08810 incubated with 2 mg/ml C. glutamicum D-galactan in 20 mM Tris pH 8.0, 100 mM NaCl, for 0-30 mins at 37° C., reaction stopped by boiling at the indicated time points and analysed by TLC, stained with orcinol. c) Overnight incubations of individual enzymes in 20 mM HEPES pH 7.5, 120 mM NaCl (1 μM) with 2 mg/ml D-galactan.

FIG. 6 shows D. gadei AG utilisation PUL. Schematic of D-arabinan degrading PUL identified in Dysgonomonas gadei. Genes with annotated/predicted functions are mid-grey, except SusC and SusD glycan transporters which are dotted. Light grey genes have not yet been studied. Genes with horizontal stripes, diagonal stripes or cross-hatched are the three novel families which encode proteins which are able to degrade D-arabinan.

FIG. 7 shows Dysgonomonas gadei enzymes have D-arabinanase activity. Enzymes 02480 (80) and 02481 (81) incubated with 2 mg·ml⁻¹ M. smegmatis arabinogalactan (AG). Left, TLC showing release of oligosaccharides and right, HPAEC-PAD analysis of the oligosaccharide fraction with monosaccharides inset.

FIG. 8 shows activity of M. tuberculosis endo-D-arabinanase enzyme. a) Rv3707c was incubated with D-arabinan glycosylated pili for 24 h and the oligomeric D-arabinofuranose products were analysed by thin-layer chromatography indicating alpha 1,5 hydrolytic activity (a). Rv3707c was incubated with mycobacterial arabinogalactan at the indicated pHs and the reaction products were similarly analysed. The enzyme is most active near PH 7 and generates fewer fragments from mycobacterial arabinogalactan indicating increased enzymatic specificity (b). Black arrows indicate enzymatically derived D-arabinofuranose oligomers.

FIG. 9 shows activity of additional endo-D-arabinanase enzymes. 1 μM of Gordonia phage GMA6 DUF4185 enzyme (G. phage), M. xanthus DUF4185 enzyme (MXAN_4606) and M. abscessus DUF 4185 enzyme MYCMA_14050 (M. abs Ga0) were incubated overnight at 37° C. with a) AG, b) LAM or c) P. aeruginosa pilin oligosaccharides. Products were analysed by thin-layer chromatography, stained with orcinol.

FIG. 10 shows Activity of DUF2961 enzymes against α-D-arabino configured substrates. DUF2961 enzymes activity against AG, LAM and α-1,5-arabino-oligosaccharides from P. aeruginosa pilins. Enzymes at 1 μM were incubated with substrates overnight at 37° C. a) D. gadei 02479 b) M. amalyticus DUF2961 and c) N. brasiliensis DUF2961

FIG. 11 shows enzyme activity against mycobacterial arabinogalactan. Activity of 02479 against D-arabino configured substrates. a) Rate of arabinose release from M. smegmatis by 100 nM 02479 over the specified time periods. Stopped assays were analysed by HPAEC-PAD and quantified using a D-arabinose standard curve and a fucose internal standard. b) Activity against pNP-α-D-arabinofuranoside in 20 mM HEPES pH 7.5, measured through a continuous assay by monitoring release of pNP at 405 nm. Reactions were initiated by the addition of 100 nM 02479.

FIG. 12 shows activity of 02470 on arabinan-containing substrates. a) TLC of 1 μM 02470 incubated overnight with AG, LAM and α-D-1,5-arabino-oligosaccharides in 20 mM HEPES pH 7.5. b) Rate of arabinose release from AG by 25 nM 02470 over the specified time periods. Stopped assays were analysed by HPAEC-PAD and quantified using a D-arabinose standard curve and a fucose internal standard.

FIG. 13 shows a cocktail of enzymes can completely hydrolyse M. smegmatis AG. 1 μM each of DG_02470, 79, 80 and 81, BACFIN_04787 and BACFIN_00810 were incubated overnight with 1 mg/ml AG in 20 mM HEPES pH 7.5. TLC analysis shows total hydrolysis to arabinose and galactose.

FIG. 14 shows qubit quantification of M. smegmatis genomic DNA from enzymatic lysis and bead-beating. Enzyme concentrations and incubation as listed in Table 1 and described above. Bead-beating in PBS was achieved using 100 μl of silica beads and 45 s of bead-beating.

FIG. 15 shows enzyme cocktail lysis incubation-time optimisation. Lysis, DNA purification and quantification were carried out as above using the indicated incubation times for a) M. smegmatis and b) M. bovis BCG. Bead-beating, the current standard procedure, was used as a control for M. smegmatis and a 2 hour incubation with only commercially available lipase and lysozyme for M. bovis BCG.

FIG. 16 shows results of multiplex PCR from M. smegmatis genomic DNA. 1 μl of genomic DNA from bead-beaten cells or cells incubated with enzyme lysis mix for 15, 30, 60 or 120 minutes was used as template for PCR reaction. Each time point was run in duplicate.

FIG. 17 shows different enzyme combinations. Yield of M. bovis BCG gDNA after two hours of lysis using conditions as described above, with enzymes as in Table 4, with a different enzyme excluded in each case.

FIG. 18 shows that galactan-degrading enzymes contribute to lysis. Corynebacterium glutamicum ΔaftA was incubated either with lysozyme or BACFIN_04787 at 5 UM and the change in optical density monitored after 18 h.

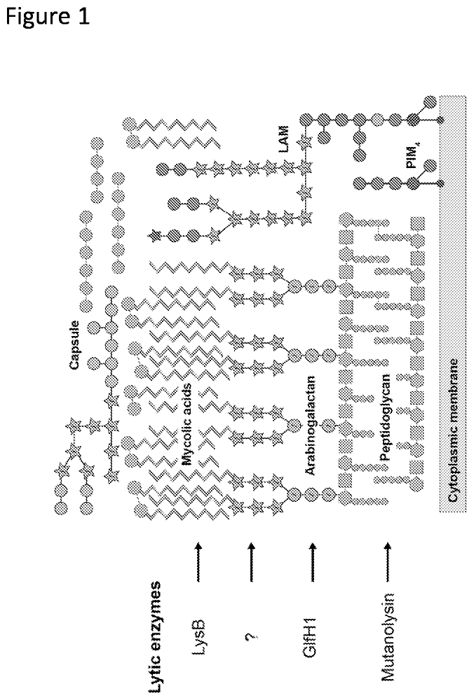

### DETAILED DESCRIPTION

The invention is based on the inventors' characterisation of enzymes with previously unknown function. Advantageously, these new enzymes can degrade mycobacterial arabinogalactan by cleaving linkages between two D-galactofuranoses and/or two D-arabinofuranoses. In the present disclosure, when reference is made to degrading arabinogalactan, it will be appreciated that this refers to cleaving linkages between two D-galactofuranoses and/or two D-arabinofuranoses. As explained in more detail hereinbelow, some of the newly characterised enzymes have the ability to cleave linkages between two D-galactofuranoses, while others have the ability to cleave linkages between two D-arabinofuranoses. Suitably, these D-galactofuranoses and/or D-arabinofuranoses may be part of the arabinogalactan component of a bacterial cell well. In particular bacteria of the Actinomycetota phylum, such as mycobacteria comprise arabinogalactan within their cell walls. By having the ability to degrade the arabinogalactan component of the bacterial cell wall, these enzymes may be particularly useful for lysing bacteria of the Actinomycetota phylum.

Cleaving the linkages between two D-galactofuranoses and/or two D-arabinofuranoses in order to lyse bacteria of the Actinomycetota phylum, is one exemplary utilisation of the enzymes described herein. Other uses of the enzymes described herein involve, for example, cleaving linkages between two D-galactofuranoses and/or two D-arabinofuranoses in order to produce D-galactofuranose polymers and/or D-arabinofuranose polymers from raw materials, such as arabinogalactans and/or lipoarabinomannan derived from bacteria.

### Uses of Enzymes

In one aspect, the present invention provides use of an enzyme for cleaving a linkage between two D-galactofuranoses in a polysaccharide, wherein the enzyme comprises an amino acid sequence having at least 70% sequence identity to a sequence provided in Table 1.

In another aspect, the present invention provides use of an enzyme for cleaving a linkage between two D-arabinofuranoses in a polysaccharide, wherein the enzyme comprises an amino acid sequence having at least 70% sequence identity to a sequence provided in Table 2.

TABLE 1
Enzymes for cleaving a linkage between two D-galactofuranoses

| | | Tested | Exemplary | Full length |
|---|---|---|---|---|
| | | enzyme | construct | protein |
| Enzyme type | Construct Name | SEQ ID NO: | SEQ ID NO: | SEQ ID NO: |
| exo-D- | BACFIN_04787 | 1 | 15 | 29 |
| galactofuranosidase | | | | |
| exo-D- | BACFIN_08811 | 2 | 16 | 30 |
| galactofuranosidase | | | | |
| endo-D- | BACFIN_08810 | 3 | 17 | 31 |
| galactofuranase | | | | |

TABLE 2
Enzymes for cleaving a linkage between two D-arabinofuranoses

| | | Tested | Exemplary | Full length |
|---|---|---|---|---|
| | | enzyme | construct | protein |
| Enzyme type | Construct Name | SEQ ID NO: | SEQ ID NO: | SEQ ID NO: |
| Endo-D- | MYCMA_14050 | 4 | 18 | 32 |
| arabinofuranase | | | | |
| Endo-D- | HMPREF9455_02480 | 5 | 19 | 33 |
| arabinofuranase | | | | |
| Endo-D- | HMPREF9455_02481 | 6 | 20 | 34 |
| arabinofuranase | | | | |
| Endo-D- | Rv3707c | 7 | 21 | 35 |
| arabinofuranase | | | | |
| Endo-D- | MXN_4606 | 8 | 22 | 36 |
| arabinofuranase | | | | |
| Endo-D- | GordoniaPhage | 9 | 23 | 37 |
| arabinofuranase | GMA6_4185 | | | |
| exo-D- | O3I_017420 | 10 | 24 | 38 |
| arabinofuranosidase | | | | |
| exo-D- | HMPREF9455_02479 | 11 | 25 | 39 |
| arabinofuranosidase | | | | |
| exo-D- | HMPREF9455_02467 | 12 | 26 | 40 |
| arabinofuranosidase | | | | |
| exo-D- | HMPREF9455_02470 | 13 | 27 | 41 |
| arabinofuranosidase | | | | |
| exo-D- | GII36_05205 | 14 | 28 | 42 |
| arabinofuranosidase | | | | |

As used herein, the term “enzyme” refers to a protein that catalyses a chemical reaction. In the context of the present disclosure the chemical reaction may be cleavage of a linkage between two D-galactofuranoses and/or cleavage of a linkage between two D-arabinofuranoses.

A linkage is cleaved when a bond between two D-galactofuranose and/or two D-arabinofuranose molecules is broken, thereby chemically separating the two molecules.

It will be appreciated that one or both of the two separated molecules (D-galactofuranose or D-arabinofuranose molecules) may part of a longer chain (i.e. a polymer) of D-galactofuranose or D-arabinofuranose. Such longer chains of D-galactofuranose or D-arabinofuranose may be found in the arabinogalactan component of a cell well from a bacterium of the Actinomycetota phylum (such as a mycobacterium). Suitably, a linkage between two D-galactofuranoses and/or two D-arabinofuranoses may be cleaved by hydrolysis (i.e. breakage of a bond by the addition of the elements of a water molecule).

Suitably, an enzyme for cleaving a linkage between two D-galactofuranoses may be an exo-D-galactofuranosidase and/or an endo-D-galactofuranase.

In the context of the present disclosure, an exo-D-galactofuranosidase is an enzyme that cleaves a linkage between two D-galactofuranoses so as to release an outermost D-galactofuranose molecule from a D-galactofuranose polymer. In other words, an exo-D-galactofuranosidase is an enzyme that cleaves off a monomer (one molecule) of D-galactofuranose at a time from a D-galactofuranose polymer. The D-galactofuranose polymer may be of any length. Merely by way of example the polymer may consist or comprise of 2, 3, 4, 5, 6, 7, 8, 9, 10 or more D-galactofuranose molecules. For example, the polymer may consist or comprise of 15, 20, 25, 30, 35, 40, 45, 50, 55, 60 or more D-galactofuranose molecules.

Suitably, the exo-D-galactofuranosidase is an enzyme capable of cleaving β1-5 and/or of β1-6 linkages between two D-galactofuranoses.

Suitably, the exo-D-galactofuranosidase may comprise an amino acid sequence as shown in SEQ ID NO: 1 or SEQ ID NO: 2; or an amino acid sequence which has at least 70% (for example at least 75%, at least 80%, at least 85%, at least 90%, at least 95%, at least 96%, at least 97%, at least 98%, at least 99%, or more) sequence identity thereto. More suitably, the exo-D-galactofuranosidase may comprise an amino acid sequence as shown in SEQ ID NO: 1 or SEQ ID NO: 2. An example of an exo-D-galactofuranosidase that comprises SEQ ID NO: 1 is shown in SEQ ID NO: 29. An example of an exo-D-galactofuranosidase that comprises SEQ ID NO: 2 is shown in SEQ ID NO: 30.

Suitably, the exo-D-galactofuranosidase may consist of an amino acid sequence as shown in SEQ ID NO: 1 or SEQ ID NO: 2. Alternatively, the exo-D-galactofuranosidase may consist of an amino acid sequence as shown in SEQ ID NO: 29 or SEQ ID NO: 30.

In the context of the present disclosure, an endo-D-galactofuranase is an enzyme that cleaves the linkage between two D-galactofuranoses anywhere within a polymer of D-galactofuranose so as to break a polymer of D-galactofuranose molecules into two smaller (shorter length) polymers. Such smaller polymers may consist or comprise of two or more D-galactofuranose molecules.

Suitably, the endo-D-galactofuranase is an enzyme capable of cleaving β1-5 linkages between two D-galactofuranoses.

Suitably, the endo-D-galactofuranase may comprise an amino acid sequence as shown in SEQ ID NO: 3 or an amino acid sequence which has at least 70% (for example at least 75%, at least 80%, at least 85%, at least 90%, at least 95%, at least 96%, at least 97%, at least 98%, at least 99%, or more) sequence identity thereto.

More suitably, the endo-D-galactofuranase may comprise an amino acid sequence as shown in SEQ ID NO: 3. An example of an endo-D-galactofuranase that comprises an amino acid sequence as shown in SEQ ID NO: 3 is shown in SEQ ID NO: 31.

Suitably, the endo-D-galactofuranase may consist of an amino acid sequence as shown in SEQ ID NO: 3. Alternatively, the endo-D-galactofuranase may consist of an amino acid sequence as shown in SEQ ID NO: 31.

Suitably, an enzyme for cleaving a linkage between two D-arabinofuranoses may be an endo-D-arabinofuranase and/or exo-D-arabinofuranosidase.

In the context of the present disclosure, an endo-D-arabinofuranase is an enzyme that cleaves the linkage between two D-arabinofuranoses anywhere within a D-arabinofuranose polymer so as to break the polymer into two smaller (shorter length) polymers. Such smaller polymers may consist or comprise of two or more D-arabinofuranose molecules.

Suitably, the endo-D-arabinofuranase is an enzyme capable of cleaving β1-5 and/or β1-3 linkages between two D-arabinofuranoses.

Suitably, the endo-D-arabinofuranase may comprise an amino acid sequence as shown in SEQ ID NO: 4, SEQ ID NO: 5, SEQ ID NO: 6, SEQ ID NO: 7, SEQ ID NO: 8, or SEQ ID NO: 9; or an amino acid sequence which has at least 70% (for example at least 75%, at least 80%, at least 85%, at least 90%, at least 95%, at least 96%, at least 97%, at least 98%, at least 99%, or more) sequence identity thereto. Suitably, the endo-D-arabinofuranase may comprise an amino acid sequence as shown in SEQ ID NO: 4, SEQ ID NO: 5, or SEQ ID NO: 6; or an amino acid sequence which has at least 70% (for example at least 75%, at least 80%, at least 85%, at least 90%, at least 95%, at least 96%, at least 97%, at least 98%, at least 99%, or more) sequence identity thereto.

More suitably, the endo-D-arabinofuranase may comprise an amino acid sequence as shown in SEQ ID NO: 4, SEQ ID NO: 5, SEQ ID NO: 6, SEQ ID NO: 7, SEQ ID NO: 8, or SEQ ID NO: 9. Suitably, the endo-D-arabinofuranase may comprise an amino acid sequence as shown in SEQ ID NO: 4, SEQ ID NO: 5, or SEQ ID NO: 6.

An example of a endo-D-arabinofuranase that comprises SEQ ID NO: 4 is shown in SEQ ID NO: 32. An example of a endo-D-arabinofuranase that comprises SEQ ID NO: 5 is shown in SEQ ID NO: 33. An example of a endo-D-arabinofuranase that comprises SEQ ID NO: 6 is shown in SEQ ID NO: 34. An example of a endo-D-arabinofuranase that comprises SEQ ID NO: 7 is shown in SEQ ID NO: 35. An example of a endo-D-arabinofuranase that comprises SEQ ID NO: 8 is shown in SEQ ID NO: 36. An example of a endo-D-arabinofuranase that comprises SEQ ID NO: 9 is shown in SEQ ID NO: 37.

Suitably, the endo-D-arabinofuranase may consist of an amino acid sequence as shown in SEQ ID NO: 4, SEQ ID NO: 5, SEQ ID NO: 6, SEQ ID NO: 7, SEQ ID NO: 8, or SEQ ID NO: 9. Suitably, the endo-D-arabinofuranase may consist of an amino acid sequence as shown in SEQ ID NO: 4, SEQ ID NO: 5 or SEQ ID NO: 6.

Alternatively, the endo-D-arabinofuranase may consist of an amino acid sequence as shown in SEQ ID NO: 32, SEQ ID NO: 33, SEQ ID NO: 34, SEQ ID NO: 35, SEQ ID NO: 36, or SEQ ID NO: 37. Suitably, the endo-D-arabinofuranase may consist of an amino acid sequence as shown in SEQ ID NO: 32, SEQ ID NO: 33, or SEQ ID NO: 34.

In the context of the present disclosure, an exo-D-arabinofuranosidase is an enzyme that cleaves a linkage between two D-arabinofuranoses so as to release an outermost D-arabinofuranose molecule from a D-arabinofuranose polymer. In other words, an exo-D-arabinofuranosidase is an enzyme that cleaves off a monomer (one molecule) of D-arabinofuranose at a time from a D-arabinofuranose polymer. The D-arabinofuranose polymer may be of any length. Merely by way of example the polymer may consist or comprise of 2, 3, 4, 5, 6, 7, 8, 9, 10 or more D-arabinofuranose molecules. For example, the polymer may consist or comprise of 15, 20, 25, 30, 35, 40, 45, 50, 55, 60 or more D-arabinofuranose molecules.

Suitably, the exo-D-arabinofuranosidase is an enzyme capable of cleaving α1-5 and/or of α1-3 linkages between two D-arabinofuranose. Suitably the exo-D-arabinofuranosidase comprising an amino acid sequence as shown in SEQ ID NO: 10, SEQ ID NO: 11, SEQ ID NO: 12, SEQ ID NO: 13, or SEQ ID NO: 14, may be capable of cleaving α1-5 linkages and/or of α1-3 linkages between two D-arabinofuranose. Suitably, exo-D-arabinofuranosidase comprising an amino acid sequence as shown in SEQ ID NO: 10, SEQ ID NO: 11, SEQ ID NO: 12, or SEQ ID NO: 14, may be capable of cleaving α1-5 linkages between two D-arabinofuranose.

Suitably, the exo-D-arabinofuranosidase may comprise an amino acid sequence as shown in SEQ ID NO: 10, SEQ ID NO: 11, SEQ ID NO: 12, SEQ ID NO: 13, or SEQ ID NO: 14; or an amino acid sequence which has at least 70% (for example at least 75%, at least 80%, at least 85%, at least 90%, at least 95%, at least 96%, at least 97%, at least 98%, at least 99%, or more) sequence identity thereto.

Suitably, the exo-D-arabinofuranosidase may comprise an amino acid sequence as shown in SEQ ID NO: 10, SEQ ID NO: 11, SEQ ID NO: 12, or SEQ ID NO: 13; or an amino acid sequence which has at least 70% (for example at least 75%, at least 80%, at least 85%, at least 90%, at least 95%, at least 96%, at least 97%, at least 98%, at least 99%, or more) sequence identity thereto.

More suitably, the exo-D-arabinofuranosidase may comprise an amino acid sequence as shown in SEQ ID NO: 10, SEQ ID NO: 11, SEQ ID NO: 12, SEQ ID NO: 13, or SEQ ID NO: 14.

Suitably, the exo-D-arabinofuranosidase may comprise an amino acid sequence as shown in SEQ ID NO: 10, SEQ ID NO: 11, SEQ ID NO: 12, or SEQ ID NO: 13.

An example of an exo-D-arabinofuranosidase that comprises SEQ ID NO: 10 is shown in SEQ ID NO: 38. An example of an exo-D-arabinofuranosidase that comprises SEQ ID NO: 11 is shown in SEQ ID NO: 39. An example of an exo-D-arabinofuranosidase that comprises SEQ ID NO: 12 is shown in SEQ ID NO: 40. An example of an exo-D-arabinofuranosidase that comprises SEQ ID NO: 13 is shown in SEQ ID NO: 41. An example of an exo-D-arabinofuranosidase that comprises SEQ ID NO: 14 is shown in SEQ ID NO: 42.

Suitably, the exo-D-arabinofuranosidase may consist of an amino acid sequence as shown in SEQ ID NO: 10, SEQ ID NO: 11, SEQ ID NO: 12, SEQ ID NO: 13, or SEQ ID NO: 14. Suitably, the exo-D-arabinofuranosidase may consist of an amino acid sequence as shown in SEQ ID NO: 10, SEQ ID NO: 11, SEQ ID NO: 12, or SEQ ID NO: 13.

Alternatively, the exo-D-arabinofuranosidase may consist of an amino acid sequence as shown in SEQ ID NO: 38, SEQ ID NO: 39, SEQ ID NO: 40, or SEQ ID NO: 41.

As touched upon elsewhere in the present disclosure, the enzymes disclosed herein are capable of breaking linkages between two D-galactofuranoses and/or two D-arabinofuranoses. Such linkages may be naturally found in polysaccharides, such as bacterial cell wall polysaccharides. The term “polysaccharide” as used herein refers to a polymer composed of two or more monosaccharides linked to one another. Suitably, the polysaccharide may be selected from the group consisting of arabinogalactan, lipoarabinomannan (LAM) and pilin oligosaccharide.

As used herein the term “arabinogalactan” refers to a polysaccharide consisting or comprising of an arabinose polymer and a galactose polymer. The arabinose and galactose components of the polymers are in furanose form. Such arabinogalactan is known to be a major component of bacterial cell wells, in particular cell walls of bacteria of the Actinomycetota phylum (such as mycobacteria). In bacterial cell walls, typically arabinogalactan is anchored to the peptidoglycan via a conserved linker unit and the galactan portion of arabinogalactan is linear, consisting of approximately 30 residues of galactan with alternating β1-5 and β1-6 glycosidic linkages. The arabinan portion typically consists of approximately 30 residues of arabinan which are attached at three branch points within the galactan chain (believed to be at residues 8, 10 and 12). The arabinan portion of the polymer is a complex branched structure, usually capped with mycolic acids; the arabinan glycosidic linkages are α1-3, α1-5, and β1-2. The enzymes described herein are useful in the context of bacterial lysis due to their ability to degrade the arabinogalactan cell well component by breaking linkages which form the arabinose polymer and the galactose polymer of arabinogalactan.

It will be appreciated that because arabinogalactan comprises polymers of both arabinan and galactan, arabinogalactan may be degraded by enzymes that cleave a linkage between two D-galactofuranoses and/or two D-arabinofuranoses. Thus the enzymes newly characterised by the inventors, i.e. the exo-D-galactofuranosidases, the endo-D-galactofuranases, the endo-D-arabinofuranases, and/or the exo-D-arabinofuranosidases described herein, may be referred to as arabinogalactan degrading enzymes. Furthermore, more broadly speaking, each of these enzymes is an example of a bacterial cell wall degrading enzyme.

In some embodiments the polysaccharide may be lipoarabinomannan (LAM) or pilin oligosaccharide. LAM is a polysaccharide which comprises D-arabinofuranose polymers and D-mannopyranose polymers, and is attached to a lipid anchor. Typically, the arabinan polymer of LAM consists of a linear backbone of α1-5 linked D-arabinofuranoses with branched hexa-arabinofuranosides and linear tetra-arabinofuranosides. Pilin oligosaccharide is an oligosaccharide which comprises of α1,5-arabinofuranose residues removed from the pili of Pseudomonas aeruginosa PA7. Accordingly the endo-D-arabinofuranase and/or exo-D-arabinofuranosidase enzymes as described herein, due to their ability to degrade a linkage between two D-arabinofuranoses, may degrade lipoarabinomannan (LAM) and/or pilin oligosaccharide.

For avoidance of doubt, in the present disclosure the terms “D-galactofuranose”, “D-galactofuranose molecule”, “D-galactofuranose monomer, “galactan” and “galactan molecule” refer to the same compound having a structure as shown in Formula I of Table 3. By the same token, in the present disclosure the terms “D-arabinofuranose”, “D-arabinofuranose molecule”, “D-arabinofuranose monomer” “arabinan” and “arabinan molecule” refer to the same compound having a structure as shown in Formula II of Table 3. Furthermore, herein the terms “chain” and “polymer” may be used interchangeably.

TABLE 3
Chemical structure depiction of D-galactofuranose and D-arabinofuranose

| D-Galactofuranose | D-Arabinofuranose |
|---|---|
| 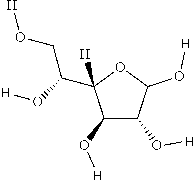 [SMILES: OC[C@@H](O)[C@@H]1OC(O)[C@H](O)[C@H]1O] | 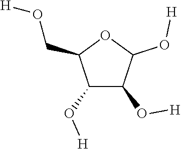 Formula II |
| Formula 1 | |

The term “sequence identity” as used herein refers to determination of the identity between a reference amino acid sequence and a query sequence wherein the sequences are aligned so that the highest order match is obtained, and which can be calculated using published techniques or methods codified in computer programs such as, for example, BLASTP, BLASTN, FASTA (Altschul 1990, J Mol Biol 215:403). The percent identity values may be calculated over the entire amino acid sequence or over fragment of the amino acid sequence. It will be appreciated that the reference sequence may be any one of the amino acid sequences shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14. An amino acid sequence that shares less than 100% sequence identity with the reference sequence may be referred to as a variant. It will be appreciated that in order to cleave a linkage between two D-galactofuranoses and/or two D-arabinofuranose, the variant must be a functional variant.

Suitably, the variant may contain only a conservative substitution of one or more amino acids of the reference sequence (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14), or a substitution, a deletion or an insertion of non-critical amino acids in non-critical regions of the sequence. As used herein, the term “conservative substitution” means substitution between amino acids having the same property (basic, acidic, or neutral) or the same polarity (hydrophilic or hydrophobic) or between aromatic amino acids, or between aliphatic amino acids, for example, substitution from basic to basic, acidic to acidic, or polar to polar. The conservative substitution is performed, for example, within each of the groups of basic amino acids (Arg, Lys, His), acidic amino acids (Glu, Asp), neutral non-polar amino acids (Gly, Ala, Val, Leu, Ile, Met), aliphatic amino acids (Ala, Val, Leu, Ile, Met), polar amino acids (Gln, Asn, Ser, Thr), and aromatic amino acids (Phe, Trp, Tyr). Methods for identifying conservative substitutions of amino acids are well known in the art (see, for example, Brummell et al, Biochem. 32:1180-1187 (1993); Kobayashi et al., Protein Eng. 12 (10): 879-884 (1999) and Burks et al. Proc. Natl Acad. Set USA 94:412-417 (1997)).

The phrase “at least 70% sequence identity” as used herein means at least about 70%, at least 75%, at least 80%, at least 85%, at least 90%, at least 91%, at least 92%, at least 93%, at least 94%, at least 95%, at least 96%, at least 97%, at least 98%, at least 99% or 100% identity with SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14, or fragment thereof. The fragment over which the amino acid sequence identity may be calculated may be up to 50 amino acid (aa) residues, up to 100aa, up to 150aa, up to 250aa, 300aa, 350aa, 400aa, 450aa, 500aa, 550aa, 600aa, or more residues, up to the full-length of the reference amino acid sequence. It will be appreciated that in order to cleave a linkage between two D-galactofuranoses and/or two D-arabinofuranose, the fragment must be a functional fragment.

By term “functional” in the context of a variant or fragment, it is meant that the variant or fragment retains at least partial biological function that is similar to or substantially the same as compared to the reference amino acid sequence upon which said variant or fragment is based. In the context of the present invention it will be appreciated that the biological function for SEQ ID NO: 1, SEQ ID NO: 2, and/or SEQ ID NO: 3 is the ability to cleave a linkage between two D-galactofuranose molecules. The biological function for SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and/or SEQ ID NO: 14 is the ability to cleave a linkage between two D-arabinofuranose molecules.

Several suitable methods may be used to determine whether a fragment or variant retains the ability to cleave a linkage between two D-galactofuranose molecules and/or two D-arabinofuranose molecules. Details of one such method are provided in the Examples section of the present disclosure. Merely by way of example, the method may involve producing the protein recombinantly for example by introducing the gene on an inducible expression plasmid in a suitable strain of Escherichia coli and inducing its expression with isopropyl-β-D-thiogalactopyranoside (IPTG). The protein can be purified using standard techniques. Purified protein can then be incubated with purified arabinogalactan, LAM or pilin oligosaccharide and analysed by, for example, thin-layer chromatography to determine its ability to cut between two D-galactofuranose molecules and/or two D-arabinofuranose molecules. Suitable strains of Escherichia coli may be those that have to have a genetic background to enable the plasmid containing the gene to express the protein. Merely by way of example a suitable strain is E. coli BL21 Tuner (DE3). Said strain has the required genetics to use IPTG based induction based on the lac operon. IPTG based induction is a common feature of pET vectors which are plasmids used for the expression of genes in E. coli using the lac operon. Suitably, the desired gene (for example a gene encoding an enzyme of the invention, or an enzyme suspected of having the ability to cleave a linkage between two D-galactofuranose molecules or between two D-arabinofuranose molecules) may be cloned into pET28a with an in-frame C-terminal hexa-histidine tag. This plasmid may be then transformed into E. coli BL21 Tuner (DE3). Cultures of this strain may be grown with shaking at 37° C. to an optical density of 0.6 at which point protein production may be induced through the addition of IPTG (at a concentration of for example around 0.5 mM) after which culture temperature may be reduced to 18° C. overnight. The cells may be subsequently collected by centrifugation and lysed and the protein purified using standard immobilised metal affinity chromatography techniques.

As mentioned, linkages between two D-galactofuranoses and/or two D-arabinofuranoses may be found in bacterial cell wall polysaccharides of bacteria of the Actinomycetota phylum.

Suitably, the bacterium may be of the Mycobacteriales order.

Suitably, the bacterium may be of the family selected from the group consisting of: Segniliparaceae, Mycobacteriaceae, Nocardiaceae, Tsukamurellaceae, Gordoniaceae, Lawsonellaceae, Corynebacteriaceae and Dietziaceae.

Suitably, the bacterium may be selected from the group consisting of M. tuberculosis, M. bovis, M. africanum, M. canetti, M. microti. M. smegmatis, M. fortuitum, M. marinum, M. ulcerans, M. paratuberculosis, M. celatum, M. avium, M. leprae, M. lepraemurium, M. intracellulare M. scrofulaceum, M. xenopi, M. genavense, M. kansasii, M. simiae, M. szulgai, M. haemophilum, M. asiaticum, M. malmoense, M. vaccae, M. caprae, M. pinnipe dii or M. shimoidei. More suitably the bacterium may be M. tuberculosis.

Suitably, the bacterium of the Tsukamurellaceae family may be Tsukamurella paurometabola.

Suitably, the bacterium of the Nocardiaceae family may be selected from Nocardia farcinia and Nocardia brasiliensis.

### Compositions of the Invention

In a further aspect, the present invention provides a composition for degrading a Actinomycetota bacterial cell wall polysaccharide.

Suitably, the composition may comprise at least two bacterial cell wall degrading enzymes.

The term “bacterial cell wall degrading enzyme” as used herein refers to an enzyme that is capable of cleaving linkages in a bacterial cell wall. Suitably the linkages may be within or between cell wall components. Linkages between cell wall components are those that link two different types of cell wall component (for example link mycolic acid with arabinogalactan). Linkages within cell wall components are those that link two molecules of the same cell wall component (for example link two D-galactofuranose molecules of the arabinogalactan component).

Suitably, the bacterial cell wall component may be selected from the group consisting of arabinogalactan, peptidoglycan, mycolic acid, lipoarabinomannan (LAM) and pilin oligosaccharide. Each of these components are typically found in cell walls of bacteria from the Actinomycetota phylum. By cleaving linkages in one or more of these components, and/or between these components, it can be said that the enzyme degrades said component, and thereby degrade the bacterial cell wall. As mentioned elsewhere in the present disclosure, an enzyme that cleaves linkages in arabinogalactan can be said to degrade it (i.e. is an arabinogalactan degrading enzyme). Such an enzyme may be an exo-D-galactofuranosidase, endo-D-galactofuranase, exo-D-arabinofuranase and/or endo-D-arabinofuranosidase.

Suitably an enzyme that degrades arabinogalactan may have an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or an amino acid sequence which has at least 70% sequence identity thereto. For avoidance of doubt, a bacterial cell wall degrading enzyme that degrades peptidoglycan may be referred to herein as “a peptidoglycan degrading enzyme”, a bacterial cell wall degrading enzyme that degrades mycolic acid may be referred to herein as “a mycolic acid degrading enzyme”, a bacterial cell wall degrading enzyme that degrades LAM be referred to herein as “a LAM degrading enzyme”, and a bacterial cell wall degrading enzyme that degrades pilin oligosaccharide may be referred to herein as “a pilin oligosaccharide degrading enzyme”.

The term “peptidoglycan” as used herein refers to a glycopeptide polymer that is a component of bacterial cell walls, including Gram-positive and Gram-negative bacteria. Peptidoglycan is generally characterised as containing N-acetyl- or N-glycolylmuramic acid and D-amino acids. As mentioned, a “peptidoglycan degrading enzyme” is an enzyme that cleaves linkages in the peptidoglycan polymer. Suitably, the peptidoglycan degrading enzymes may have a hydrolase activity. Many peptidoglycan degrading enzymes are known in the art. Merely by way of example, the peptidoglycan degrading enzyme may be selected from the group consisting of a lysozyme (for example hen-egg white lysozyme), mutanolysin, muramidase, glucosaminidase, transglycosylase, amidase, endopeptidase and/or endolysin.

Lysine A (LysA) enzymes are examples of enzymes having muramidase and/or amidase activity. The main classes of muramidase enzymes of commercial relevance include hen egg white lysozymes (HEWL) and mutanolysin which belong to GH22 and GH25 families respectively. While both enzyme families may cleave the glycan backbone of unmodified peptidoglycan, GH22-type lysozymes have a more closed active site and may be therefore inhibited by modifications to the backbone whereas GH25 enzymes are not. This is of particular importance in the context of mycobacterial lysis where the peptidoglycan is known to be heavily modified. Nonetheless, GH22-type lysozymes are expected to have some, albeit weaker, activity against the mycobacterial peptidoglycan.

The term “mycolic acid” as used herein refers to an α-alkyl-β-hydroxyl fatty acid with a total carbon number of about 22 to 90. Depending on bacterium, the total carbon number in the fatty acid may vary. For example, Mycobacterium species may include a short saturated alpha, C20-25, and a longer meromycolate chain, the beta-hydroxy branch C60, comprising double bonds, cyclopropane rings and oxygenated groups. As mentioned, a “mycolic acid degrading enzyme” is an enzyme that cleaves linkages in mycolic acid and/or cleaves linkages between mycolic acid and arabinogalactan. Suitably, the mycolic acid degrading enzyme may be Lysin B (LysB) or Candida rugosa lipase. LysB enzymes cleave the ester linkage between the mycolic acids and arabinogalactan, aiding in permeabilization of the cell wall. These enzymes belong to a large structural superfamily of a/β hydrolase enzymes which act on a wide diversity of substrates.

The term “lipoarabinomannan” and “pilin oligosaccharide” are defined elsewhere in the present specification.

The term “lipoarabinomannan degrading enzyme” refers to an enzyme that cleaves linkages within and/or between D-arabinofuranose and/or D-mannopyranose polymers, and/or cleaves linkages between D-mannopyranose polymers and the lipid anchor. The exo-D-arabinofuranase and/or endo-D-arabinofuranosidase enzymes as described herein are examples of lipoarabinomannan degrading enzymes. The term “pilin oligosaccharide degrading enzyme” refers to an enzyme that cleaves a linkage between two α1,5-arabino-monosaccharides.

Suitably, the first of the at least two cell wall degrading enzymes in the composition is an enzyme that comprises an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14; or an amino acid sequence which has at least 70% sequence identity thereto.

Suitability, the first of the at least two cell wall degrading enzymes in the composition is an enzyme that comprises an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 10, SEQ ID NO: 11, SEQ ID NO: 12, or SEQ ID NO: 13; or an amino acid sequence which has at least 70% sequence identity thereto.

More suitably, the first of the at least two cell wall degrading enzymes may be an enzyme that comprises an amino acid sequence as shown in SEQ ID NO: 1, SEQ ID NO: 2, or SEQ ID NO: 3. Most suitably, the first of the at least two cell wall degrading enzymes may be an enzyme that comprises an amino acid sequence as shown in SEQ ID NO: 1 or SEQ ID NO: 2.

Suitably, the second of the at least two cell wall degrading enzymes in the composition is selected from the group consisting of arabinogalactan degrading enzyme, peptidoglycan degrading enzyme, mycolic acid degrading enzyme, lipoarabinomannan degrading enzyme and pilin oligosaccharide degrading enzyme.

Suitably, the composition comprises at least one arabinogalactan degrading enzyme comprising an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or an amino acid sequence which has at least 70% sequence identity thereto, at least one peptidoglycan degrading enzyme, and/or least one mycolic acid degrading enzyme. The peptidoglycan degrading enzyme may selected from the group consisting of a lysozyme (for example hen-egg white lysozyme), mutanolysin, muramidase, glucosaminidase, transglycosylase, amidase, endopeptidase and/or endolysin. The mycolic acid may be Lysin B or lipase (for example Candida rugosa lipase).

More suitably the composition comprises at least one arabinogalactan degrading enzyme comprising an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 10, SEQ ID NO: 11, SEQ ID NO: 12, or SEQ ID NO: 13; or an amino acid sequence which has at least 70% sequence identity thereto, at least one peptidoglycan degrading enzyme, and/or least one mycolic acid degrading enzyme. The peptidoglycan degrading enzyme may selected from the group consisting of a lysozyme (for example hen-egg white lysozyme), mutanolysin, muramidase, glucosaminidase, transglycosylase, amidase, endopeptidase and/or endolysin. The mycolic acid may be Lysin B or lipase (for example Candida rugosa lipase).

Suitably, the second of the at least two cell wall degrading enzymes is an arabinogalactan degrading enzyme. Suitably, the arabinogalactan degrading enzyme may be an enzyme that comprises an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or an amino acid sequence which has at least 70% sequence identity thereto.

More suitably, the second of the at least two enzymes is an enzyme that comprises an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 10, SEQ ID NO: 11, SEQ ID NO: 12, or SEQ ID NO: 13; or an amino acid sequence which has at least 70% sequence identity thereto.

In one example, the composition may comprise:
a) at least one enzyme comprising an amino acid sequence as shown in SEQ ID NO: 1, SEQ ID NO: 2, or SEQ ID NO: 3; or an amino acid sequence which has at least 70% sequence identity thereto; and
b) at least one enzyme comprising an amino acid sequence as shown in SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or an amino acid sequence which has at least 70% sequence identity thereto. In other words, the composition may comprise at least one enzyme capable of cleaving a linkage between two D-galactofuranoses, and at least one enzyme capable of cleaving a linkage between two D-arabinofuranoses.

In one example, the composition may comprise:
a) at least one enzyme comprising an amino acid sequence as shown in SEQ ID NO: 1, SEQ ID NO: 2, or SEQ ID NO: 3; or an amino acid sequence which has at least 70% sequence identity thereto; and
b) at least one enzyme comprising an amino acid sequence as shown in SEQ ID NO: 4, SEQ ID NO: 5, SEQ ID NO: 6, SEQ ID NO: 10, SEQ ID NO: 11, SEQ ID NO: 12, or SEQ ID NO: 13; or an amino acid sequence which has at least 70% sequence identity thereto. In other words, the composition may comprise at least one enzyme capable of cleaving a linkage between two D-galactofuranoses, and at least one enzyme capable of cleaving a linkage between two D-arabinofuranoses.

In one example, the composition may comprise an exo-D-galactofuranosidase comprising an amino acid sequence as shown in SEQ ID NO:1 or SEQ ID NO:2; or an amino acid sequence which has at least 70% sequence identity thereto.

In one example, the composition may comprise an endo-D-galactofuranase comprising an amino acid sequence as shown in SEQ ID NO:3; or an amino acid sequence which has at least 70% sequence identity thereto.

In one example, the composition may comprise an endo-D-arabinofuranase comprising an amino acid sequence as shown in SEQ ID NO:4, SEQ ID NO:5 or SEQ ID NO:6; or an amino acid sequence which has at least 70% sequence identity thereto.

In one example, the composition may comprise an exo-D-arabinofuranosidase comprising an amino acid sequence as shown in SEQ ID NO: 10, SEQ ID NO: 11, SEQ ID NO: 12, or SEQ ID NO: 13; or an amino acid sequence which has at least 70% sequence identity thereto.

In one example, the composition may comprise:
(i) an exo-D-galactofuranosidase comprising an amino acid sequence as shown in SEQ ID NO: 1 or SEQ ID NO:2; or an amino acid sequence which has at least 70% sequence identity thereto;
(ii) an endo-D-galactofuranase comprising an amino acid sequence as shown in SEQ ID NO: 3; or an amino acid sequence which has at least 70% sequence identity thereto; and
(iii) an endo-D-arabinofuranase comprising an amino acid sequence as shown in SEQ ID NO: 4, SEQ ID NO:5 or SEQ ID NO:6; or an amino acid sequence which has at least 70% sequence identity thereto. Optionally, the composition may further comprise an exo-D-arabinofuranosidase comprising an amino acid sequence as shown in SEQ ID NO: 10, SEQ ID NO: 11, SEQ ID NO: 12, or SEQ ID NO: 13; or an amino acid sequence which has at least 70% sequence identity thereto.

In one example, the composition may comprise an:
(i) an exo-D-galactofuranosidase comprising an amino acid sequence as shown in SEQ ID NO: 1 and/or SEQ ID NO: 2; or an amino acid sequence which has at least 70% sequence identity thereto;
(ii) an endo-D-galactofuranase comprising an amino acid sequence as shown in SEQ ID NO: 3; or an amino acid sequence which has at least 70% sequence identity thereto;
(iii) an endo-D-arabinofuranase comprising an amino acid sequence as shown in SEQ ID NO: 5 and/or SEQ ID NO: 6; or an amino acid sequence which has at least 70% sequence identity thereto; and
(iv) an exo-D-arabinofuranosidase comprising an amino acid sequence as shown in SEQ ID NO: 11 and/or SEQ ID NO: 13; or an amino acid sequence which has at least 70% sequence identity thereto.

In one example, the composition may comprise:
(i) an exo-D-galactofuranosidase comprising an amino acid sequence as shown in SEQ ID NO: 1 and/or SEQ ID NO: 2;
(ii) an endo-D-galactofuranase comprising an amino acid sequence as shown in SEQ ID NO: 3;
(iii) an endo-D-arabinofuranase comprising an amino acid sequence as shown in SEQ ID NO: 5 and/or SEQ ID NO: 6; and
(iv) an exo-D-arabinofuranosidase comprising an amino acid sequence as shown in SEQ ID NO: 11 and/or SEQ ID NO: 13.

In one example, the composition may comprise an:
(i) an exo-D-galactofuranosidase comprising an amino acid sequence as shown in SEQ ID NO: 1;
(ii) an endo-D-galactofuranase comprising an amino acid sequence as shown in SEQ ID NO: 3;
(iii) an endo-D-arabinofuranase comprising an amino acid sequence as shown in SEQ ID NO: 5 and SEQ ID NO: 6; and
(iv) an exo-D-arabinofuranosidase comprising an amino acid sequence as shown in SEQ ID NO: 11 and SEQ ID NO: 13.

In one example, the composition may comprise an:
(i) an exo-D-galactofuranosidase comprising an amino acid sequence as shown in SEQ ID NO: 1;
(ii) an endo-D-galactofuranase comprising an amino acid sequence as shown in SEQ ID NO: 3;
(iii) an endo-D-arabinofuranase comprising an amino acid sequence as shown in SEQ ID NO: 6; and
(iv) an exo-D-arabinofuranosidase comprising an amino acid sequence as shown in SEQ ID NO: 11 and SEQ ID NO: 13.

Each one of the exemplary compositions outlined herein above, may further comprise a peptidoglycan degrading enzyme, a mycolic acid degrading enzyme, lipoarabinomannan degrading enzyme and/or a pilin oligosaccharide degrading enzyme. Suitably, each one of the exemplary compositions outlined herein above, may further comprise a peptidoglycan degrading enzyme, and/or a mycolic acid degrading enzyme.

Suitably, each one of the exemplary compositions outlined herein above, may further comprise a peptidoglycan degrading enzyme and a mycolic acid degrading enzyme.

Accordingly, merely by way of example, the composition may comprise:
(i) an exo-D-galactofuranosidase comprising an amino acid sequence as shown in SEQ ID NO: 1;
(ii) an endo-D-galactofuranase comprising an amino acid sequence as shown in SEQ ID NO: 3;
(iii) an endo-D-arabinofuranase comprising an amino acid sequence as shown in SEQ ID NO: 5 and SEQ ID NO: 6;
(iv) an exo-D-arabinofuranosidase comprising an amino acid sequence as shown in SEQ ID NO: 11 and SEQ ID NO: 13;
(v) a peptidoglycan degrading enzyme (for example lysozyme, such as egg-hen lysozyme); and
(vi) a mycolic acid degrading enzyme (for example lipase, such as Candida rugosa lipase).

Suitably, the composition may comprise the enzymes as shown in Table 4.

Suitably the composition may comprise at least four of the five arabinogalactan degrading enzymes shown in Table 4. Optionally, the composition may further comprise a peptidoglycan degrading enzyme (for example lysozyme, such as egg-hen lysozyme); and/or a mycolic acid degrading enzyme (for example lipase, such as Candida rugosa lipase).

Accordingly, suitably, the composition may comprise an enzyme having an amino acid sequence as shown in SEQ ID NO: 13, an enzyme having an amino acid sequence as shown in SEQ ID NO: 6, an enzyme having an amino acid sequence as shown in SEQ ID NO: 3, and an enzyme having an amino acid sequence as shown in SEQ ID NO: 1, and optionally a peptidoglycan degrading enzyme (for example lysozyme, such as egg-hen lysozyme); and/or a mycolic acid degrading enzyme (for example lipase, such as Candida rugosa lipase).

Suitably, the composition may comprise an enzyme having an amino acid sequence as shown in SEQ ID NO: 11, an enzyme having an amino acid sequence as shown in SEQ ID NO: 6, an enzyme having an amino acid sequence as shown in SEQ ID NO: 3, and an enzyme having an amino acid sequence as shown in SEQ ID NO: 1, and optionally a peptidoglycan degrading enzyme (for example lysozyme, such as egg-hen lysozyme); and/or a mycolic acid degrading enzyme (for example lipase, such as Candida rugosa lipase).

Suitably, the composition may comprise an enzyme having an amino acid sequence as shown in an enzyme having an amino acid sequence as shown in SEQ ID NO: 13, an enzyme having an amino acid sequence as shown in SEQ ID NO: 11, an enzyme having an amino acid sequence as shown in SEQ ID NO: 3, and an enzyme having an amino acid sequence as shown in SEQ ID NO: 1, and optionally a peptidoglycan degrading enzyme (for example lysozyme, such as egg-hen lysozyme); and/or a mycolic acid degrading enzyme (for example lipase, such as Candida rugosa lipase).

Suitably, the composition may comprise an enzyme having an amino acid sequence as shown in SEQ ID NO: 13, an enzyme having an amino acid sequence as shown in SEQ ID NO: 11, an enzyme having an amino acid sequence as shown in SEQ ID NO: 6, and an enzyme having an amino acid sequence as shown in SEQ ID NO: 1, and optionally a peptidoglycan degrading enzyme (for example lysozyme, such as egg-hen lysozyme); and/or a mycolic acid degrading enzyme (for example lipase, such as Candida rugosa lipase).

Suitably, the composition may comprise an enzyme having an amino acid sequence as shown in SEQ ID NO: 13, an enzyme having an amino acid sequence as shown in SEQ ID NO: 11, an enzyme having an amino acid sequence as shown in SEQ ID NO: 6, and an enzyme having an amino acid sequence as shown in SEQ ID NO: 3, and optionally a peptidoglycan degrading enzyme (for example lysozyme, such as egg-hen lysozyme); and/or a mycolic acid degrading enzyme (for example lipase, such as Candida rugosa lipase).

Suitably, the arabinogalactan degrading enzymes may be each at a concentration of from about 1 to about 10 μM. Suitably the arabinogalactan degrading enzyme(s) may be at a concentration of about 1 μM, about 2 μM, about 3 μM, about 4 μM, about 5 μM, about 6 μM, about 7 μM, about 8 μM, about 9 μM, about 10 μM, or more. More suitably, the arabinogalactan degrading enzyme(s) may be at a concentration of about 5 μM.

Suitably, the lipase may be at a concentration of from about 0.01% w/v to about 1% w/w, for example about 0.05% w/v, about 0.1% w/v, about 0.2% w/v, or more.

TABLE 4
Concentrations of enzymes used to extract DNA

| Enzyme | Final Concentration | |
|---|---|---|
| Lipase (Sigma-Aldrich L1754) | 0.1% | w/v |
| Lysozyme (Sigma-Aldrich L4919) | 45 | mg/ml |
| HMPREF9455_02479 | 5 | μM |
| HMPREF9455_02470 | 5 | μM |
| HMPREF9455_02481 | 5 | μM |
| BACFIN_08810 | 5 | μM |
| BACFIN_04787 | 5 | μM |

The composition may be freeze-dried or pre-constituted. The composition may be freeze-dried, for example, alone or together with an excipient to increase stability of the enzymes during and/or after freeze-drying. The composition may be freeze-dried in the presence of a cryoprotectant.

In any of the foregoing embodiments, the composition may further comprise a stabilizer. The stabilizer may be a flow agent. The stabilizer may be a cryoprotectant. The cryoprotectant may be glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulphoxide, sodium glutamate, glycine betaine, glycogen, hypotaurine, peptone, polyvinyl pyrrolidone, taurine, mammalian milk oligosaccharides, polysaccharides or a combination thereof.

Suitably, the composition may further comprise a buffer. Merely by way of example the buffer may be HEPES, Tris, phosphate or other similar buffer known in the art.

Suitably, the composition may have a pH of about from 7 to 8. Suitably, the composition may have a pH of about from 7.4 to 7.6, for example about 7.5.

The composition may be contained in a bag, a jar, a capsule or any other kind of container.

The term “about,” as used herein, when referring to measured values, such as amounts, concentrations, temperature, pH, means within ±15% of the specified value, for example ±10%, in some embodiments ±5%, in some embodiments ±1%, in some embodiments ±0.1%.

### A Method of Preparing a Sample, and a Method of Determining the Presence of a Bacterium

In a further aspect, the present invention provides a method for preparing a sample for the detection of a bacterium of the Actinomycetota phylum.

In the context of the present disclosure, a sample is prepared for the detection of a bacterium of the Actinomycetota phylum if some of the components of the polysaccharide cell wall are degraded. Thus, the method comprises contacting the sample with a composition of the invention under conditions that allow for degradation of Actinomycetota bacterial cell wall polysaccharides to occur. The degradation is of arabinogalactan, and optionally of peptidoglycan and/or mycolic acid. It will be appreciated that what components of the bacterial cell wall polysaccharides are degraded will depend upon the enzymes present in the composition.

The term “contacting” as used herein refers to bringing together the composition and sample into physical proximity as to allow the enzymes in the composition to break a linkage in the polysaccharide.

Suitably, the step of “contacting” may last from about 15 minutes to about 120 minutes. For example, the step of contacting may last about 15 minutes, about 20 minutes, about 25 minutes, about 30 minutes, or more. For example, the step of contacting may last about 35 minutes, about 40 minutes, about 45 minutes, about 50 minutes, about 55 minutes, about 60 minutes or more. For example, the step of contacting may last about 70 minutes, about 80 minutes, about 90 minutes, about 100 minutes, about 110 minutes, or about 120 minutes, or more.

Suitably, the step of contacting may be performed at a temperature of from about 33° C. to about 40° C., more suitably at a temperature of about 37° C.

The term “under conditions that allow for degradation” includes any conditions (such as pH, ionic strength, composition concentration, amount of composition used, temperature, and/or incubation time) under which the enzymes in the composition can function to achieve the desired result of degrading Actinomycetota bacterial cell wall polysaccharides. It will be appreciated that the conditions that allow for degradation may depend upon sample type, volume, bacterial species, etc. Methods of determining conditions that allow for degradation will be known to the skilled person and at most will involve routine experimentation to determine such conditions.

Suitably the sample may be selected from the group consisting of a sputum sample, a blood sample, a stool sample, and a urine sample. A “sputum sample” is a sample of mucus that is made available from the lower airways of the lungs. Sputum is particularly useful for microbiological investigations of respiratory infections, such as tuberculosis.

Suitably, the method lyses the bacteria in the sample. Lysis of the bacteria may enable the detection of intracellular biomarkers, such as nucleic acids. However, in some embodiments, lysis is not necessary. For example, when the biomarker is a component of the bacterial cell wall (for example a carbohydrate such as the polysaccharide arabinogalactan) degradation of the bacterial cell wall without lysing the bacteria may be sufficient to prepare the sample for the detection of a bacterium of the Actinomycetota phylum.

Examples of suitable bacteria of the Actinomycetota phylum are provided elsewhere in the present disclosure.

In a further aspect, the present invention provides a method of determining the presence of a bacterium of the Actinomycetota phylum in a sample, comprising:
(i) providing a sample prepared according to the method for preparing a sample as described herein; and
(ii) detecting a biomarker indicative of the presence of the bacterium in the sample.

Suitably, the biomarker may be a nucleic acid (DNA or RNA), protein, carbohydrate (for example a cell wall polysaccharide), and/or a lipid.

Suitably, the step of detecting may comprise isolating the biomarker. Methods of detecting and/or isolating the biomarker will be known to those skilled in the art. It will be appreciated that the methods may depend upon the type of biomarker.

Exemplary isolation techniques known in the art include, for example, use of mechanical force in the form of homogenization, heating/boiling, sonication, bead beating etc.; use of chemical agents such as, but not limited to alkali treatment, high salt treatment, hexadecyltrimethylammonium bromide (CTAB), organic solvents like phenol, chloroform and the like; use of detergents such as sodium dodecyl sulfate (SDS), poly(ethylene oxide) based detergents, polysorbate based surfactants, etc.; use of enzymes like Proteinase K; use of resin or column based DNA isolation such as positively charged ion exchange columns, silica columns and so on; use of magnetic bead based isolation; and the like; and combinations of the above.

In an embodiment where the biomarker is a nucleic acid (such as DNA or RNA), the nucleic acid may be detected using any suitable nucleic acid detection techniques known in the art. Merely by way of example, the nucleic acid may be detected by fluorescent labelling or other reporter based technologies like chemiluminescence, colour based (e.g. HRP system), probe based hybridization, biotin-SAV based system, digoxin/digoxigenin based system, silica based system, assay formats such as bead based assays, chip based or lateral flow assays, and the like, and combinations thereof. Specific detection moieties may be used for detection of DNA, thus allowing for rapid screening tests, such as providing positive or negative results for samples. Further, quantitation can be incorporated into the detection technique.

Suitably the nucleic acid may subjected to an amplification step. An exemplary amplification technique known in the art is polymerase chain reaction (PCR), and variants thereof such as Real-Time PCR (RT-PCR). Alternatively or additionally, the nucleic acids may be amplified using isothermal reactions known to those skilled in the art. Nucleic acids of Mycobacteria isolated from processed sputum sample could also be detected by amplification free DNA sensor or similar technologies known to one skilled in the art. The amplification step provides a higher number of cellular components from a smaller number of cellular components, which facilitates visualizing and diagnosis and provides other advantages.

Suitably, the nucleic acid may further undergo sequencing (for example sanger sequencing, pyrosequencing, and/or next-generation sequencing).

The methods described herein may enable shorter turnaround times from sample collection to determining the presence of bacteria. Due to the use of the compositions of the present invention, cells walls from bacteria of the Actinomycetota phylum may be degraded, and optionally lysed, without any specialized instrumentation (such as a sonicator). This may enable resource poor settings to more promptly and easily diagnose samples with a high degree of sensitivity with diseases such as tuberculosis.

### Pharmaceutical Compositions and Uses Thereof

It will be appreciated that the ability of the enzymes described herein to degrade arabinogalactan may render them useful therapeutics, in particular for bacterial infections caused by bacteria of the Actinomycetota phylum.

Accordingly, in a further aspect, the present invention provides a pharmaceutical formulation comprising a composition of the invention, and a pharmaceutically acceptable excipient, carrier, adjuvant, and/or diluent. For avoidance of doubt, a composition of the invention comprises at least two bacterial cell wall degrading enzymes, wherein at least one of the at least two cell wall degrading enzymes is an enzyme comprising an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or an amino acid sequence which has at least 70% sequence identity thereto. Examples of compositions of the invention are provided herein above.

The term “pharmaceutical formulation”, as used herein, refers to a formulation suitable for use for the treatment of a subject's disease, alone or in combination. Suitably, the disease may be an infection caused by bacteria of the Actinomycetota phylum.

Suitably, the subject may be an animal. The animal may be a mammal or non-mammal. Suitably, the subject may be a mammal such as a human. Suitably, the subject may also be a mammal such as monkey, bear, rat, mouse, mink, rabbit, guinea pig, pig, dog, cat, goat, sheep, horse or cow, or any other animal that may develop an infection caused by bacteria of the Actinomycetota phylum.

As used herein, “pharmaceutically acceptable” refers to a material that is not biologically or otherwise undesirable, i.e., the material may be administered to an individual along with the enzymes described herein without causing any undesirable biological effects or interacting in a deleterious manner with any of the other components of the pharmaceutical composition in which it is contained.

Excipients are natural or synthetic substances formulated alongside an active ingredient (e.g. enzymes or compositions described herein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art. pharmaceutically acceptable adjuvants include but are not limited to aluminum based adjuvants, mineral salt adjuvants, tensoactive adjuvants, bacteria-derived adjuvants, emulsion adjuvants, liposome adjuvants, cytokine adjuvants, carbohydrate adjuvants, and DNA and RNA oligo adjuvants among others.

Diluents are diluting agents. Pharmaceutically acceptable diluents are well known in the art. A suitable diluent is therefore easily identifiable by one of ordinary skill in the art. Merely by way of example, diluents include, but are not limited to, lactose, dextrose, mannitol, and/or glycerol, and/or lubricants and/or polyethylene glycol.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

Suitably, the formulation may for pulmonary, topical, intranasal, intramuscular, intratracheal, subcutaneous, intradermal, transdermal, sublingual intravenous, ocular, rectal, nasal, and/or oral administration. More suitably, the formulation may be for pulmonary delivery and/or topical administration.

Suitably, the formulation may be in the form of a spray, powder, pill, tablet, granules, hard or soft capsule, aqueous solution, alcoholic or oily solution, syrup, emulsion or suspension.

Suitably, the pharmaceutical formulation may comprise a known compound currently used to treat an infection caused by a bacterium of the Actinomycetota phylum. Merely by way of example, such a compound may be an antibiotic. Suitably, the antibiotic may be selected from the group consisting of clarithromycin, azithromycin, rifampin, rifabutin, ethambutol, streptomycin, and amikacin, or a combination thereof.

In a further aspect, the present invention provides a pharmaceutical formulation of the invention for use as a medicament.

Provided herein is also a pharmaceutical formulation of the invention for use in treating a bacterial infection caused by a bacterium of the Actinomycetota phylum.

Further, provided herein is a method of treating a bacterial infection caused by a bacterium of the Actinomycetota phylum in a subject, comprising administering a therapeutically effective amount of the pharmaceutical formulation of the invention to a subject in need thereof.

Suitably, the infection may be a mycobacterial infection.

Suitably, the mycobacterial infection may be caused by a bacterium selected from the group consisting of M. tuberculosis, M. bovis, M. africanum, M. canetti, M. microti. M. smegmatis, M. fortuitum, M. marinum, M. ulcerans, M. paratuberculosis, M. celatum, M. avium, M. leprae, M. lepraemurium, M. intracellulare M. scrofulaceum, M. xenopi, M. genavense, M. kansasii, M. simiae, M. szulgai, M. haemophilum, M. asiaticum, M. malmoense, M. vaccae, M. caprae, M. pinnipe dii and/or M. shimoidei.

Merely by way of example, the infection may result in lung disease, leprosy, lymphadenitis, soft tissue, and/or disseminated disease.

Suitably, the infection may be cause by the bacterium of the Tsukamurellaceae family, such as Tsukamurella paurometabola.

Suitably, the infection may be cause by the bacterium of the Nocardiaceae family such as Nocardia farcinia and/or Nocardia brasiliensis.

The term “administering” as used herein generally refers to the administration of a composition or compound to an individual or system. Suitably routes of administration are described elsewhere in the present disclosure.

The term “therapeutically effective amount,” as used herein, refers to the amount of active compound (i.e. the composition of the invention) that elicits the biological or medicinal response in a tissue, system, or individual that is being sought by a researcher, healthcare provider or individual. The biological or medicinal response in the context of the present disclosure may be a lyses of infection causing bacteria, and thereby reducing or eliminating the infection.

The invention is further described in detail by reference to the following experimental examples. These examples are provided for the purpose of illustration only, and are not intended to be limiting unless otherwise specified.

### EXAMPLES

### Example 1

### 1 Introduction

Until recently almost no enzymes had been reported with activity on mycobacterial arabinogalactan. The first reported enzyme is GIfH1, which is an endo-β-D-galactofuranase from mycobacteria that is specific for cleavage between β-1,5 and β-1,6 linked residues (encoded by Rv3096 in M. tuberculosis) (Shen et al, 2020). Based on reported enzymatic parameters, this enzyme has weak activity and is unlikely to be suitable for mycobacterial lysis. The precise role of this enzyme in mycobacterial biology remains unclear but is suggestive of an active remodelling pathway for this cell wall component. Enzymes with increased activity and other D-Galf specificities have not been described, though evidence that they exist was demonstrated in a large screen recently (Helbert et al, 2019). This report identified enzymes belonging to GH43 sub-groups capable of turning over the synthetic substrate p-nitrophenol-β-D-galactofuranoside (pNP-β-D-Galf).

No enzymes have ever been reported in peer-reviewed literature with endo-β-D-arabinofuranase activity (enzymes that cut within the D-arabinan polymers), although this activity has been reported in crude protein extracts dating back to at least the 1970s (Kotani et al, 1970; Kotani et al, 1971; Xin et al, 1999; Sun et al, 2011). Most recently, endo-activity was described in extracts of M. smegmatis, and through the development of a radiometric assay this activity was suggested to increase upon treatment of cells with ethambutol (Dong, 2006). The identity of these enzymes has not yet been reported.

### 2 Materials and Methods

The key to discovering an efficient enzymatic lysis reagent is access to a diverse range of enzymatic functional units. Here the inventors used the human gut microbiome, as they hypothesised that such a microbiome would give access to a pool of glycan-degrading enzymes. This collection of microorganisms is responsible for the turn-over and use of dietary starch and polysaccharides. Dominated by the Bacteroidetes, this grouping of organisms collectively are amongst the richest known organisms in diversity of glycolytic enzymes. Carbohydrate utilisation by these organisms is typically organised into polysaccharide utilisation loci (PULs), which can be induced upon exposure of the bacterium to a given carbohydrate. Examples of this include mannan derived from yeast, plant cell wall polysaccharides and host glycosaminoglycans for example (Cuskin et al, 2015). The diversity and compartmentalisation of polysaccharide utilisation represents a profound opportunity for enzyme discovery. We have leveraged this remarkable biological diversity by growing a panel of bacteria using mycobacterial arabinogalactan as a sole-carbon source. The enzymes these bacteria used to achieve this were then identified using-omics approaches and provided us with a library of AG-cleaving enzymes.

### 2.1 Bacterial Strains and Growth Conditions

### Bacteroidetes Culture

Bacteroidetes strains were grown in defined minimal media including 5 mg/ml arabinogalactan (see below) under anaerobic conditions at 37° C. For growth curves, strains were cultured in 200 ul volumes in a 96 well plate, and OD monitored using an Epoch plate reader (Biotek).

TABLE 5
Recipe for 50 ml of 2x defined minimal media:

| Component | Mass (mg) or Volume (ml) | |
|---|---|---|
| 10x Bacteroides salts | 10 | ml |
| Balch's vitamins | 1 | ml |
| Trace mineral solution | 1 | ml |
| Purine/Pyrimidine solution | 1 | ml |
| Amino acid solution | 1 | ml |
| Vitamin K₃ (1 mg/ml) | 0.1 | ml |
| FeSO₄ | 0.1 | ml |
| CaCl₂ | 0.1 | ml |
| MgCl₂ | 0.1 | ml |
| Histidine Hematin | 0.1 | ml |
| Vitamin B₁₂ (0.01 mg/ml) | 0.05 | ml |
| L-Cysteine | 100 | mg |

Bring volume to 50 ml with distilled water, pH to 7.2 and syringe-filter through a 0.22 μM membrane filter.

### 2.2 Bacteroidetes Media Solutions & Supplements

### Balch's Vitamins

p-Aminobenzoic acid, 5 mg
Folic acid, 2 mg (Sigma, F7876)
Biotin, 2 mg (Sigma, B4501)
Nicotinic acid, 5 mg (Sigma, N4126)
Calcium pantothenate, 5 mg (Sigma, P2250)
Riboflavin, 5 mg (Sigma, R7649)
Thiamine HCl, 5 mg (Sigma, T4625)
Pyridoxine HCl (vitamin B6), 10 mg
Cyanocobalamin (vitamin B12), 0.1 mg
Thioctic acid (lipoic acid), 5 mg
Distilled Water, 1 L

### Amino Acid Solution

Alanine, Arginine, Asparagine, Aspartic Acid, Cysteine, Glutamic Acid, Glutamine, Glycine, Histidine, Isoleucine, Leucine, Lysine, Methionine, Phenylalanine, Proline, Serine, Threonine, Tryptophan, Tyrosine, Valine
Dissolve 62.5 mg each of the 20 essential amino acids in 250 ml H₂O (5 mg/ml total stock).

### Purine/Pyrimidine Solution

Adenine (Sigma, A2786)
Guanine (Sigma, G11950)
Thymine (Sigma, T0895)
Cytosine (Sigma, C3506)
Uracil (Sigma, U1128)

### Trace Mineral Supplement

EDTA, 0.5 g (Sigma, ED4SS)
MgSO₄·7H₂O, 3 g
MnSO₄·H₂O, 0.5 g
NaCl, 1 g (Sigma, S7653)
FeSO₄·7H₂O, 0.1 g (Sigma, 215422)
CaCl₂), 0.1 g
ZnSO₄·7H₂O, 0.1 g
CuSO₄·5H₂O, 0.01 g
H₃BO₃, 0.01 g (Sigma, B6768)
Na₂MoO₄·2H₂O, 0.01 g
NiCl₂·6H₂O, 0.02 g

### 10× Bacteroides Salts (4 L Recipe)

544 g KH₂PO₄ (Fisher, P284)
35 g NaCl (Sigma, S7653)
45 g (NH₄)₂SO₄ (Fisher, A702)

### Minimal Media Supplements

Vitamin K3 (menadione, Sigma, M5625) stock: 1 mg/ml in ethanol, wrap in foil.
FeSO4 (Sigma, 215422) stock: 0.4 mg/ml in 10 mM HCl, filter sterilized
MgCl2 (Sigma, M8266) stock: 0.1M in water, filter sterilized
CaCl2 (Sigma, C1016) stock: 0.8% (w/v) in water, filter sterilized
Vitamin B12 (Sigma, V2876) stock: 0.01 mg/ml in water, filter sterilized, wrap in foil.
Histidine Hematin (Sigma, H8000 & H3281) solution stock: 1.9 mM Hematin in 0.2M Histidine solution, filter sterilized.

### 2.3 Growth of Mycobacteria

M. smegmatis cultures were grown in tryptic soy broth, M. bovis BCG were grown in 7H9 medium supplemented with ADC, with constant agitation at 37° C. To eliminate clumping, all cultures were supplemented with 0.05% Tween 80 (v/v)

### 2.4 Purification of Mycobacterial Arabinogalactan

Large scale purification of mycobacterial arabinogalactan was achieved by established methodologies. In brief, eight litres of mycobacterial culture was grown to mid exponential phase, cultures were pelleted and resuspended in a minimal volume of phosphate-buffered saline and lysed using an Emulsiflex C3 homogeniser. The lysate was then boiled in a final concentration of 1% sodium dodecyl sulphate (SDS) under reflux. Insoluble material (containing mycolyl-arabinogalactan-peptidoglycan complex) was collected by centrifugation and washed exhaustively with water to remove SDS. The mycolate layer was removed by saponification by 0.5% KOH (w/v) in methanol. Cell wall material was then collected by centrifugation and washed three times with diethyl ether to remove saponified mycolic acids. The phosphodiester linkage between AG and PG was then cleaved by treatment with H₂SO₄ (0.2 M) by bringing the mixture to 85° C. for 15 minutes. The acid was then neutralised by addition of calcium carbonate. The resultant solubilised arabinogalactan was dialysed exhaustively against water and lyophilised (yield=˜22.5 mg/L).

### 2.6 Purification of Mycobacterial Lipoglycans

One litre of mycobacterial culture was grown to mid exponential phase and pelleted as previously. The pellet was resuspended in 20 mL PBS+0.1% Tween-80, chilled and lysed by bead-beating. Lysate was transferred to a Teflon-capped glass tube and vortexed with an equal volume of citrate buffer saturated with Phenol, and heated to 80° C. for three hours, vortexing every hour. A biphase was generated by centrifugation at 2000 RPM at 10° C., and the upper aqueous phase transferred to a fresh glass tube and hot phenol wash repeated twice more. The resultant protein free glycan mixture was dialysed exhaustively against tap water overnight to remove trace phenol and lyophilised, yielding 34 mg of crude lipoglycans (LAM, LM, PIMS) per litre of culture.

2.7 Pseudomonas aeruginosa Pilin Oligosaccharide Extraction.

Modified from: Voisin S, Kus J V, Houliston S, St-Michael F, Watson D, Cvitkovitch D G, Kelly J, Brisson J R, Burrows L L. Glycosylation of Pseudomonas aeruginosa strain Pa5196 type IV pilins with mycobacterium-like alpha-1,5-linked d-Araf oligosaccharides. J Bacteriol. 2007, 189(1): 151-9. doi: 10.1128/JB.01224-06. PMID: 17085575

Pilins were purified as described by Burrows and colleagues, with some modifications. Briefly, Pseudomonas aeruginosa Pa5196 were streaked out in a grid pattern onto LB agar plates and grown for 24 hours at 37° C. Cells were then scraped from all plates using sterile cell scrapers and resuspended in 4 ml of sterile phosphate-buffered saline (pH 7.4) per plate.

Pili were then sheared by vigorous vortexing of resuspended cells for 2 minutes. This suspension was then centrifuged for 5 minutes at 6000×g. The supernatant was transferred to high-speed centrifuge tubes and centrifuged for 20 minutes at 20,000×g to ensure all cells were pelleted. Supernatants were transferred to fresh tubes and MgCl₂ was added to give a final concentration of 100 mM. Following inversion of these tubes to ensure mixing, samples were incubated at 4° C. overnight, allowing precipitation of sheared proteins. Samples were then centrifuged for 20 minutes at 20,000×g, yielding a precipitate smudge which was resuspended in 50 mM NH₄HCO₃, pH 8.5 and transferred to fresh Eppendorf tubes. This solution was then dialysed into the same buffer to eliminate excess MgCl₂.

Bradford assays were then performed to assay the mass of protein in the sample, followed by digestion of the intact protein pilins using proteinase K in a 50:1 pilin to enzyme ratio by mass for 24 hours in the presence of 2 mM CaCl₂). Glycans were then purified from digested proteins using porous graphitised carbon (PGC) chromatography, where sugars were eluted from the column using a twofold increasing concentration series of a butan-1-ol:H2O mix from 1:32 to 1:1 using 1 mL elutions. Thin-layer chromatography (TLC) of eluates showed various oligomers of arabinan present in all fractions, all of which were subsequently used as substrates for potential arabinofuranosidases.

### 2.8 Molecular Biology

Genes were amplified by PCR from genomic DNA (B. finegoldii, D. gadei) or synthesised by Twist Biosciences (mycobacteria, Nocardia and Mycosynbacter) into pET28a expression vector, including a hexa-histidine tag to facilitate affinity purification.

### 2.9 Purification of Rv3707c

For production of Rv3707c, an aliquot of competent BL21 DE3 E. coli was transformed with pET28a_3707_P30M and plated on LB agar supplemented with kanamycin. One plate of bacteria was scraped to inoculate one litre of autoinduction media. The bacteria were incubated at 37° C. until OD₆₀₀=0.6, whereupon the flasks were cooled on ice and incubated overnight at 20° C. After induction, the cultures were pelleted at 4000×g for 25 minutes (C). pellets were resuspended in sterile PBS and pelleted at 5000×g for 10 minutes. The supernatant was removed and pellets were snap frozen in liquid nitrogen and stored at −20° C. until preparation.

Rv3707c_P30M was purified by suspension of one pellet in cold lysis buffer (25 mM HEPES pH 8; 400 mM NaCl; 5% glycerol; 50 mM L-arginine; 50 mM L-glutamic acid; 0.5 mM beta-mercaptoethanol). 10 ug/mL deoxyribonuclease I from bovine pancreas (Sigma-Aldrich) was added to cell slurry and incubated for 30 minutes. Cells were lysed by three passages through a French pressure cell. Insoluble debris was then pelleted by centrifugation. The supernatant was then processed by immobilised metal affinity chromatography (IMAC) on a drip column containing 2 mL bed volume of Ni-NTA resin (ThermoFisher Scientific). After loading the lysate, the column was washed with 80 mL of lysis buffer, then eluted with an imidazole gradient of 50, 100, 250, and 500 mM. Protein-containing fractions were pooled and dialysed exhaustively against three litres of dialysis buffer (25 mM HEPES pH 8; 400 mM NaCl; 5% glycerol; 50 mM L-arginine; 50 mM L-glutamic acid; 2 mM dithiothreitol).

### 2.10 Gut Bacteria Protein Expression and Purification

Recombinant proteins were produced in competent E. coli Tuner cells (Novagen) using pET28a vectors. Cells were grown in LB media at 37° C., and expression was induced with 0.2 mM IPTG for 16 hours at 16° C. Sonication was used to lyse cells in 20 mM Tris, pH 8.0, 200 mM NaCl, followed by purification of recombinant proteins from clarified lysate.

Enzymes were purified using immobilised metal affinity chromatography on cobalt TALON resin. Proteins were dialysed into 20 mM HEPES, pH 8.0, 150 mM NaCl buffer using size 5 dialysis tubing (Medicell). For crystallography they were purified further via size-exclusion chromatography (HiLoad 16/600 Superdex 200, GE Healthcare) in 20 mM Hepes, pH 8.0, 150 mM NaCl. Purified proteins were then concentrated using 10,000 MWCO centrifugal filter units (Sartorius) to desired concentration.

### 2.11 Purification of MYCMA_14050 and O31_017420

One plate of BL21-DE3 transformed with an appropriate plasmid was used to inoculate 1 L Terrific Broth supplemented with kanamycin. The culture was grown to an OD₆₀₀ of 0.6 and induced with 0.25 mM IPTG and grown overnight at 20° C. After harvest of biomass as in purification of Rv3707c, cell pellets were resuspended in a buffer containing 25 mM HEPES; 400 mM NaCl, lysozyme, and DNAse I). Subsequent purification steps were identical to those in Rv3707c, but in the above, simpler buffer, omitting lysozyme and DNAse I.

### 2.12 Thin-Layer Chromatography (TLC)

Various concentrations (as indicated) of purified proteins were incubated with various concentrations (as indicated) of a range of select substrates for 16 hours at 37° C. to ensure reaction completion (unless otherwise indicated). Using TLC plate aluminium foils (Silicagel 60, 20×20, Merck) which were cut to the desired size (minimum height of 10 cm), these reactions samples were spotted (6 μl, unless otherwise indicated) and allowed to dry. TLC plates were run (twice) in solvent (1-butanol/acetic acid/water 2:1:1 (v/v)). Plates were then removed and dried before visualisation of sugars was obtained via immersion of TLC plate in Orcinol stain. Plates were dried and developed through heating between 50° C. and 80° C.

2.13 Lon Chromatography with Pulsed Amperometric Detection (IC-PAD)

Oligosaccharides from enzymatic polysaccharide digestion were analysed using a CARBOPAC PA-300 anion exchange column (ThermoFisher) on an ICS-6000 system. Detection enabled by PAD using a gold working electrode and a PdH reference electrode with standard Carbo Quad waveform. Buffer A—100 mM NaOH, Buffer B—100 mM NaOH, 0.5 M Na Acetate. Each sample was run at a constant flow of 0.25 ml/min for 100 minutes using the following program after injection: 0-10 min; isocratic 100% buffer A, 10-70 min; linear gradient to 60% buffer B, 70-80 min; 100% buffer B. The column was then washed with 10 mins of 500 mM NaOH, then 10 min re-equilibration in 100% buffer A. L-arabino-oligosaccharides obtained commercially (Megazyme) were used as standards at a concentration of 25 μM. Data were processed using Chromeleon™ Chromatography Management System V.6.8. Final graphs were created using GraphPad Prism 8.0.1.

2.14 Mycobacterial gDNA Extraction

Each enzyme was concentrated to stock solution of 100 μM in 20 mM HEPES, pH 8.0, 150 mM NaCl. Mycobacteria species were cultured to an OD₆₀₀ of 0.7, and 700 μl of culture pelleted for 3 minutes at 13000×g in a 1.5 ml Eppendorf. The pellet was resuspended in in 225 μl Gram-positive Lysis Buffer* (Sigma-Aldrich) containing 45 mg ml⁻¹ lysozyme. Each enzyme was added to a final concentration of 5 μM in a final volume of 315 μl. Reactions were incubated in a thermal mixer (Eppendorf ThermoMixer® C) preheated to 37° C. for 15-120 minutes at 2000 RPM. Following the incubation the GenElute™ Bacterial Genomic DNA Kit protocol was followed. *Lysis buffer is likely to be 10 mM Tris-HCl, pH 8.0, with 0.1 M NaCl, 1 mM EDTA, and 5% [w/v] TRITON. X-100 (https://www.sigmaaldrich.com/GB/en/technical-documents/technical-article/protein-biology/protein-lysis-and-extraction/lysing-enzymes).

### 3 Results

### 3.1 AG-Growth Screen

Growth of gut bacterial species on arabinogalactan (AG) from M. smegmatis identified several species capable of metabolising it as a sole carbon source (FIG. 3). As evidenced from analysis of extracellular degradation products (FIG. 3), some of these organisms are specialists for hydrolysing the galactan whereas others were capable of degrading both D-arabinan and D-galactan (FIG. 3).

### 3.2 Galactan Degrading Enzymes

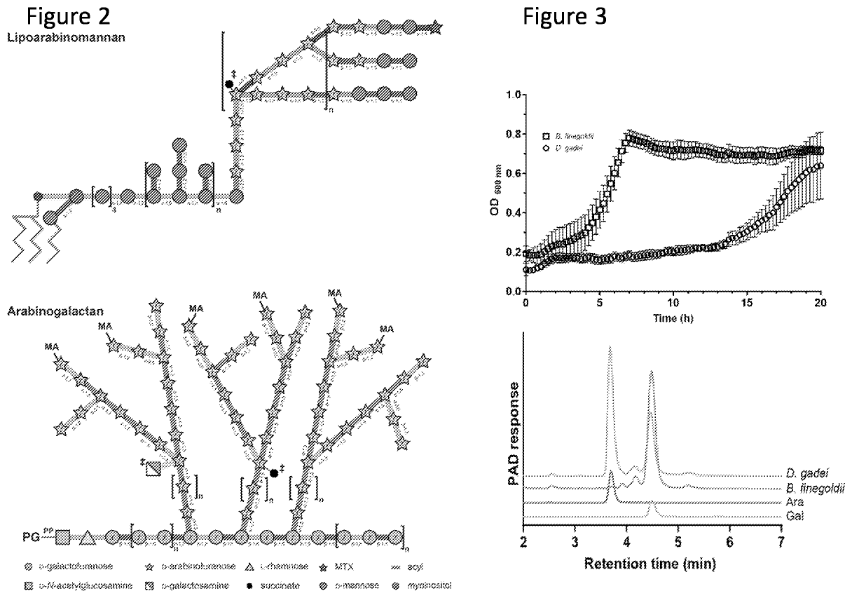

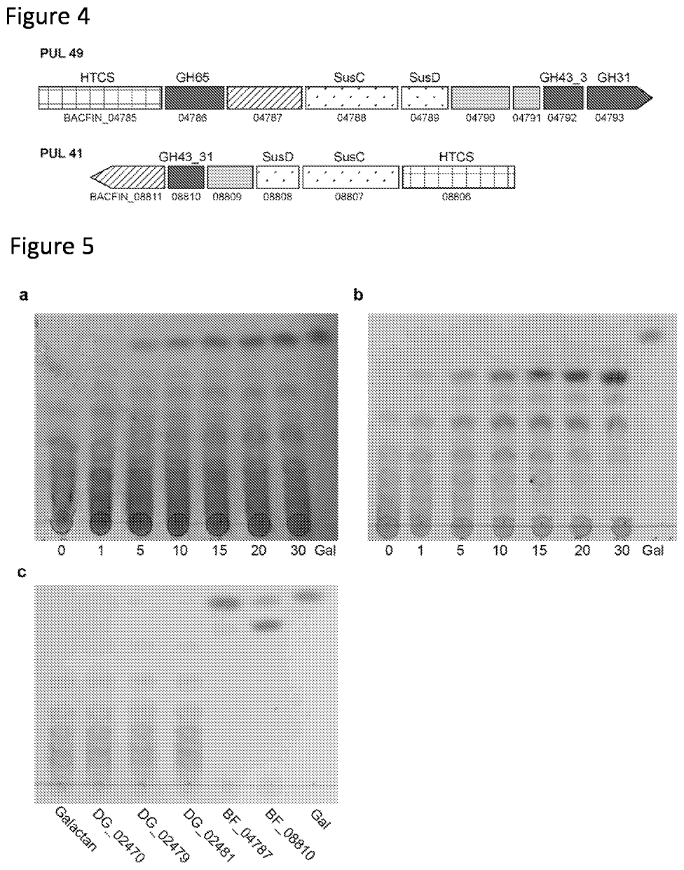

Several Bacteroides species including Bacteroides finegoldii DSM 17565 (FIG. 3) are able to grow on AG but only release galactose during growth and therefore are only able to target the galactose component of the polysaccharide. Helbert et al (2019) identified previously uncharacterised GH43 subfamilies active on pNP-β-D-Galf, including GH43_3, 8, 31, 32, 34 and 37. We identified two PULs in the B. finegoldii genome which contain GH43_3 and GH43_31 (FIG. 4) subfamily members as well as several proteins of unknown function (FIG. 4). We cloned and expressed these proteins and tested their activity on pNP-β-D-Galf, AG from M. smegmatis, and unsubstituted D-galactan from a Corynebacterium glutamicum ΔubiA mutant. In C. glutamicum, the arabinan and mycolate are not essential under certain growth conditions as they are in mycobacteria (Alderwick et al, 2005), so the ΔubiA strain can be used to produce linear galactan as shown in FIG. 2.

The GH43_31 enzyme (BACFIN_08810) was active on pNP-β-D-galactofuranoside and D-galactan, as were the proteins of unknown function BACFIN_08811 and BACFIN_04787 (FIG. 5). Interestingly, these two proteins share 66% identity and therefore are two founding members of a new glycoside hydrolase family. The new family (GalFH) are exo-galactofuranosidases and can hydrolyse both the β-1,5 and β-1,6 bonds in mycobacterial galactan. The GH43_31 family enzyme (BACFIN_08810) displays endo activity and releases galactobiose from mycobacterial galactan, which can be hydrolysed by BACFIN_04787 or 08811 resulting in complete hydrolysis of galactan (FIG. 5). These enzymes collectively provide us with a tool-kit for total D-galactan degradation.

### 3.3 Arabinan-Degrading Enzymes

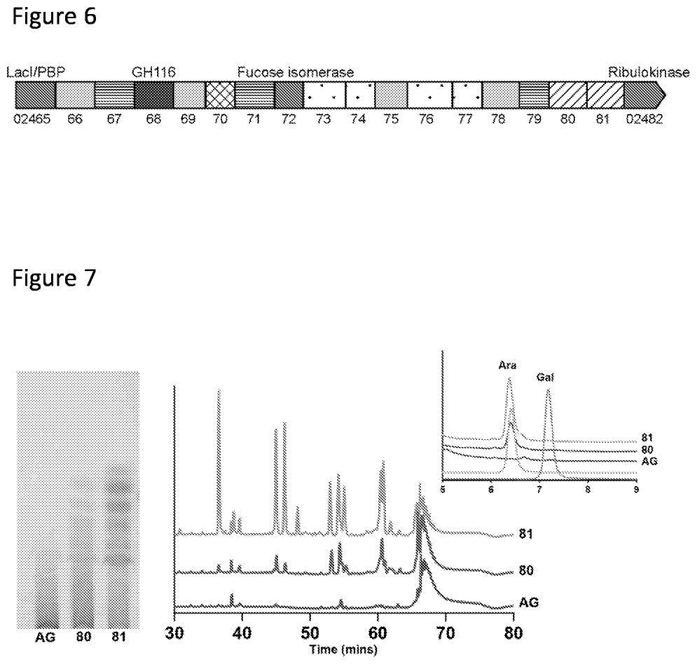

Having D-galactan degrading enzymes in-hand, we next sought to identify enzymes capable of degrading D-arabinan. We observed during growth of Dysgonomonas gadei on AG (FIG. 3) that, in contrast to B. finegoldii, D. gadei released arabinose during growth as well as galactose. We prepared pure mycobacterial D-arabinan by degrading the D-galactan backbone using the enzymes identified above, and conducted a proteomic analysis of D. gadei cells at mid-exponential phase to identify abundant proteins likely involved in this mode of growth. A PUL was identified (FIG. 6), containing the SusCD transporters typical of Bacteroidetes PULs, one GH116 family member and some likely metabolic genes. Half of the genes (9) encode proteins of unknown or unannotated function. We have cloned, expressed, purified, and tested the activity of the proteins of unknown function to identify if any were able to degrade D-arabinan. We have identified three families of D-arabinan degrading enzymes (FIG. 6).

### DUF4185 Family

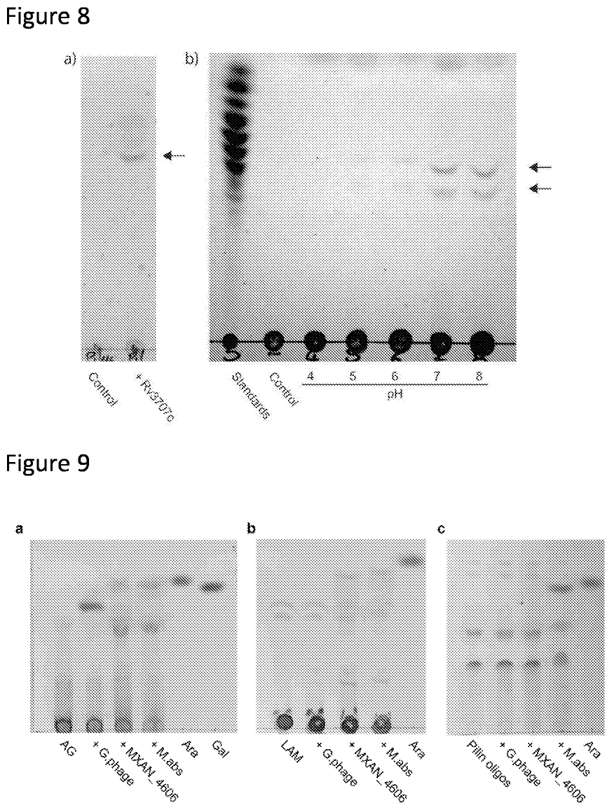

D. gadei 02480 and 02481 contain a domain of unknown function annotated as DUF4185 in the Pfam database. Bioinformatic analysis of this family identified potential homologs in many species of bacteria, fungi and phage suggesting possible functional diversity within the family. Indeed, many CAZy GH families encode enzymes with distinct substrate specificities. To test this, alongside the two proteins from D. gadei we expressed homologs from Mycobacterium tuberculosis, Mycobacterium abscessus, Myxococcus xanthus and Gordonia phage GMA6. Surprisingly, all of these displayed the ability to hydrolyse arabinogalactan from M. smegmatis, releasing D-arabino-oligosaccharides albeit with varied substrate specificity (FIGS. 7, 8, 9). We further tested the activity of the enzymes on pili from Pseudomonas aeruginosa PA7 which is known to be glycosylated with α-1,5-D-arabinofuranose oligosaccharides thereby demonstrating their activity on this glycosidic linkage (FIGS. 8 and 9) (Voisin, 2007). Furthermore, the enzymes showed no activity against pure D-galactan (FIG. 5c) confirming their restricted activity.

### DUF2961 Family

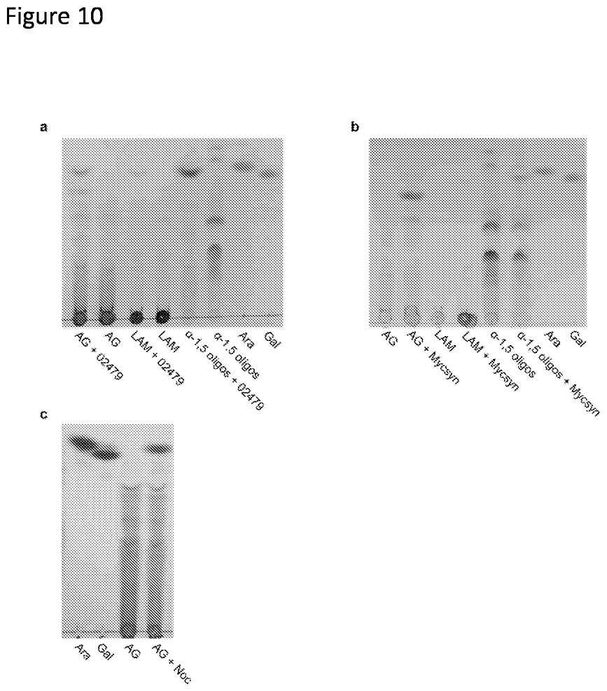

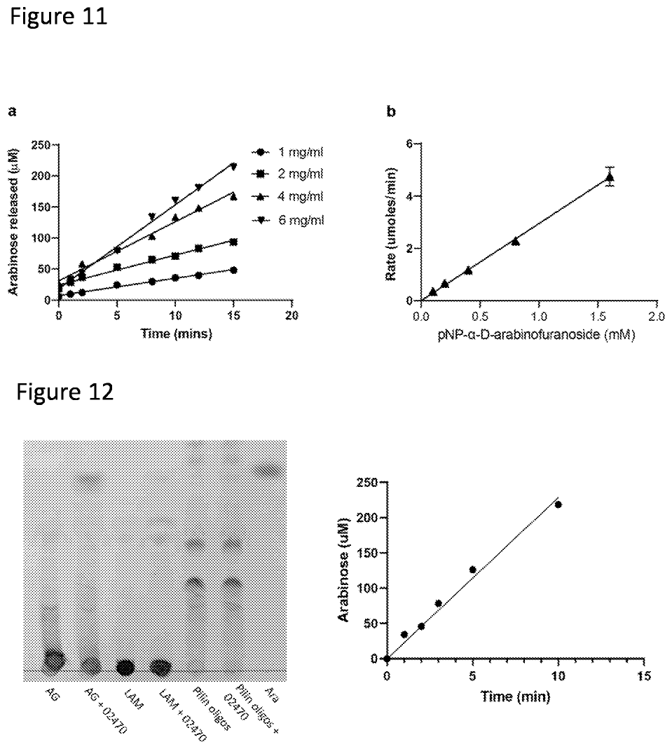

There are three copies of genes which encode DUF2961-containing proteins in the D. gadei D-arabinan PUL; HMPREF9455_02467, 71 and 79. These, in addition to distantly related enzymes (O31_017420) from Nocardia brasiliensis ATCC 700358 and Candidatus Mycosynbacter amalyticus JR1 were cloned and expressed and tested against AG and LAM from M. smegmatis, and α-1,5-D-arabinofuranose oligosaccharides from pili as described above. All showed release of arabinose monosaccharide from AG and pilin oligosaccharides, verified by TLC and HPAEC-PAD, indicating they comprise a family of D-arabinofuranosidases (FIGS. 10 and 11). They are all active against the synthetic substrate pNP-α-D-arabinofuranoside, synthesised by collaborator Prof. Spencer Williams, but not pNP-β-D-arabinofuranoside or pNP-α-L-arabinofuranoside, confirming they are specific for α-D-arabinose (FIG. 11). They also show no activity against D-galactan.

### Arabinofuranosidase HMPREF9455_02470

The protein encoded by 02470, (FIG. 6), is annotated as ‘six-hairpin glycosidase-like’ protein, but showed no sequence similarity to known or characterised glycoside hydrolases. We cloned, expressed and purified 02470, and incubated with AG, showing release of arabinose monosaccharide on both TLC and HPAEC-PAD (FIG. 12). In contrast to the DUF2961 family, 02470 was not active on either α- or β-forms of pNP-D-arabinofuranoside, suggesting it has specificity for more than the −1 subsite. 02470 is very weakly active on α-1,5-arabino-oligosaccharides from P. aeruginosa pilins, so likely prefers either branched substrates or α-1,3 linkages (FIG. 12).

### 3.4 Complete Degradation of Mycobacterial Arabinogalactan

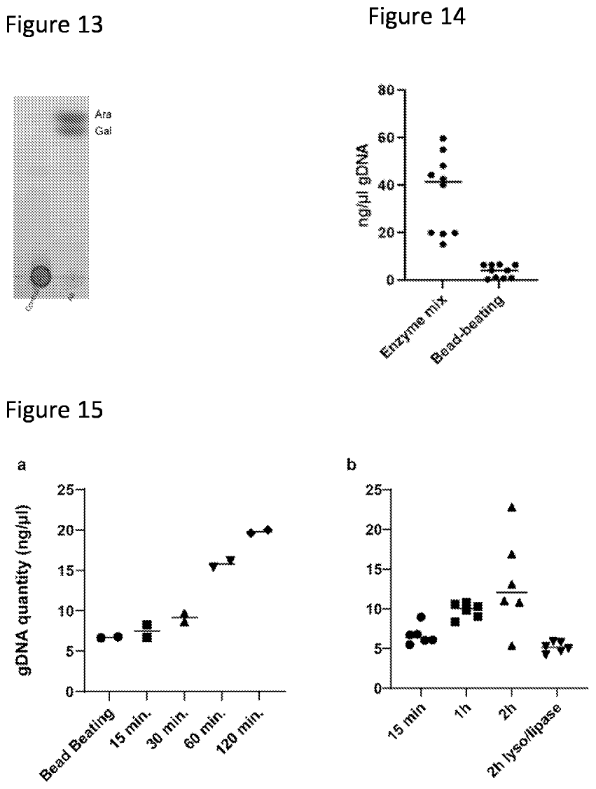

Given the essentiality of AG to M. tuberculosis and other mycobacteria, we investigated whether a combination of the enzymes described here was sufficient to completely degrade AG. To achieve this we combined DG_02470, DG_02479, DG_02480, DG_02481, BACFIN_04787 and BACFIN_00810 with purified arabinogalactan and analysed the reaction products by TLC (FIG. 13). Based on these results it was evident that we had established a consortia of enzymes capable of total AG degradation.

### 3.5 Lysis of Mycobacteria

Given that we now had a cocktail of enzymes that could completely degrade the arabinogalactan of mycobacteria, we sought to pair them with enzymes known to degrade peptidoglycan and mycolic acids. LysB is a lysin encoded by mycobacteriophage that has previously been shown to cut the bond between AG and mycolate. Characterised homologs of this enzyme however suffer from solubility and stability limitations and so are not suitable for deployment in a mycobacterial lysis reagent. Given the structural similarity between the mycolate-AG linkage and tri-acyl glycerides, we included a commercially available lipase from Candida rugosa. Similarly, we have included hen-egg white lysozyme in our lysis cocktail based on its common use in lysis kits for other bacteria. To determine whether enzymes described above could be used as a lysis reagent for DNA extraction, approximately 10⁸ CFU of M. smegmatis were used for each replicate. The enzyme combination listed in Table 4 was incubated with M. smegmatis cells in the Sigma GenElute Kit Gram positive lysis buffer for 2 h at 37° C. in a thermal mixer, shaking at 2000 rpm.

After the 2 h incubation the Sigma GenElute kit and reagents were used according to manufacturer's instructions. The eluted genomic DNA concentration was quantified using the Qubit dsDNA High Sensitivity Assay kit (ThermoFisher) (FIG. 14), showing enzymatic lysis is more effective than bead-beating at releasing genomic DNA from M. smegmatis.

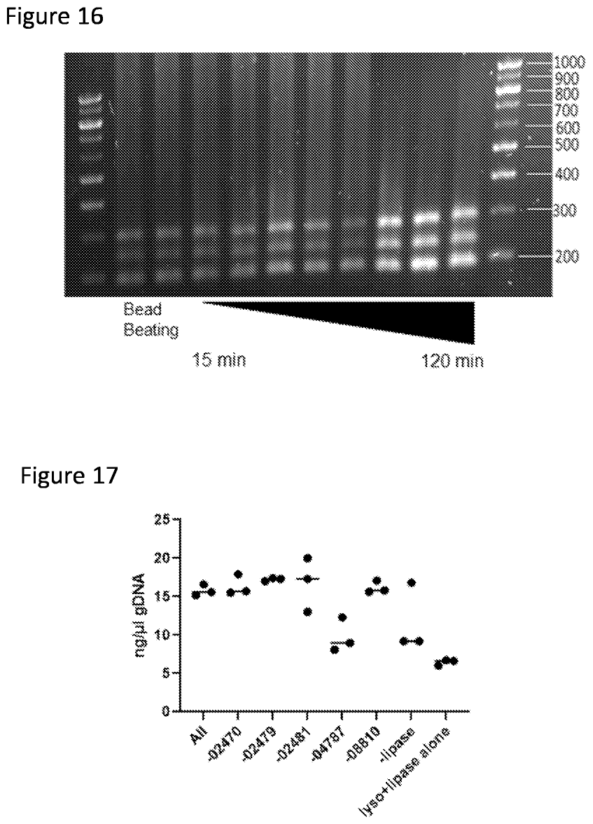

The optimal time of enzyme lysis step was also studied, from 15 minutes to 2 hours for both M. smegmatis and M. bovis BCG (FIG. 15). In addition to the quantification of genomic DNA, a set of multiplex primers was designed to amplify three regions from the M. smegmatis genome (Table 6, FIG. 16). These results show that a 15-minute enzymatic lysis step is equivalent to bead-beating in terms of genomic DNA yield, and is sufficient to provide template for PCR amplification.

TABLE 6
Amplicons and primers used for assessing genomic DNA preparations

| | Fragment | | |
|---|---|---|---|
| Gene | size | Forward primer | Reverse primer |
| Msmeg_0157 | 295 bp | ATCTGCCGCTACAACCTTC | TGCCCTTGGGATTGAGATG |
| | | (SEQ ID NO: 43) | (SEQ ID NO: 44) |
| Msmeg_0902 | 243 bp | CCTTTCCGACGACTTCTTCC | TTCCTCGACAGTGTCAGTCC |
| | | (SEQ ID NO: 45) | (SEQ ID NO: 46) |
| Msmeg_3943 | 192 bp | ACGATTCGTCCTATCACGCC | ACAACACCACGACCTTTGCC |
| | | (SEQ ID NO: 47) | (SEQ ID NO: 48) |

Once we had established an effective cocktail, we sought to investigate which enzymes were critical to lysis, by conducting replicate experiments in which each novel enzyme or lipase were excluded from the cocktail (FIG. 17). For this 2 hour lysis, a lack of either 04787 or lipase was shown to affect total gDNA yield. For more rapid lysis, more enzymes might be necessary.

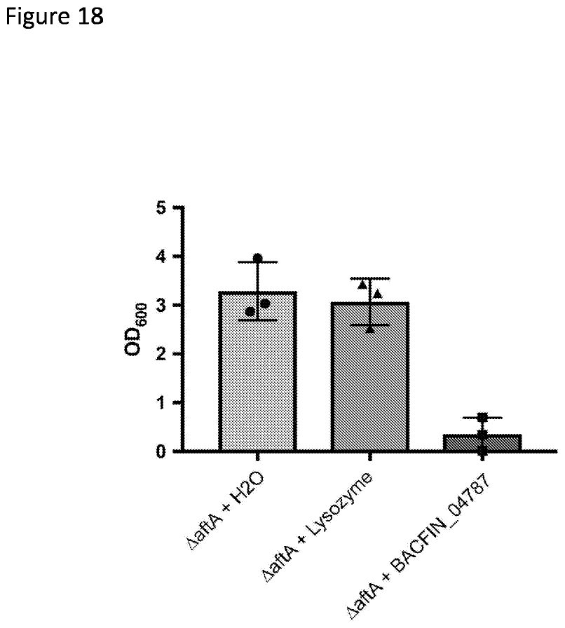

To determine if the enzymes were contributing to lysis we sought to study their potential to kill model bacteria. Corynebacterium glutamicum is a closely related species to mycobacteria. It possesses the same general cell wall structure, however unlike mycobacteria it can remain viable without the mycolate or D-arabinan layers. One way to achieve this is through deletion of the gene encoding the major D-arabinosyl-transferase AftA (Alderwick et al, 2006). A C. glutamicum ΔaftA strain lacking arabinan and mycolate was incubated with either lysozyme or novel galactofuranosidase BACFIN_04787 for 18 h, and lysis measured by drop in optical density (FIG. 18). No effect of lysozyme was evident but incubation with BACFIN_04787 led to an approximate 8-fold drop in OD, indicating lysis of the bacterium.

### 4 Discussion

Clinical microbiology increasingly relies upon high throughput solutions for microbe identification. This typically requires the use of robotics platforms, and in the context of infectious agents these ideally should have as few handling steps as possible. These demands create a substantial technical hurdle for mycobacterial diagnostics, where a simple chemical or enzymatic lysis technology that does not damage DNA is not available. Current solutions to this problem involve mechanical means such as bead-beating, sonication, or pyrolysis. These solutions are sub-optimal and each of them requires instrumentation which can be limited in low-resource settings and requires maintenance and periodic replacement. Bead-beating and sonication are particularly problematic because both have the potential to generate infectious aerosols. Even pyrolysis could lead to aerosol generation should the high-temperature sample be exposed to the user. In this context we sought to identify a collection of enzymes which would enable one-pot lysis of mycobacteria, thereby liberating genomic DNA for downstream diagnostic technologies. Our flexible enzymatic mixture includes enzymes from four major classes including enzymes that cleave peptidoglycan, mycobacterial D-galactan, D-arabinan and lipid linkages.

This first-in-class mycobacterial enzymatic lysis reagent provides a tremendous opportunity. This is because the lysis cocktail described herein is not limited to the generation of genomic DNA for diagnostics. The lysis caused by these enzymes will release other diagnostically relevant molecules that can be detected using technologies like enzyme-linked immunosorbent assays, loop-mediated isothermal amplification, or lateral flow tests. Furthermore, this combination of enzymes could be deployed as an enzybiotic for the treatment of diseases caused by mycobacteria and related organisms. This lysis reagent could also be deployed in any context where arabinogalactan-containing species are unwanted colonisers. For example, they may have application as disinfectants in industrial processing contexts where C. glutamicum is used in the production of amino acids or other products. Waste-water fouling is also often caused by organisms related to mycobacteria such as Gordonia species and this lysis reagent could be used as a bio-control tool in that context. Finally the individual enzymes described herein could be employed in the generation of diagnostic biomarkers in their own right.

In summary, the inventors have developed a novel and flexible technology that surpasses many current limitations in mycobacterial diagnostics. These enzymes hold tremendous potential for expanding the ability to detect, treat and contain bacteria that have arabinogalactan as a component of their cell walls.

### Example 2

Materials and Methods—Lysis with Enzyme Cocktail Vs. Bead-Beating

Bacterial culture grown to ˜OD600 of 0.7 was pelleted in 700 μl aliquots. For enzymatic lysis, pellets were resuspended in 200 μl of Sigma Aldrich Gram positive lysis buffer containing 45 ml/ml lysozyme and 10 mg/ml lipase. Cocktail enzymes (SEQ ID NO:1, 3, 6, 11, and 13) were added to final concentration of 5 M each. Tubes were incubated in a thermal mixer at 37° C., 2000 rpm for 120 minutes. After incubation the Sigma Aldrich GenElute™ Bacterial Genomic DNA Kit protocol was followed.

For bead-beating, pellets resuspended in 700 μl of phosphate-buffered saline were added to vials with silica beads and beaten in a bead-beater at 3500 rpm for 1 minute. Supernatant was transferred to a clean Eppendorf before continuing with GenElute protocol. Purified genomic DNA concentrations were measured using the Qubit Fluorimeter, and Qubit dsDNA high sensitivity dye.

Concentrations were adjusted to 50 ng gDNA in 9 μl water, and 1 μl of Nanopore Rapid barcoding kit was added before sequencing on Nanopore MINION. Analysis of reads was produced using NanoStat (https://github.com/PeterJBurke/Nanostat) providing longest read, N50 and total reads.

### Results

TABLE 7
Representative whole-genome sequencing data. All samples were prepared using
commercial genome preparation kits, with preceding lysis following either our
cocktail-based approach for 2 hours or using the gold-standard of bead-beating.
The concentration of the DNA available for Nanopore sequencing is listed followed
by the N50 summary statistic, and longest read observed. These two metrics are also
presented as a ration of Cocktail/Bead beating (N50_{C}/N50_{B} or LR_{C}/LR_{B} respectively).

| | | Qubit | | | Longest | |
|---|---|---|---|---|---|---|
| Species | Conditions | (ng/uL) | N50 | N50_{C}/N50_{B} | Read | LR_{C}/LR_{B} |
| Tsukamurella | Bead | 2.94 | 874 | 6.68 | 12951 | 3.35 |
| paurometabola | Cocktail | 29.8 | 5841 | | 43430 | |
| Nocardia farcinia | Bead | 16.8 | 2635 | 2.39 | 26488 | 1.80 |
| | Cocktail | 10.7 | 6293 | | 47644 | |
| M. avium subsp. | Bead | 6.4 | 1937 | 7.14 | 33454 | 1.72 |
| paratuberculosis | Cocktail | 9.86 | 13821 | | 57655 | |
| M. marinum | Bead | 1.2 | 1523 | 4.19 | 14574 | 4.26 |
| | Cocktail | 3.16 | 6384 | | 62132 | |
| Nocardia | Bead | 1.16 | 2415 | 1.18 | 50491 | 1.18 |
| brasiliensis | Cocktail | 2.12 | 2848 | | 59545 | |
| M. bovis BCG | Bead | 8.54 | 1736 | 2.64 | 18496 | 1.41 |
| | Cocktail | 9.62 | 4581 | | 26109 | |

As shown in Table 7, the enzyme cocktail consistently performed as well or better than bead-beating.

SEQUENCES

SEQ ID NO: 1
MVNYVPENISASISLKVPGNVAERYSLNLQQLGNNRSSYLLEASESIPVTITQRVADVNGKL
RVNVCITALEDIYFNYSQRLSTGYRHDDCQFYMPGFWYRRNLRSPKDAPSFHSSDSWLVR
EDRLSAPLTGVYSEKEKHFVTVSRIDKFAEEALSTHREGEIILSGKTSLGFTGFENQDGITAL
SFGFPYREAPKSYIRKLTLAPAVEAFQFLKKGETVLLTWEIQENKADDYSDFIRHAWEYSYD
TYGPKPVETPYSITDMKQVLSGFFISSFVEGHPLVYNSGIHLMTENCQSNGQAEVGFIGRVL
LNAFNAWEYGHQSDREDLKANSMKVFDSYLKNGFTPVGFFKESVDFDKGYEDPVHSIRRQ
SEGIYAMLHFLAYEKENGRRHPEWEQKMKNMLDILLRLQQADGSFPRKFRDDFTIVDTSGG
STPSATLPLVMGYKYFKDKRYLASAKQTADYLEKVLISKADYFSSTLDANCEDKEASLYAAT
ATYYLSLITKGDEHRHYADLTKKAAYFALSWYYVWDVPFAQGQMLGDIGLKTRGWGNVSV
ENNHIDVFVFEFASVLRWLSKEYAEPRFSQFAEVISTSMRQLLPHEGHLCGVAKPGYYPEV
VQHTNWDYGKNGKGYYNDIFAPGWTVASLWELFTPGRAEYFLK
SEQ ID NO: 15
MVNYVPENISASISLKVPGNVAERYSLNLQQLGNNRSSYLLEASESIPVTITQRVADVNGKL
RVNVCITALEDIYFNYSQRLSTGYRHDDCQFYMPGFWYRRNLRSPKDAPSFHSSDSWLVR
EDRLSAPLTGVYSEKEKHFVTVSRIDKFAEEALSTHREGEIILSGKTSLGFTGFENQDGITAL
SFGFPYREAPKSYIRKLTLAPAVEAFQFLKKGETVLLTWEIQENKADDYSDFIRHAWEYSYD
TYGPKPVETPYSITDMKQVLSGFFISSFVEGHPLVYNSGIHLMTENCQSNGQAEVGFIGRVL
LNAFNAWEYGHQSDREDLKANSMKVFDSYLKNGFTPVGFFKESVDFDKGYEDPVHSIRRQ
SEGIYAMLHFLAYEKENGRRHPEWEQKMKNMLDILLRLQQADGSFPRKFRDDFTIVDTSGG
STPSATLPLVMGYKYFKDKRYLASAKQTADYLEKVLISKADYFSSTLDANCEDKEASLYAAT
ATYYLSLITKGDEHRHYADLTKKAAYFALSWYYVWDVPFAQGQMLGDIGLKTRGWGNVSV
ENNHIDVFVFEFASVLRWLSKEYAEPRFSQFAEVISTSMRQLLPHEGHLCGVAKPGYYPEV
VQHTNWDYGKNGKGYYNDIFAPGWTVASLWELFTPGRAEYFLKLEHHHHHH
SEQ ID NO: 29
MNFKCLFCGALITLYVGVAEAVNYVPENISASISLKVPGNVAERYSLNLQQLGNNRSSYLLE
ASESIPVTITQRVADVNGKLRVNVCITALEDIYFNYSQRLSTGYRHDDCQFYMPGFWYRRNL
RSPKDAPSFHSSDSWLVREDRLSAPLTGVYSEKEKHFVTVSRIDKFAEEALSTHREGEIILS
GKTSLGFTGFENQDGITALSFGFPYREAPKSYIRKLTLAPAVEAFQFLKKGETVLLTWEIQEN
KADDYSDFIRHAWEYSYDTYGPKPVETPYSITDMKQVLSGFFISSFVEGHPLVYNSGIHLMT
ENCQSNGQAEVGFIGRVLLNAFNAWEYGHQSDREDLKANSMKVFDSYLKNGFTPVGFFKE
SVDFDKGYEDPVHSIRRQSEGIYAMLHFLAYEKENGRRHPEWEQKMKNMLDILLRLQQAD
GSFPRKFRDDFTIVDTSGGSTPSATLPLVMGYKYFKDKRYLASAKQTADYLEKVLISKADYF
SSTLDANCEDKEASLYAATATYYLSLITKGDEHRHYADLTKKAAYFALSWYYVWDVPFAQG
QMLGDIGLKTRGWGNVSVENNHIDVFVFEFASVLRWLSKEYAEPRFSQFAEVISTSMRQLL
PHEGHLCGVAKPGYYPEVVQHTNWDYGKNGKGYYNDIFAPGWTVASLWELFTPGRAEYFLK
SEQ ID NO: 2
IEYTPANVSASIALKVPGNEAVRYPLEFRKLRKERFDYQLTATESLPVLIYQKVEGGETNKRI
TLFITATENIYFNYGEQVKSGACHDDCLFYMPGFWYRRNLRSPKEAPSFHTSDSWVVRED
RLSTPMTTIFDEKNGKSFSVARIDKFESDALTTHKEGEVILSGTTSIGYTGFENCNGTATLSF
GYPYKEAPKTYIRKLTLAPSVEAYQFLGKGESVTLTWELNESAPTDFSDCVKQAWEYSYDL
YKPATVETPYTDEVMKEVMSNFFVESFIGDTPTNYYSGVELHTATCNNVDVAEVGFVGRTL
LNAFNALEYGESKQRTDLVANANAIFDSYLEHGFSPAGFFNEVVHYKRNFIEPVHSIRRQSE
GIYATLYYLNYEKKKGRKHPEWEKRLKGMLDTFLRLQNEDGSFPRKFKDDFSIVDKSGGST
PSATLPLVMGYKYFKDKRYLEAARRSVDYLEKELISKADYFSSTLDANCEDKEASLYASTAA
YYLALASKGAERAHYAGLAKKAAYFALSWYYTWDVPFAPGQMLGDIGLKTRGWGNVSVEN
NHIDVFIFDFADVLRWLSKEYNEPRFADFAAVISSSMRQLLPYEGHQCGIAKKGYYPEVVQH
TNWDYGKNGKGYYNHIFAPGWVVASLWELLSPGRPEEFIGR
SEQ ID NO: 16
MGIEYTPANVSASIALKVPGNEAVRYPLEFRKLRKERFDYQLTATESLPVLIYQKVEGGETN
KRITLFITATENIYFNYGEQVKSGACHDDCLFYMPGFWYRRNLRSPKEAPSFHTSDSWVVR
EDRLSTPMTTIFDEKNGKSFSVARIDKFESDALTTHKEGEVILSGTTSIGYTGFENCNGTATL
SFGYPYKEAPKTYIRKLTLAPSVEAYQFLGKGESVTLTWELNESAPTDFSDCVKQAWEYSY
DLYKPATVETPYTDEVMKEVMSNFFVESFIGDTPTNYYSGVELHTATCNNVDVAEVGFVGR
TLLNAFNALEYGESKQRTDLVANANAIFDSYLEHGFSPAGFFNEVVHYKRNFIEPVHSIRRQ
SEGIYATLYYLNYEKKKGRKHPEWEKRLKGMLDTFLRLQNEDGSFPRKFKDDFSIVDKSGG
STPSATLPLVMGYKYFKDKRYLEAARRSVDYLEKELISKADYFSSTLDANCEDKEASLYAST
AAYYLALASKGAERAHYAGLAKKAAYFALSWYYTWDVPFAPGQMLGDIGLKTRGWGNVSV
ENNHIDVFIFDFADVLRWLSKEYNEPRFADFAAVISSSMRQLLPYEGHQCGIAKKGYYPEVV
QHTNWDYGKNGKGYYNHIFAPGWVVASLWELLSPGRPEEFIGRLEHHHHHH*
SEQ ID NO: 30
MNLRNILSGIALLAGILSAHAIEYTPANVSASIALKVPGNEAVRYPLEFRKLRKERFDYQLTAT
ESLPVLIYQKVEGGETNKRITLFITATENIYFNYGEQVKSGACHDDCLFYMPGFWYRRNLRS
PKEAPSFHTSDSWVVREDRLSTPMTTIFDEKNGKSFSVARIDKFESDALTTHKEGEVILSGT
TSIGYTGFENCNGTATLSFGYPYKEAPKTYIRKLTLAPSVEAYQFLGKGESVTLTWELNESA
PTDFSDCVKQAWEYSYDLYKPATVETPYTDEVMKEVMSNFFVESFIGDTPTNYYSGVELHT
ATCNNVDVAEVGFVGRTLLNAFNALEYGESKQRTDLVANANAIFDSYLEHGFSPAGFFNEV
VHYKRNFIEPVHSIRRQSEGIYATLYYLNYEKKKGRKHPEWEKRLKGMLDTFLRLQNEDGS
FPRKFKDDFSIVDKSGGSTPSATLPLVMGYKYFKDKRYLEAARRSVDYLEKELISKADYFSS
TLDANCEDKEASLYASTAAYYLALASKGAERAHYAGLAKKAAYFALSWYYTWDVPFAPGQ
MLGDIGLKTRGWGNVSVENNHIDVFIFDFADVLRWLSKEYNEPRFADFAAVISSSMRQLLPY
EGHQCGIAKKGYYPEVVQHTNWDYGKNGKGYYNHIFAPGWVVASLWELLSPGRPEEFIGR
SEQ ID NO: 3
QDSEGIYNGASQYRDEIFLADPTILREGDMYYLTGTGNQQQQGFVLLESADLKSWHTVENN
EFVLKKDDQTYGTEGFWAPQWFKAQDRYYFLYTANEQTVMASADSWKGPFRQTDIKPIDR
SQKNIDPFLFRDDDGKYYLYHVRFGNGNFIWVAEFDLAKGEIIPGTLKKCMESTEEWEATAA
YISAPVMEGPTVIKLDGVYYLFYSANDYRSIDYAVGYATSNSPMGPWTKNENSPIIHRSIVGE
NGSGHGDIFTDKAGNYYYVYHVHASKTNVAPRKTRIIPLHMEKNESGIYDITVNGDEVIEPYQE
SEQ ID NO: 17
MQDSEGIYNGASQYRDEIFLADPTILREGDMYYLTGTGNQQQQGFVLLESADLKSWHTVE
NNEFVLKKDDQTYGTEGFWAPQWFKAQDRYYFLYTANEQTVMASADSWKGPFRQTDIKPI
DRSQKNIDPFLFRDDDGKYYLYHVRFGNGNFIWVAEFDLAKGEIIPGTLKKCMESTEEWEA
TAAYISAPVMEGPTVIKLDGVYYLFYSANDYRSIDYAVGYATSNSPMGPWTKNENSPIIHRSI
VGENGSGHGDIFTDKAGNYYYVYHVHASKTNVAPRKTRIIPLHMEKNESGIYDITVNGDEVI
EPYQELEHHHHHH
SEQ ID NO: 31
MRLLHFLIGAMVLIATGCAQDSEGIYNGASQYRDEIFLADPTILREGDMYYLTGTGNQQQQG
FVLLESADLKSWHTVENNEFVLKKDDQTYGTEGFWAPQWFKAQDRYYFLYTANEQTVMA
SADSWKGPFRQTDIKPIDRSQKNIDPFLFRDDDGKYYLYHVRFGNGNFIWVAEFDLAKGEII
PGTLKKCMESTEEWEATAAYISAPVMEGPTVIKLDGVYYLFYSANDYRSIDYAVGYATSNSP
MGPWTKNENSPIIHRSIVGENGSGHGDIFTDKAGNYYYVYHVHASKTNVAPRKTRIIPLHME
KNESGIYDITVNGDEVIEPYQE
SEQ ID NO: 4
HDPEDEDFNVNEDLSVTERRSHSGLDRLKRAIRRRWLQGILHTRAQELVATDNAIARKLHEI
AQDLSNFHLEGPDGNTDFSGHRLKPGESRNLGPVAGTGAPIPGIGSADLGEVIEIPDGKGG
KKLVAVFGDSFSTDHVPRPGEAADHYKSVAVEIKGFDQNGKPIWGDVLTGYDGENGRPPL
FPTTGLPPAAQNTNTLPAGSIVMRDGTTYMMAAGTNDLHPTGGSWLVKAGDPSQGGWPP
EPGTWRAGDKAPSQISGYQAADGTVYIAADSFDRNQQVTMYRVPPGGNVLDRDSWQPMT
NTGWGAPGDPAKPLTTTPYGEVSLREVDGRPVLSGLNMGPDPGNGTARVEVRVGSAGDP
SSVFAWNSPATVLMQQADPTAPNFVLQNYGGYILPGSTLDNMRVFGSQWVVADGTPYNT
QLIEVNPHQ
SEQ ID NO: 18
MGSSHHHHHHSSGLVPRGSHMASMTGGQQMGRGSHDPEDEDFNVNEDLSVTERRSHS
GLDRLKRAIRRRWLQGILHTRAQELVATDNAIARKLHEIAQDLSNFHLEGPDGNTDFSGHRL
KPGESRNLGPVAGTGAPIPGIGSADLGEVIEIPDGKGGKKLVAVFGDSFSTDHVPRPGEAA
DHYKSVAVEIKGFDQNGKPIWGDVLTGYDGENGRPPLFPTTGLPPAAQNTNTLPAGSIVMR
DGTTYMMAAGTNDLHPTGGSWLVKAGDPSQGGWPPEPGTWRAGDKAPSQISGYQAADG
TVYIAADSFDRNQQVTMYRVPPGGNVLDRDSWQPMTNTGWGAPGDPAKPLTTTPYGEVS
LREVDGRPVLSGLNMGPDPGNGTARVEVRVGSAGDPSSVFAWNSPATVLMQQADPTAPN
FVLQNYGGYILPGSTLDNMRVFGSQWVVADGTPYNTQLIEVNPHQ*
SEQ ID NO: 32
MPTKSEILNWRTDHLGAAAKTWGDQATQLDTAVNGAQGLLDGLQGSGQFVEAFRTRLKSY
IDKTRPKVDKLKSAKQVAESSEQQLKGAKDKAVYAVHDPEDEDFNVNEDLSVTERRSHSGL
DRLKRAIRRRWLQGILHTRAQELVATDNAIARKLHEIAQDLSNFHLEGPDGNTDFSGHRLKP
GESRNLGPVAGTGAPIPGIGSADLGEVIEIPDGKGGKKLVAVFGDSFSTDHVPRPGEAADH
YKSVAVEIKGFDQNGKPIWGDVLTGYDGENGRPPLFPTTGLPPAAQNTNTLPAGSIVMRDG
TTYMMAAGTNDLHPTGGSWLVKAGDPSQGGWPPEPGTWRAGDKAPSQISGYQAADGTV
YIAADSFDRNQQVTMYRVPPGGNVLDRDSWQPMTNTGWGAPGDPAKPLTTTPYGEVSLR
EVDGRPVLSGLNMGPDPGNGTARVEVRVGSAGDPSSVFAWNSPATVLMQQADPTAPNFV
LQNYGGYILPGSTLDNMRVFGSQWVVADGTPYNTQLIEVNPHQ
SEQ ID NO: 5
SDDVKEDSGTSKNRKHLKVKKVERVARITGTPVDGENLLSSNNTTTMYDVGGTDLGIVWE
MNQGKYGIFLGDTFGKGFIPDQNDPADFGDNWRCNVLAFSNDCNLEDGLSIDSMASDEND
NARELIPGGKDKSGNGDWTSIPTAAIRANNKDYVHYFNMRNWTGWITNYSGLYKSEDDGK
TWGKCTTVHFTSDSNFGQAGYYKKDGYVYMVGTETGRHSNPRLARFNEKDIENQSLYEY
WNKDHNEWIRGDESQATNLFEDTVGELSIAYHNKYKCWIITYFCAARYNISIRYAEELTGQW
SKPQILATGEEYPQLYGSYIHPVSLDGDDLYFLMSMWRPYNVFLMRAELE
SEQ ID NO: 19
MGSDDVKEDSGTSKNRKHLKVKKVERVARITGTPVDGENLLSSNNTTTMYDVGGTDLGIV
WEMNQGKYGIFLGDTFGKGFIPDQNDPADFGDNWRCNVLAFSNDCNLEDGLSIDSMASDE
NDNARELIPGGKDKSGNGDWTSIPTAAIRANNKDYVHYFNMRNWTGWITNYSGLYKSEDD
GKTWGKCTTVHFTSDSNFGQAGYYKKDGYVYMVGTETGRHSNPRLARFNEKDIENQSLY
EYWNKDHNEWIRGDESQATNLFEDTVGELSIAYHNKYKCWIITYFCAARYNISIRYAEELTG
QWSKPQILATGEEYPQLYGSYIHPVSLDGDDLYFLMSMWRPYNVFLMRAELELEHHHHHH
SEQ ID NO: 33
MKLSRAIKHIFLLGGFLSPLVVMSDDVKEDSGTSKNRKHLKVKKVERVARITGTPVDGENLL
SSNNTTTMYDVGGTDLGIVWEMNQGKYGIFLGDTFGKGFIPDQNDPADFGDNWRCNVLAF
SNDCNLEDGLSIDSMASDENDNARELIPGGKDKSGNGDWTSIPTAAIRANNKDYVHYFNMR
NWTGWITNYSGLYKSEDDGKTWGKCTTVHFTSDSNFGQAGYYKKDGYVYMVGTETGRHS
NPRLARFNEKDIENQSLYEYWNKDHNEWIRGDESQATNLFEDTVGELSIAYHNKYKCWIITY
FCAARYNISIRYAEELTGQWSKPQILATGEEYPQLYGSYIHPVSLDGDDLYFLMSMWRPYN
VFLMRAELE
SEQ ID NO: 6
SKDGEDEPYIPIDEITVSIAELVPDKETKAVSTTLTFKNIGEIDYLLITKSGGESYSAKIEKPELS
ASYPFRYMVKKTDPESFRLLLTAYYKDGNKSKEKVLTIDNRWGFFIRKVSRTARITGNPMSG
EQFPTPNNTAQKWNVGGTDLGIIWEMNPGKYGIFFGDTFGRDFSPNSAAPGPNGGSWRS
NVLAFSDDNDLEDGLTFNSMAVDVSGNAREIIYGGKDASGNGDWTSIPTAAIRANNIDYVHY
FNMKNWTGWITNYSGMYKSIDNGQTWAKCENVRFSSNSNFGQGGYFKKDGYVYMIGTQT
GRDSNARLARFKEQHIEDQANYEFWNSLTKQWLKGDEEQATNIFNDKVGELSFIYNQKHN
KWIISYFNADRYNITMRTATEITGPWSEPYELASGVEYAQLYGSYFHPLSVDGDNLYFLMSM
WLPYNVFLMKAELADMGSFD
SEQ ID NO: 20
MGSKDGEDEPYIPIDEITVSIAELVPDKETKAVSTTLTFKNIGEIDYLLITKSGGESYSAKIEKP
ELSASYPFRYMVKKTDPESFRLLLTAYYKDGNKSKEKVLTIDNRWGFFIRKVSRTARITGNP
MSGEQFPTPNNTAQKWNVGGTDLGIIWEMNPGKYGIFFGDTFGRDFSPNSAAPGPNGGS
WRSNVLAFSDDNDLEDGLTFNSMAVDVSGNAREIIYGGKDASGNGDWTSIPTAAIRANNID
YVHYFNMKNWTGWITNYSGMYKSIDNGQTWAKCENVRFSSNSNFGQGGYFKKDGYVYMI
GTQTGRDSNARLARFKEQHIEDQANYEFWNSLTKQWLKGDEEQATNIFNDKVGELSFIYN
QKHNKWIISYFNADRYNITMRTATEITGPWSEPYELASGVEYAQLYGSYFHPLSVDGDNLYF
LMSMWLPYNVFLMKAELADMGSFDLEHHHHHH
SEQ ID NO: 34
MKTRLFKYIVNIWALAFFTGFYSCSKDGEDEPYIPIDEITVSIAELVPDKETKAVSTTLTFKNIG
EIDYLLITKSGGESYSAKIEKPELSASYPFRYMVKKTDPESFRLLLTAYYKDGNKSKEKVLTID
NRWGFFIRKVSRTARITGNPMSGEQFPTPNNTAQKWNVGGTDLGIIWEMNPGKYGIFFGD
TFGRDFSPNSAAPGPNGGSWRSNVLAFSDDNDLEDGLTFNSMAVDVSGNAREIIYGGKDA
SGNGDWTSIPTAAIRANNIDYVHYFNMKNWTGWITNYSGMYKSIDNGQTWAKCENVRFSS
NSNFGQGGYFKKDGYVYMIGTQTGRDSNARLARFKEQHIEDQANYEFWNSLTKQWLKGD
EEQATNIFNDKVGELSFIYNQKHNKWIISYFNADRYNITMRTATEITGPWSEPYELASGVEYA
QLYGSYFHPLSVDGDNLYFLMSMWLPYNVFLMKAELADMGSFD
SEQ ID NO: 7
APAPRPILPPLAPGQVLRIGPTAGTGTPTGDYGIGATDLCEFVEFPSQLLQVCGDSFAGQGV
GFGGWYAPVALHVDTESIDDPAGVRYTGVTGVGTPLLADPTPPGDSQLPAGVVQINRRNY
LMVTTTKDLQPQNSRLVRAEAARGGWQTVSGSRRNAAYQDGRQTQISGYYDPVPTPDSP
TGWVYIVADSFTRGEPAVLYRATPESFTDRSRWQGWAGGPDGGWNKPPTPLWPDQLGE
MSIRQIDGQTVLSYFNASTGNMEVRVAHHPTSLGAAPVTTVVRHDEWPEPAESLPPPYDN
RLAQPYGGYISPGSTIDELRIFVSQWDTRARQNGPYRVIQFAVNPFKPWSDP
SEQ ID NO: 21
MAPAPRPILPPLAPGQVLRIGPTAGTGTPTGDYGIGATDLCEFVEFPSQLLQVCGDSFAGQ
GVGFGGWYAPVALHVDTESIDDPAGVRYTGVTGVGTPLLADPTPPGDSQLPAGVVQINRR
NYLMVTTTKDLQPQNSRLVRAEAARGGWQTVSGSRRNAAYQDGRQTQISGYYDPVPTPD
SPTGWVYIVADSFTRGEPAVLYRATPESFTDRSRWQGWAGGPDGGWNKPPTPLWPDQL
GEMSIRQIDGQTVLSYFNASTGNMEVRVAHHPTSLGAAPVTTVVRHDEWPEPAESLPPPY
DNRLAQPYGGYISPGSTIDELRIFVSQWDTRARQNGPYRVIQFAVNPFKPWSDPGSGHHH
HHH
SEQ ID NO: 35
MRVCSAWVVRCSLVVSFLVPQCVWYARADPPAPAPRPILPPLAPGQVLRIGPTAGTGTPT
GDYGIGATDLCEFVEFPSQLLQVCGDSFAGQGVGFGGWYAPVALHVDTESIDDPAGVRYT
GVTGVGTPLLADPTPPGDSQLPAGVVQINRRNYLMVTTTKDLQPQNSRLVRAEAARGGWQ
TVSGSRRNAAYQDGRQTQISGYYDPVPTPDSPTGWVYIVADSFTRGEPAVLYRATPESFT
DRSRWQGWAGGPDGGWNKPPTPLWPDQLGEMSIRQIDGQTVLSYFNASTGNMEVRVAH
HPTSLGAAPVTTVVRHDEWPEPAESLPPPYDNRLAQPYGGYISPGSTIDELRIFVSQWDTR
ARQNGPYRVIQFAVNPFKPWSDP
SEQ ID NO: 8
TPSNVTKVSRVTGATPAGELLPNPNQTHTNHDVHGTDLGIVWDKGGGEVFVLFGDTFGAG
WCGNGGCGGGWRSNVLARSSDTNLANGLSFSSMIQDGPRHAKEILASRKVNFDEITVIPTA
GVTIGSRHYIHYMSIHHWGEAGMWFTNHAGIAYSDDQGQNWVKHPTARWQNTPAWTNNF
QMAAFVRNGGFVYMYATPNGRFGNVHLARVPETALLDINGYRYWDGNGWSVSQAAARPV
AIGIAGELSVSYHAPLGRFLMTYLNEHRQAVVMRDAATPTGPWSGEKVLASGAVYPGLYNA
FIHPWGNTGNSLYFVMSQWTPYNTFLMRATLAHD
SEQ ID NO: 22
MGSSHHHHHHSSGLVPRGSHMTPSNVTKVSRVTGATPAGELLPNPNQTHTNHDVHGTDL
GIVWDKGGGEVFVLFGDTFGAGWCGNGGCGGGWRSNVLARSSDTNLANGLSFSSMIQD
GPRHAKEILASRKVNFDEITVIPTAGVTIGSRHYIHYMSIHHWGEAGMWFTNHAGIAYSDDQ
GQNWVKHPTARWQNTPAWTNNFQMAAFVRNGGFVYMYATPNGRFGNVHLARVPETALL
DINGYRYWDGNGWSVSQAAARPVAIGIAGELSVSYHAPLGRFLMTYLNEHRQAVVMRDAA
TPTGPWSGEKVLASGAVYPGLYNAFIHPWGNTGNSLYFVMSQWTPYNTFLMRATLAHD
SEQ ID NO: 36
MKRMTTWKRTFLLHATWALLWGTPAAAVTPSNVTKVSRVTGATPAGELLPNPNQTHTNHD
VHGTDLGIVWDKGGGEVFVLFGDTFGAGWCGNGGCGGGWRSNVLARSSDTNLANGLSF
SSMIQDGPRHAKEILASRKVNFDEITVIPTAGVTIGSRHYIHYMSIHHWGEAGMWFTNHAGIA
YSDDQGQNWVKHPTARWQNTPAWTNNFQMAAFVRNGGFVYMYATPNGRFGNVHLARV
PETALLDINGYRYWDGNGWSVSQAAARPVAIGIAGELSVSYHAPLGRFLMTYLNEHRQAVV
MRDAATPTGPWSGEKVLASGAVYPGLYNAFIHPWGNTGNSLYFVMSQWTPYNTFLMRAT
LAHDAHSDNLLSEPGFETQAATPVMAPWYLQGSGGVDRALGNARSGQDNGFVNASSGW
NALKQSVVVQPYTDYTLRGWVRSSANNTAGYFGARGPGNGPILGEVPFGALGAYTERVVH
FNSGPNALIEVYGGLWADNVDTWMQLDDVSLTRGSNLIHQAGFEQQSTNNATSPWYTEG
RAGIDRGLGYARTGANNAWARNDTGWNAVKQEVAVTPNTQYTLTGWVKTSTNHADGYFG
ARLLRSGPILNEVRFAQPLGNYTQLSVSFNSGAQHSVEVFAGMWANTGDTWIQVDDLTLTRN
SEQ ID NO: 9
LSDRFGMRATDLGAMIEAPNGKLVAVFGDTWKDRVGGIDWRAPVILIGSKGANGVIQWERA
GGSDPNYARQLWAYTHDSPPWTKGGISTVLPSDLLRIGNTLYLHVMVNKGFPTVAWTEIW
KSEDSGISWTHLGENSKLSGTAGNGLLQCLSWDYNADDDYVYAVSTGFQRDKGIVLRRFR
PTQINNQSLWQVWNYNGTWGWRTNVNPSVITPSGEKWGELTLRKLANGWVLGGLSMHSL
GLSYRVLSSPTANLYTTPAKRLIWGCEWPEEDHTVGKVAQTYGGYIVPGSKLGVDGGVDIV
VSQWNTTVGWPYKVMQFRGTL
SEQ ID NO: 23
MGSSHHHHHHSSGLVPRGSHMLSDRFGMRATDLGAMIEAPNGKLVAVFGDTWKDRVGGI
DWRAPVILIGSKGANGVIQWERAGGSDPNYARQLWAYTHDSPPWTKGGISTVLPSDLLRIG
NTLYLHVMVNKGFPTVAWTEIWKSEDSGISWTHLGENSKLSGTAGNGLLQCLSWDYNADD
DYVYAVSTGFQRDKGIVLRRFRPTQINNQSLWQVWNYNGTWGWRTNVNPSVITPSGEKW
GELTLRKLANGWVLGGLSMHSLGLSYRVLSSPTANLYTTPAKRLIWGCEWPEEDHTVGKV
AQTYGGYIVPGSKLGVDGGVDIVVSQWNTTVGWPYKVMQFRGTL
SEQ ID NO: 37
MADRVGIDFSANEIDPAAIKAAGVEAVINYISEARPSAPWMKAVKPMKAEYAKRLRDAGIQIV
SNYQYGKTGDATPSDWRGGYEAGKRHGAIALSNHWKAGGTPWRPCYAPCDDNPTMDEV
TKYVLPFIKGWAEVWGKEYTGIYCNAQTWDWLKSLGAPVTWFWQHNWDGKAEHPHHPD
AHMHQVRIDKDRVGGTGVDWNILLKADYGQWSLSKNPNPIEKFPIVDMIKTSGVGYNAGNS
GRLRVYLHSSEGKDWVSTARGTMQYQATSQTGSYHYLIDDNEIIQTIALNNSAWAVLSDND
VSINICLVLSSGATGYGITARENQPKSRAQWLEHEKMLKMLRFLIDHVCRETGIPKTRVDIKG
VGQNKRGVSSHNNYTYGSVVLKGFKDGTHWDIPDTFPYSYVLEGSVEPTKPIDPNAWPLP
VGYYYGPLDGPEQSISGQFSGDLPEWKDGLGRWQTKVGITASKKWDAQTADAVAKLQKA
KGWFTDSNKMKGLLAKGEWDSVITGGWKITDAKPEAPDPTKPFVPTPKPTGRFMPLVEGT
YKIGSGYGPRDGGFHRGLDFEAKDGTPFYAMQAGTVLYIGPADGYGQWIVVDSDNAQGA
GCVEYGHMWNAFATGLKIGSRVQAGQLLGYVGSNGESTGPHLHVAVHPYEYNPKVYVNP
ADWLRNAKKHVAPSGSDDNKEVPPVTNPEQPASERAVKVSDLTGDGLSDRFGMRATDLG
AMIEAPNGKLVAVFGDTWKDRVGGIDWRAPVILIGSKGANGVIQWERAGGSDPNYARQLW
AYTHDSPPWTKGGISTVLPSDLLRIGNTLYLHVMVNKGFPTVAWTEIWKSEDSGISWTHLG
ENSKLSGTAGNGLLQCLSWDYNADDDYVYAVSTGFQRDKGIVLRRFRPTQINNQSLWQV
WNYNGTWGWRTNVNPSVITPSGEKWGELTLRKLANGWVLGGLSMHSLGLSYRVLSSPTA
NLYTTPAKRLIWGCEWPEEDHTVGKVAQTYGGYIVPGSKLGVDGGVDIVVSQWNTTVGWP
YKVMQFRGTLPAVWPDVVRPDASAVLDAADNLTTAMDTYKGGVAATGPEARQIAVVNAEL
DKLEGIIR
SEQ ID NO: 10
MTPPRRSELWLLDDTRTRSVSAENPTGAPGGGARATTGTGAHAARLLGPGWKISPSIVLG
DGETATLADVSGPGVLRHCWLTTDRAALRELTLRMYWDDAAEPAIDVPLGDFFCNGWDEL
ALVDSEMVVVAPAGGLNSYWPMPFRRRARITLENHCGRSVPVYYQITYGEEDVPGEAGYL
HARWSRSAPLGSPAVHTLLDTGPARGRYVGTYLAIRPGAPRWWGEGEMKFYLDGDDEYP
TLCGTGTEDYFGGAWNFDMDGKYRTYSTAYLGLPQALPADEIYQAHQRFGMYRWHVHDP
ICFHRELRVTVQGLGWRDSDTYLPLEDAEIATTVWWYQEVPPSG
SEQ ID NO: 24
MGSSHHHHHHSSGLVPRGSHMTPPRRSELWLLDDTRTRSVSAENPTGAPGGGARATTGT
GAHAARLLGPGWKISPSIVLGDGETATLADVSGPGVLRHCWLTTDRAALRELTLRMYWDD
AAEPAIDVPLGDFFCNGWDELALVDSEMVVVAPAGGLNSYWPMPFRRRARITLENHCGRS
VPVYYQITYGEEDVPGEAGYLHARWSRSAPLGSPAVHTLLDTGPARGRYVGTYLAIRPGAP
RWWGEGEMKFYLDGDDEYPTLCGTGTEDYFGGAWNFDMDGKYRTYSTAYLGLPQALPA
DEIYQAHQRFGMYRWHVHDPICFHRELRVTVQGLGWRDSDTYLPLEDAEIATTVWWYQEV
PPSG*
SEQ ID NO: 38
MTPPRRSELWLLDDTRTRSVSAENPTGAPGGGARATTGTGAHAARLLGPGWKISPSIVLG
DGETATLADVSGPGVLRHCWLTTDRAALRELTLRMYWDDAAEPAIDVPLGDFFCNGWDEL
ALVDSEMVVVAPAGGLNSYWPMPFRRRARITLENHCGRSVPVYYQITYGEEDVPGEAGYL
HARWSRSAPLGSPAVHTLLDTGPARGRYVGTYLAIRPGAPRWWGEGEMKFYLDGDDEYP
TLCGTGTEDYFGGAWNFDMDGKYRTYSTAYLGLPQALPADEIYQAHQRFGMYRWHVHDP
ICFHRELRVTVQGLGWRDSDTYLPLEDAEIATTVWWYQEVPPSG*
SEQ ID NO: 11
QNPVPGTMYELSQMKNGMRNRRISSNDPAGGVLDHLSDIRPGEKRIIADIPGSGIINHIWITM
APEPHVLNRSDVIIRMYWDGNAYPSVESPIGPFFGQGWNERYNYSALPITAGPANGTSMVS
YFSMPFAQGARIEIENQSDVNLEKFYFYVDYYETKKLPTDLGRFHAWYNQELTEAAPEGET
EWAVIGKQDNNTTGDRNYVFADIKGKGHFVGLNYYVHCPSPIWYGEGDDFWFIDGEEEAS
LLGTGTEDLFNTSWCPKEAYSHPYFGYPRVNNDVGWLGRTHIYRFFIEDPVFFQKSLKASIE
HGHANNLTLDLATVAYWYQSEACPLPPAPSKEVRKLKPFINVPDMHRWRHEWRKNRGED
SKLWGNEMPKQ
SEQ ID NO: 25
MGQNPVPGTMYELSQMKNGMRNRRISSNDPAGGVLDHLSDIRPGEKRIIADIPGSGIINHIW
ITMAPEPHVLNRSDVIIRMYWDGNAYPSVESPIGPFFGQGWNERYNYSALPITAGPANGTS
MVSYFSMPFAQGARIEIENQSDVNLEKFYFYVDYYETKKLPTDLGRFHAWYNQELTEAAPE
GETEWAVIGKQDNNTTGDRNYVFADIKGKGHFVGLNYYVHCPSPIWYGEGDDFWFIDGEE
EASLLGTGTEDLFNTSWCPKEAYSHPYFGYPRVNNDVGWLGRTHIYRFFIEDPVFFQKSLK
ASIEHGHANNLTLDLATVAYWYQSEACPLPPAPSKEVRKLKPFINVPDMHRWRHEWRKNR
GEDSKLWGNEMPKQLEHHHHHH
SEQ ID NO: 39
MKKKPLKLVLICLLLTLFCIPDLVAQNPVPGTMYELSQMKNGMRNRRISSNDPAGGVLDHLS
DIRPGEKRIIADIPGSGIINHIWITMAPEPHVLNRSDVIIRMYWDGNAYPSVESPIGPFFGQGW
NERYNYSALPITAGPANGTSMVSYFSMPFAQGARIEIENQSDVNLEKFYFYVDYYETKKLPT
DLGRFHAWYNQELTEAAPEGETEWAVIGKQDNNTTGDRNYVFADIKGKGHFVGLNYYVHC
PSPIWYGEGDDFWFIDGEEEASLLGTGTEDLFNTSWCPKEAYSHPYFGYPRVNNDVGWLG
RTHIYRFFIEDPVFFQKSLKASIEHGHANNLTLDLATVAYWYQSEACPLPPAPSKEVRKLKPF
INVPDMHRWRHEWRKNRGEDSKLWGNEMPKQ
SEQ ID NO: 12
FSQEGFNGLGVDMSNLHRLSNAKTRSISPENFTGEKGKGGMADPAMQGDKRNVANAHHA
ARDLGQTWKVNPYVHIAPGETFTLAEIEGPGAIQHIWMTPTGNWRFSILRFYWDDEKEPSV
ECPVGDFFGMGWGVYAPLNSLAICVNPGSAFNCYWTMPFRKKCKITMENVNPEDAMVLYY
QVDYTLTDVPADAGYFHAQFRRTHYNETSDYTMIDGVKGKGHYVGTYMAQGVHNNGWW
GEGEIKFFMDGDTKYPTICGTGAEDYFCGSYNFDREGQYKEFCTAYAGLHQVIRPDGTYRS
QQRFGMYRWHIADPVRFEKDLKITIQDLGWRVGGRYLPQKSDISSVVFWYQGEPHAKFPK
FPGWQDLEVN
SEQ ID NO: 26
MGFSQEGFNGLGVDMSNLHRLSNAKTRSISPENFTGEKGKGGMADPAMQGDKRNVANAH
HAARDLGQTWKVNPYVHIAPGETFTLAEIEGPGAIQHIWMTPTGNWRFSILRFYWDDEKEP
SVECPVGDFFGMGWGVYAPLNSLAICVNPGSAFNCYWTMPFRKKCKITMENVNPEDAMVL
YYQVDYTLTDVPADAGYFHAQFRRTHYNETSDYTMIDGVKGKGHYVGTYMAQGVHNNGW
WGEGEIKFFMDGDTKYPTICGTGAEDYFCGSYNFDREGQYKEFCTAYAGLHQVIRPDGTY
RSQQRFGMYRWHIADPVRFEKDLKITIQDLGWRVGGRYLPQKSDISSVVFWYQGEPHAKF
PKFPGWQDLEVNLEHHHHHH
SEQ ID NO: 40
MKKICFLISLFFITQLLFSQEGFNGLGVDMSNLHRLSNAKTRSISPENFTGEKGKGGMADPA
MQGDKRNVANAHHAARDLGQTWKVNPYVHIAPGETFTLAEIEGPGAIQHIWMTPTGNWRF
SILRFYWDDEKEPSVECPVGDFFGMGWGVYAPLNSLAICVNPGSAFNCYWTMPFRKKCKI
TMENVNPEDAMVLYYQVDYTLTDVPADAGYFHAQFRRTHYNETSDYTMIDGVKGKGHYVG
TYMAQGVHNNGWWGEGEIKFFMDGDTKYPTICGTGAEDYFCGSYNFDREGQYKEFCTAY
AGLHQVIRPDGTYRSQQRFGMYRWHIADPVRFEKDLKITIQDLGWRVGGRYLPQKSDISSV
VFWYQGEPHAKFPKFPGWQDLEVN
SEQ ID NO: 13
GEELLEIRKLPKEFQAVIHSIRFDRLGSIKPQQEIKNIDKGLVHVRLIFDLPAEIKQDDWQLTIQ
PAFIPDFNWAPHITPGDMNIIDQHVFRSPAMIVTDNKQGLAVIPDINIMKNGTPTRWYMDMIA
PENKLVLGMSETLVSGHVIYERKEGAVYPKGRVEFGFYLMKYADKEDLENPFRRPLKFFWT
NWGEKLYEEGNPIKGDLEPYVDYTYNWAFNTWKDAVWQEFMLGDKKVGAPVFIVNTTQS
PNYPGKVNEREFLSIWNQAWFSSLRSASGLYRYARRKGNKGLLNKALLSKELALAAPMKE
GFFYGLIGTEMETVETEGKQYNRSKGWDTYYWGNSNRNPYTWDARKSPFHILDMSWTAIL
MLRWYEELEKDERLLEYARKYADALLQKQDAKGFFPGWLDLDTLEPMEYLNDSPETSMSV
TFLLKLYELTKNEKYRSSALRAIDAVCKEIVPVGRWEDFETYWSCCRLGTPEWIGKKIERNN
MYKQCNFSIFWTAEALLDSYRITKNKEYLKLGQRTLDEMLMTQTVWQPPYIYVNAVGGFGV
MNADGEWNDSRQSLFAETILQYGKLLNKKEYNERGLSAIRASFSMMYCPENPLAKEQWER
VWPFFNEKDYGFTMENYGHDGRTSKDGIGIGEFTIYDWGNGAAAEAYNRIRDKFGIEIFR
SEQ ID NO: 27
MGGEELLEIRKLPKEFQAVIHSIRFDRLGSIKPQQEIKNIDKGLVHVRLIFDLPAEIKQDDWQL
TIQPAFIPDFNWAPHITPGDMNIIDQHVFRSPAMIVTDNKQGLAVIPDINIMKNGTPTRWYMD
MIAPENKLVLGMSETLVSGHVIYERKEGAVYPKGRVEFGFYLMKYADKEDLENPFRRPLKF
FWTNWGEKLYEEGNPIKGDLEPYVDYTYNWAFNTWKDAVWQEFMLGDKKVGAPVFIVNT
TQSPNYPGKVNEREFLSIWNQAWFSSLRSASGLYRYARRKGNKGLLNKALLSKELALAAP
MKEGFFYGLIGTEMETVETEGKQYNRSKGWDTYYWGNSNRNPYTWDARKSPFHILDMSW
TAILMLRWYEELEKDERLLEYARKYADALLQKQDAKGFFPGWLDLDTLEPMEYLNDSPETS
MSVTFLLKLYELTKNEKYRSSALRAIDAVCKEIVPVGRWEDFETYWSCCRLGTPEWIGKKIE
RNNMYKQCNFSIFWTAEALLDSYRITKNKEYLKLGQRTLDEMLMTQTVWQPPYIYVNAVGG
FGVMNADGEWNDSRQSLFAETILQYGKLLNKKEYNERGLSAIRASFSMMYCPENPLAKEQ
WERVWPFFNEKDYGFTMENYGHDGRTSKDGIGIGEFTIYDWGNGAAAEAYNRIRDKFGIEI
FRLEHHHHHH
SEQ ID NO: 41
MLVISFLSLFSSLLFPVRVLGEELLEIRKLPKEFQAVIHSIRFDRLGSIKPQQEIKNIDKGLVHV
RLIFDLPAEIKQDDWQLTIQPAFIPDFNWAPHITPGDMNIIDQHVFRSPAMIVTDNKQGLAVIP
DINIMKNGTPTRWYMDMIAPENKLVLGMSETLVSGHVIYERKEGAVYPKGRVEFGFYLMKY
ADKEDLENPFRRPLKFFWTNWGEKLYEEGNPIKGDLEPYVDYTYNWAFNTWKDAVWQEF
MLGDKKVGAPVFIVNTTQSPNYPGKVNEREFLSIWNQAWFSSLRSASGLYRYARRKGNKG
LLNKALLSKELALAAPMKEGFFYGLIGTEMETVETEGKQYNRSKGWDTYYWGNSNRNPYT
WDARKSPFHILDMSWTAILMLRWYEELEKDERLLEYARKYADALLQKQDAKGFFPGWLDL
DTLEPMEYLNDSPETSMSVTFLLKLYELTKNEKYRSSALRAIDAVCKEIVPVGRWEDFETYW
SCCRLGTPEWIGKKIERNNMYKQCNFSIFWTAEALLDSYRITKNKEYLKLGQRTLDEMLMT
QTVWQPPYIYVNAVGGFGVMNADGEWNDSRQSLFAETILQYGKLLNKKEYNERGLSAIRA
SFSMMYCPENPLAKEQWERVWPFFNEKDYGFTMENYGHDGRTSKDGIGIGEFTIYDWGN
GAAAEAYNRIRDKFGIEIFR
SEQ ID NO: 14
ARLPQPGGDMGNWGNILNDYLRVAHDEQGNIKAGAIGATQVVDGALPQAKIQNLTTDLSAK
YTKPGAGIPKTDLSASVQASLDKADTALQSVPPSAGVATLSGFPLRLAGEKQVSYPIELGKA
HTPVANPTGAKKVLLAESWGTAGVLKHIWVASSGGGGVGDFSENGGKIRIYIDDDTTAVVD
LSLNDFFAYASQGGEYATRRIGRTKRNTSNGESSAYRYLHMPFQKYLRVEVENTTATDIVF
FGSADYTLVNDFAGLGSQQLAYKMYSVEAAAASPYDELTVVDINGSGQVESLWIAVNAASG
DTGILEGNVEVYVDGETYPSWRSSGTEDAFNGGWYNVPVGGYPAGRAGDSAQSGLAMTY
YRFFVDDPIFFSSHLKVLIHAGQPNQGTITSSTFGLSGFAGVWSNTAGNVNYLAVDTSTTLL
NDQFTDTAGSLDTAKWNQVGGVTQGQATGSTITVAYDGSSSGQDVRIARKNAVLPTNYWV
ETRVRITDATHDGQEVSLIAQGNSPDPYFGSAIHVQLVRFGQNNWVIRARDDFDEVFIRTIG
GGRNLTNVWVKLAFKVVGATVTAYWAPDSSPVWQPVGTWVTGKTGSAFGIGTWTAGAEF
DYLVVRPLQTYTH
SEQ ID NO: 28
MGARLPQPGGDMGNWGNILNDYLRVAHDEQGNIKAGAIGATQVVDGALPQAKIQNLTTDL
SAKYTKPGAGIPKTDLSASVQASLDKADTALQSVPPSAGVATLSGFPLRLAGEKQVSYPIEL
GKAHTPVANPTGAKKVLLAESWGTAGVLKHIWVASSGGGGVGDFSENGGKIRIYIDDDTTA
VVDLSLNDFFAYASQGGEYATRRIGRTKRNTSNGESSAYRYLHMPFQKYLRVEVENTTATD
IVFFGSADYTLVNDFAGLGSQQLAYKMYSVEAAAASPYDELTVVDINGSGQVESLWIAVNA
ASGDTGILEGNVEVYVDGETYPSWRSSGTEDAFNGGWYNVPVGGYPAGRAGDSAQSGLA
MTYYRFFVDDPIFFSSHLKVLIHAGQPNQGTITSSTFGLSGFAGVWSNTAGNVNYLAVDTST
TLLNDQFTDTAGSLDTAKWNQVGGVTQGQATGSTITVAYDGSSSGQDVRIARKNAVLPTN
YWVETRVRITDATHDGQEVSLIAQGNSPDPYFGSAIHVQLVRFGQNNWVIRARDDFDEVFI
RTIGGGRNLTNVWVKLAFKVVGATVTAYWAPDSSPVWQPVGTWVTGKTGSAFGIGTWTA
GAEFDYLVVRPLQTYTHLEHHHHHH
SEQ ID NO: 42
MARLPQPGGDMGNWGNILNDYLRVAHDEQGNIKAGAIGATQVVDGALPQAKIQNLTTDLS
AKYTKPGAGIPKTDLSASVQASLDKADTALQSVPPSAGVATLSGFPLRLAGEKQVSYPIELG
KAHTPVANPTGAKKVLLAESWGTAGVLKHIWVASSGGGGVGDFSENGGKIRIYIDDDTTAV
VDLSLNDFFAYASQGGEYATRRIGRTKRNTSNGESSAYRYLHMPFQKYLRVEVENTTATDI
VFFGSADYTLVNDFAGLGSQQLAYKMYSVEAAAASPYDELTVVDINGSGQVESLWIAVNAA
SGDTGILEGNVEVYVDGETYPSWRSSGTEDAFNGGWYNVPVGGYPAGRAGDSAQSGLA
MTYYRFFVDDPIFFSSHLKVLIHAGQPNQGTITSSTFGLSGFAGVWSNTAGNVNYLAVDTST
TLLNDQFTDTAGSLDTAKWNQVGGVTQGQATGSTITVAYDGSSSGQDVRIARKNAVLPTN
YWVETRVRITDATHDGQEVSLIAQGNSPDPYFGSAIHVQLVRFGQNNWVIRARDDFDEVFI
RTIGGGRNLTNVWVKLAFKVVGATVTAYWAPDSSPVWQPVGTWVTGKTGSAFGIGTWTA
GAEFDYLVVRPLQTYTH

1. Use of an enzyme for cleaving a linkage between two D-galactofuranoses in a polysaccharide, wherein the enzyme comprises an amino acid sequence having at least 70% sequence identity to a sequence provided in Table 1.

2. Use according to claim 1, wherein the enzyme is an exo-D-galactofuranosidase, and the enzyme comprises an amino acid sequence as shown in SEQ ID NO: 1 or SEQ ID NO: 2; or an amino acid sequence which has at least 70% sequence identity thereto.

3. Use according to claim 2, wherein the exo-D-galactofuranosidase comprises an amino acid sequence as shown in SEQ ID NO: 1 or SEQ ID NO: 2.

4. Use according to claim 1, wherein the enzyme is an endo-D-galactofuranase, and the enzyme comprises an amino acid sequence as shown in SEQ ID NO: 3; or an amino acid sequence which has at least 70% sequence identity thereto.

5. Use according to claim 4, wherein the endo-D-galactofuranase comprises an amino acid sequence as shown in SEQ ID NO: 3.

6. Use according to any one of claims 1 to 5, wherein the polysaccharide is a D-galactofuranose polysaccharide.

7. Use of an enzyme for cleaving a linkage between two D-arabinofuranoses in a polysaccharide, wherein the enzyme comprises an amino acid sequence having at least 70% sequence identity to a sequence provided in Table 2.

8. Use according to claim 7, wherein the enzyme is an endo-D-arabinofuranase, and the enzyme comprises an amino acid sequence as shown in SEQ ID NO: 4, SEQ ID NO: 5, SEQ ID NO: 6, SEQ ID NO: 7, SEQ ID NO: 8, or SEQ ID NO: 9; or an amino acid sequence which has at least 70% sequence identity thereto.

9. Use according to claim 8, wherein the endo-D-arabinofuranase comprises an amino acid sequence as shown in SEQ ID NO: 4, SEQ ID NO: 5, SEQ ID NO: 6, SEQ ID NO: 7, SEQ ID NO: 8, or SEQ ID NO: 9.

10. Use according to claim 7, wherein the enzyme is an exo-D-arabinofuranosidase, and the enzyme comprises an amino acid sequence as shown in SEQ ID NO: 10, SEQ ID NO: 11, SEQ ID NO: 12, SEQ ID NO: 13 or SEQ ID NO: 14; or an amino acid sequence which has at least 70% sequence identity thereto.

11. Use according to claim 10, wherein the exo-D-arabinofuranosidase comprises an amino acid sequence as shown in SEQ ID NO: 10, SEQ ID NO: 11, SEQ ID NO: 12, SEQ ID NO: 13 or SEQ ID NO: 14.

12. Use according to any one of claims 7 to 11, wherein the polysaccharide is lipoarabinomannan (LAM) or pilin oligosaccharide.

13. Use according to any one of claims 1 to 5, or 7 to 11 wherein the polysaccharide is arabinogalactan.

14. Use according to any proceeding claim, wherein the polysaccharide is a bacterial cell wall polysaccharide.

15. Use according to claim 14, wherein the bacterial cell wall polysaccharide is from a bacterium of the Actinomycetota phylum.

16. Use according to claim 15, wherein the bacterium is of the Mycobacteriales order.

17. Use according to claim 16, wherein the bacterium is of the family selected from the group consisting of: Segniliparaceae, Mycobacteriaceae, Nocardiaceae, Tsukamurellaceae, Gordoniaceae, Lawsonellaceae, Corynebacteriaceae and Dietziaceae.

18. Use according to claim 17, wherein the bacterium is of the family Mycobacteriaceae.

19. Use according to claim 18, wherein the bacterium is selected from the group consisting of M. tuberculosis, M. bovis, M. africanum, M. canetti, M. microti. M. smegmatis, M. fortuitum, M. marinum, M. ulcerans, M. paratuberculosis, M. celatum, M. avium, M. leprae, M. lepraemurium, M. intracellulare M. scrofulaceum, M. xenopi, M. genavense, M. kansasii, M. simiae, M. szulgai, M. haemophilum, M. asiaticum, M. malmoense, M. vaccae, M. caprae, M. pinnipe dii and M. shimoidei.

20. A composition for degrading a Actinomycetota bacterial cell wall polysaccharide, wherein the composition comprises at least two bacterial cell wall degrading enzymes, wherein at least one of the at least two cell wall degrading enzymes is an enzyme comprising an amino acid sequence as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10; or an amino acid sequence which has at least 70% sequence identity thereto.

21. The composition according to claim 20, wherein the composition comprises:
a) at least one enzyme comprising an amino acid sequence as shown in SEQ ID NO: 1, SEQ ID NO: 2, or SEQ ID NO: 3; or an amino acid sequence which has at least 70% sequence identity thereto; and
b) at least one enzyme comprising an amino acid sequence as shown in SEQ ID NO: 4, SEQ ID NO: 5, or SEQ ID NO: 6, SEQ ID NO: 7, SEQ ID NO: 8, SEQ ID NO: 9, or SEQ ID NO: 10;
or an amino acid sequence which has at least 70% sequence identity thereto.

22. The composition according to claim 20 or 21, wherein the composition comprises an exo-D-galactofuranosidase comprising an amino acid sequence as shown in SEQ ID NO: 1 or SEQ ID NO: 2; or an amino acid sequence which has at least 70% sequence identity thereto.

23. The composition according to any one of claims 20 to 22, wherein the composition comprises an endo-D-galactofuranase comprising an amino acid sequence as shown in SEQ ID NO: 3; or an amino acid sequence which has at least 70% sequence identity thereto.

24. The composition according to any one of claims 20 to 23, wherein the composition comprises an endo-D-arabinofuranase comprising an amino acid sequence as shown in SEQ ID NO: 4, SEQ ID NO: 5 or SEQ ID NO: 6; or an amino acid sequence which has at least 70% sequence identity thereto.

25. The composition according to any one of claims 20 to 24, wherein the composition comprises an exo-D-arabinofuranosidase comprising an amino acid sequence as shown in SEQ ID NO: 7, SEQ ID NO: 8, SEQ ID NO: 9, or SEQ ID NO: 10; or an amino acid sequence which has at least 70% sequence identity thereto.

26. The composition according to any one of claims 20 to 25, wherein the composition comprises:
(i) an exo-D-galactofuranosidase comprising an amino acid sequence as shown in SEQ ID NO: 1 or SEQ ID NO: 2; or an amino acid sequence which has at least 70% sequence identity thereto;
(ii) an endo-D-galactofuranase comprising an amino acid sequence as shown in SEQ ID NO: 3; or an amino acid sequence which has at least 70% sequence identity thereto; and
(iii) an endo-D-arabinofuranase comprising an amino acid sequence as shown in SEQ ID NO: 4, SEQ ID NO: 5 or SEQ ID NO: 6; or an amino acid sequence which has at least 70% sequence identity thereto.

27. The composition according to claim 26, further comprising an exo-D-arabinofuranosidase comprising an amino acid sequence as shown in SEQ ID NO: 7, SEQ ID NO: 8, SEQ ID NO: 9, or SEQ ID NO: 10; or an amino acid sequence which has at least 70% sequence identity thereto.

28. The composition according to any one of claims 20 to 27, wherein the composition comprises:
(i) an exo-D-galactofuranosidase comprising an amino acid sequence as shown in SEQ ID NO: 1 and/or SEQ ID NO: 2; or an amino acid sequence which has at least 70% sequence identity thereto;
(ii) an endo-D-galactofuranase comprising an amino acid sequence as shown in SEQ ID NO: 3; or an amino acid sequence which has at least 70% sequence identity thereto;
(iii) an endo-D-arabinofuranase comprising an amino acid sequence as shown in SEQ ID NO: 5 and/or SEQ ID NO: 6; or an amino acid sequence which has at least 70% sequence identity thereto; and
(iv) an exo-D-arabinofuranosidase comprising an amino acid sequence as shown in SEQ ID NO: 8 and/or SEQ ID NO: 10; or an amino acid sequence which has at least 70% sequence identity thereto.

29. The composition according to claim 28, wherein the composition comprises:
(i) an exo-D-galactofuranosidase comprising an amino acid sequence as shown in SEQ ID NO: 1 and/or SEQ ID NO: 2;
(ii) an endo-D-galactofuranase comprising an amino acid sequence as shown in SEQ ID NO: 3;
(iii) an endo-D-arabinofuranase comprising an amino acid sequence as shown in SEQ ID NO: 5 and/or SEQ ID NO: 6; and
(iv) an exo-D-arabinofuranosidase comprising an amino acid sequence as shown in SEQ ID NO: 8 and/or SEQ ID NO: 10.

30. The composition according to claim 29, wherein the composition comprises:
(i) an exo-D-galactofuranosidase comprising an amino acid sequence as shown in SEQ ID NO: 1;
(ii) an endo-D-galactofuranase comprising an amino acid sequence as shown in SEQ ID NO: 3;
(iii) an endo-D-arabinofuranase comprising an amino acid sequence as shown in SEQ ID NO: 5 and SEQ ID NO: 6; and
(iv) an exo-D-arabinofuranosidase comprising an amino acid sequence as shown in SEQ ID NO: 8 and SEQ ID NO: 10.

31. The composition according to any one of claims 20 to 26, wherein the composition comprises an exo-D-arabinofuranosidase comprising an amino acid sequence as shown in SEQ ID NO: 11 and/or SEQ ID NO: 13; or an amino acid sequence which has at least 70% sequence identity thereto.

32. The composition according to claim 31, wherein the composition comprises:
(v) an exo-D-galactofuranosidase comprising an amino acid sequence as shown in SEQ ID NO: 1; or an amino acid sequence which has at least 70% sequence identity thereto;
(vi) an endo-D-galactofuranase comprising an amino acid sequence as shown in SEQ ID NO: 3; or an amino acid sequence which has at least 70% sequence identity thereto;
(vii) an endo-D-arabinofuranase comprising an amino acid sequence as shown in SEQ ID NO: 6; or an amino acid sequence which has at least 70% sequence identity thereto; and
(viii) an exo-D-arabinofuranosidase comprising an amino acid sequence as shown in SEQ ID NO: 11 and/or SEQ ID NO: 13; or an amino acid sequence which has at least 70% sequence identity thereto.

33. The composition according to claim 32, wherein the composition comprises:
(v) an exo-D-galactofuranosidase comprising an amino acid sequence as shown in SEQ ID NO: 1;
(vi) an endo-D-galactofuranase comprising an amino acid sequence as shown in SEQ ID NO: 3;
(vii) an endo-D-arabinofuranase comprising an amino acid sequence as shown in SEQ ID NO: 6; and
(viii) an exo-D-arabinofuranosidase comprising an amino acid sequence as shown in SEQ ID NO: 11 and SEQ ID NO: 13.

34. The composition according to any one of claims 20 to 33, wherein the composition is freeze dried.

35. The composition according to any one of claims 20 to 34, wherein the second of the at least two cell wall degrading enzymes is selected from the group consisting of a peptidoglycan degrading enzyme, a mycolic acid degrading enzyme, a lipoarabinomannan degrading enzyme, and a pilin oligosaccharide degrading enzyme.

36. The composition according to claim 35, wherein the mycolic acid degrading enzyme is lysin B or a Candida rugosa lipase.

37. The composition according to claim 35 or 36, wherein the peptidoglycan degrading enzyme is a lysozyme, optionally hen-egg white lysozyme.

38. Use of a composition according to any one of claims 21 to 37, for cleaving a linkage between two D-galactofuranoses and/or cleaving a linkage between two D-arabinofuranoses in a polysaccharide.

39. A method for preparing a sample for the detection of a bacterium of the Actinomycetota phylum, the method comprising contacting the sample with a composition according to any one of claims 21 to 37 under conditions that allow for degradation of Actinomycetota bacterial cell wall polysaccharides to occur.

40. The method according to claim 39, wherein the method lyses the bacteria in the sample.

41. A method of determining the presence of a bacterium of the Actinomycetota phylum in a sample, comprising:
(i) providing a sample prepared according to claim 39 or 40; and
(ii) detecting a biomarker indicative of the presence of the bacterium in the sample.

42. The method of claim 41, wherein the biomarker is a nucleic acid, protein, carbohydrate, and/or a lipid.

43. The method according to any one of claims 39 to 42, wherein the sample is selected from the group consisting of a sputum sample, a blood sample, a stool sample and a urine sample.

44. The method according to any one of claims 39 to 43, wherein the bacterium is of the Mycobacteriales order.

45. The method according to claim 44, wherein the bacterium is of the family selected from the group consisting of: Segniliparaceae, Mycobacteriaceae, Nocardiaceae, Tsukamurellaceae, Gordoniaceae, Lawsonellaceae, Corynebacteriaceae and Dietziaceae.

46. The method according to claim 45, wherein the bacterium is of the family Mycobacteriaceae, optionally wherein the bacterium is selected from the group consisting of M. tuberculosis, M. bovis, M. africanum, M. canetti, M. microti. M. smegmatis, M. fortuitum, M. marinum, M. ulcerans, M. paratuberculosis, M. celatum, M. avium, M. leprae, M. lepraemurium, M. intracellulare M. scrofulaceum, M. xenopi, M. genavense, M. kansasii, M. simiae, M. szulgai, M. haemophilum, M. asiaticum, M. malmoense, M. vaccae, M. caprae, M. pinnipe dii and M. shimoidei.

47. A pharmaceutical formulation comprising a composition according to any one of claims 20 to 37, and a pharmaceutically acceptable excipient carrier, adjuvant, and/or diluent.

48. The pharmaceutical formulation according to claim 47, wherein the formulation is for pulmonary delivery and/or topical administration.

49. A pharmaceutical formulation according to claim 47 or 48 for use as a medicament.

50. A pharmaceutical formulation according to claim 47 or 48 for use in treating a bacterial infection caused by a bacterium of the Actinomycetota phylum.

51. The pharmaceutical formulation for use according to claim 50, wherein the infection is a mycobacterial infection.

52. A method of treating a bacterial infection caused by bacterium of the Actinomycetota phylum in a subject, comprising administering a therapeutically effective amount of the pharmaceutical formulation according to claim 47 or 48 to a subject in need thereof.

53. The method according to claim 52, wherein the infection is a mycobacterial infection.


SEQUENCE LISTING (48 sequences)

SEQ ID NO: 1 - protein, 656 aa, synthetic construct
  REGION 1..656: note = Exo-D-galactofuranosidase: BACFIN_04787 tested enzyme
MVNYVPENISASISLKVPGNVAERYSLNLQQLGNNRSSYLLEASESIPVTITQRVADVNGKLRVNVCITALEDIYFNYSQRLSTGYRHDDCQFYMPGFWYRRNLRSPKDAPSFHSSDSWLVREDRLSAPLTGVYSEKEKHFVTVSRIDKFAEEALSTHREGEIILSGKTSLGFTGFENQDGITALSFGFPYREAPKSYIRKLTLAPAVEAFQFLKKGETVLLTWEIQENKADDYSDFIRHAWEYSYDTYGPKPVETPYSITDMKQVLSGFFISSFVEGHPLVYNSGIHLMTENCQSNGQAEVGFIGRVLLNAFNAWEYGHQSDREDLKANSMKVFDSYLKNGFTPVGFFKESVDFDKGYEDPVHSIRRQSEGIYAMLHFLAYEKENGRRHPEWEQKMKNMLDILLRLQQADGSFPRKFRDDFTIVDTSGGSTPSATLPLVMGYKYFKDKRYLASAKQTADYLEKVLISKADYFSSTLDANCEDKEASLYAATATYYLSLITKGDEHRHYADLTKKAAYFALSWYYVWDVPFAQGQMLGDIGLKTRGWGNVSVENNHIDVFVFEFASVLRWLSKEYAEPRFSQFAEVISTSMRQLLPHEGHLCGVAKPGYYPEVVQHTNWDYGKNGKGYYNDIFAPGWTVASLWELFTPGRAEYFLK

SEQ ID NO: 2 - protein, 656 aa, Bacteroides finegoldii
IEYTPANVSASIALKVPGNEAVRYPLEFRKLRKERFDYQLTATESLPVLIYQKVEGGETNKRITLFITATENIYFNYGEQVKSGACHDDCLFYMPGFWYRRNLRSPKEAPSFHTSDSWVVREDRLSTPMTTIFDEKNGKSFSVARIDKFESDALTTHKEGEVILSGTTSIGYTGFENCNGTATLSFGYPYKEAPKTYIRKLTLAPSVEAYQFLGKGESVTLTWELNESAPTDFSDCVKQAWEYSYDLYKPATVETPYTDEVMKEVMSNFFVESFIGDTPTNYYSGVELHTATCNNVDVAEVGFVGRTLLNAFNALEYGESKQRTDLVANANAIFDSYLEHGFSPAGFFNEVVHYKRNFIEPVHSIRRQSEGIYATLYYLNYEKKKGRKHPEWEKRLKGMLDTFLRLQNEDGSFPRKFKDDFSIVDKSGGSTPSATLPLVMGYKYFKDKRYLEAARRSVDYLEKELISKADYFSSTLDANCEDKEASLYASTAAYYLALASKGAERAHYAGLAKKAAYFALSWYYTWDVPFAPGQMLGDIGLKTRGWGNVSVENNHIDVFIFDFADVLRWLSKEYNEPRFADFAAVISSSMRQLLPYEGHQCGIAKKGYYPEVVQHTNWDYGKNGKGYYNHIFAPGWVVASLWELLSPGRPEEFIGR

SEQ ID NO: 3 - protein, 310 aa, Bacteroides finegoldii
QDSEGIYNGASQYRDEIFLADPTILREGDMYYLTGTGNQQQQGFVLLESADLKSWHTVENNEFVLKKDDQTYGTEGFWAPQWFKAQDRYYFLYTANEQTVMASADSWKGPFRQTDIKPIDRSQKNIDPFLFRDDDGKYYLYHVRFGNGNFIWVAEFDLAKGEIIPGTLKKCMESTEEWEATAAYISAPVMEGPTVIKLDGVYYLFYSANDYRSIDYAVGYATSNSPMGPWTKNENSPIIHRSIVGENGSGHGDIFTDKAGNYYYVYHVHASKTNVAPRKTRIIPLHMEKNESGIYDITVNGDEVIEPYQE

SEQ ID NO: 4 - protein, 428 aa, Mycobacteroides abscessus
HDPEDEDFNVNEDLSVTERRSHSGLDRLKRAIRRRWLQGILHTRAQELVATDNAIARKLHEIAQDLSNFHLEGPDGNTDFSGHRLKPGESRNLGPVAGTGAPIPGIGSADLGEVIEIPDGKGGKKLVAVFGDSFSTDHVPRPGEAADHYKSVAVEIKGFDQNGKPIWGDVLTGYDGENGRPPLFPTTGLPPAAQNTNTLPAGSIVMRDGTTYMMAAGTNDLHPTGGSWLVKAGDPSQGGWPPEPGTWRAGDKAPSQISGYQAADGTVYIAADSFDRNQQVTMYRVPPGGNVLDRDSWQPMTNTGWGAPGDPAKPLTTTPYGEVSLREVDGRPVLSGLNMGPDPGNGTARVEVRVGSAGDPSSVFAWNSPATVLMQQADPTAPNFVLQNYGGYILPGSTLDNMRVFGSQWVVADGTPYNTQLIEVNPHQ

SEQ ID NO: 5 - protein, 351 aa, Dysgonomonas gadei
SDDVKEDSGTSKNRKHLKVKKVERVARITGTPVDGENLLSSNNTTTMYDVGGTDLGIVWEMNQGKYGIFLGDTFGKGFIPDQNDPADFGDNWRCNVLAFSNDCNLEDGLSIDSMASDENDNARELIPGGKDKSGNGDWTSIPTAAIRANNKDYVHYFNMRNWTGWITNYSGLYKSEDDGKTWGKCTTVHFTSDSNFGQAGYYKKDGYVYMVGTETGRHSNPRLARFNEKDIENQSLYEYWNKDHNEWIRGDESQATNLFEDTVGELSIAYHNKYKCWIITYFCAARYNISIRYAEELTGQWSKPQILATGEEYPQLYGSYIHPVSLDGDDLYFLMSMWRPYNVFLMRAELE

SEQ ID NO: 6 - protein, 450 aa, Dysgonomonas gadei
SKDGEDEPYIPIDEITVSIAELVPDKETKAVSTTLTFKNIGEIDYLLITKSGGESYSAKIEKPELSASYPFRYMVKKTDPESFRLLLTAYYKDGNKSKEKVLTIDNRWGFFIRKVSRTARITGNPMSGEQFPTPNNTAQKWNVGGTDLGIIWEMNPGKYGIFFGDTFGRDFSPNSAAPGPNGGSWRSNVLAFSDDNDLEDGLTFNSMAVDVSGNAREIIYGGKDASGNGDWTSIPTAAIRANNIDYVHYFNMKNWTGWITNYSGMYKSIDNGQTWAKCENVRFSSNSNFGQGGYFKKDGYVYMIGTQTGRDSNARLARFKEQHIEDQANYEFWNSLTKQWLKGDEEQATNIFNDKVGELSFIYNQKHNKWIISYFNADRYNITMRTATEITGPWSEPYELASGVEYAQLYGSYFHPLSVDGDNLYFLMSMWLPYNVFLMKAELADMGSFD

SEQ ID NO: 7 - protein, 351 aa, Mycobacterium tuberculosis
APAPRPILPPLAPGQVLRIGPTAGTGTPTGDYGIGATDLCEFVEFPSQLLQVCGDSFAGQGVGFGGWYAPVALHVDTESIDDPAGVRYTGVTGVGTPLLADPTPPGDSQLPAGVVQINRRNYLMVTTTKDLQPQNSRLVRAEAARGGWQTVSGSRRNAAYQDGRQTQISGYYDPVPTPDSPTGWVYIVADSFTRGEPAVLYRATPESFTDRSRWQGWAGGPDGGWNKPPTPLWPDQLGEMSIRQIDGQTVLSYFNASTGNMEVRVAHHPTSLGAAPVTTVVRHDEWPEPAESLPPPYDNRLAQPYGGYISPGSTIDELRIFVSQWDTRARQNGPYRVIQFAVNPFKPWSDP

SEQ ID NO: 8 - protein, 334 aa, Myxococcus xanthus
TPSNVTKVSRVTGATPAGELLPNPNQTHTNHDVHGTDLGIVWDKGGGEVFVLFGDTFGAGWCGNGGCGGGWRSNVLARSSDTNLANGLSFSSMIQDGPRHAKEILASRKVNFDEITVIPTAGVTIGSRHYIHYMSIHHWGEAGMWFTNHAGIAYSDDQGQNWVKHPTARWQNTPAWTNNFQMAAFVRNGGFVYMYATPNGRFGNVHLARVPETALLDINGYRYWDGNGWSVSQAAARPVAIGIAGELSVSYHAPLGRFLMTYLNEHRQAVVMRDAATPTGPWSGEKVLASGAVYPGLYNAFIHPWGNTGNSLYFVMSQWTPYNTFLMRATLAHD

SEQ ID NO: 9 - protein, 322 aa, Gordonia Phage
LSDRFGMRATDLGAMIEAPNGKLVAVFGDTWKDRVGGIDWRAPVILIGSKGANGVIQWERAGGSDPNYARQLWAYTHDSPPWTKGGISTVLPSDLLRIGNTLYLHVMVNKGFPTVAWTEIWKSEDSGISWTHLGENSKLSGTAGNGLLQCLSWDYNADDDYVYAVSTGFQRDKGIVLRRFRPTQINNQSLWQVWNYNGTWGWRTNVNPSVITPSGEKWGELTLRKLANGWVLGGLSMHSLGLSYRVLSSPTANLYTTPAKRLIWGCEWPEEDHTVGKVAQTYGGYIVPGSKLGVDGGVDIVVSQWNTTVGWPYKVMQFRGTL

SEQ ID NO: 10 - protein, 342 aa, Nocardia brasiliensis
MTPPRRSELWLLDDTRTRSVSAENPTGAPGGGARATTGTGAHAARLLGPGWKISPSIVLGDGETATLADVSGPGVLRHCWLTTDRAALRELTLRMYWDDAAEPAIDVPLGDFFCNGWDELALVDSEMVVVAPAGGLNSYWPMPFRRRARITLENHCGRSVPVYYQITYGEEDVPGEAGYLHARWSRSAPLGSPAVHTLLDTGPARGRYVGTYLAIRPGAPRWWGEGEMKFYLDGDDEYPTLCGTGTEDYFGGAWNFDMDGKYRTYSTAYLGLPQALPADEIYQAHQRFGMYRWHVHDPICFHRELRVTVQGLGWRDSDTYLPLEDAEIATTVWWYQEVPPSG

SEQ ID NO: 11 - protein, 377 aa, Dysgonomonas gadei
QNPVPGTMYELSQMKNGMRNRRISSNDPAGGVLDHLSDIRPGEKRIIADIPGSGIINHIWITMAPEPHVLNRSDVIIRMYWDGNAYPSVESPIGPFFGQGWNERYNYSALPITAGPANGTSMVSYFSMPFAQGARIEIENQSDVNLEKFYFYVDYYETKKLPTDLGRFHAWYNQELTEAAPEGETEWAVIGKQDNNTTGDRNYVFADIKGKGHFVGLNYYVHCPSPIWYGEGDDFWFIDGEEEASLLGTGTEDLFNTSWCPKEAYSHPYFGYPRVNNDVGWLGRTHIYRFFIEDPVFFQKSLKASIEHGHANNLTLDLATVAYWYQSEACPLPPAPSKEVRKLKPFINVPDMHRWRHEWRKNRGEDSKLWGNEMPKQ

SEQ ID NO: 12 - protein, 369 aa, Dysgonomonas gadei
FSQEGFNGLGVDMSNLHRLSNAKTRSISPENFTGEKGKGGMADPAMQGDKRNVANAHHAARDLGQTWKVNPYVHIAPGETFTLAEIEGPGAIQHIWMTPTGNWRFSILRFYWDDEKEPSVECPVGDFFGMGWGVYAPLNSLAICVNPGSAFNCYWTMPFRKKCKITMENVNPEDAMVLYYQVDYTLTDVPADAGYFHAQFRRTHYNETSDYTMIDGVKGKGHYVGTYMAQGVHNNGWWGEGEIKFFMDGDTKYPTICGTGAEDYFCGSYNFDREGQYKEFCTAYAGLHQVIRPDGTYRSQQRFGMYRWHIADPVRFEKDLKITIQDLGWRVGGRYLPQKSDISSVVFWYQGEPHAKFPKFPGWQDLEVN

SEQ ID NO: 13 - protein, 671 aa, Dysgonomonas gadei
GEELLEIRKLPKEFQAVIHSIRFDRLGSIKPQQEIKNIDKGLVHVRLIFDLPAEIKQDDWQLTIQPAFIPDFNWAPHITPGDMNIIDQHVFRSPAMIVTDNKQGLAVIPDINIMKNGTPTRWYMDMIAPENKLVLGMSETLVSGHVIYERKEGAVYPKGRVEFGFYLMKYADKEDLENPFRRPLKFFWTNWGEKLYEEGNPIKGDLEPYVDYTYNWAFNTWKDAVWQEFMLGDKKVGAPVFIVNTTQSPNYPGKVNEREFLSIWNQAWFSSLRSASGLYRYARRKGNKGLLNKALLSKELALAAPMKEGFFYGLIGTEMETVETEGKQYNRSKGWDTYYWGNSNRNPYTWDARKSPFHILDMSWTAILMLRWYEELEKDERLLEYARKYADALLQKQDAKGFFPGWLDLDTLEPMEYLNDSPETSMSVTFLLKLYELTKNEKYRSSALRAIDAVCKEIVPVGRWEDFETYWSCCRLGTPEWIGKKIERNNMYKQCNFSIFWTAEALLDSYRITKNKEYLKLGQRTLDEMLMTQTVWQPPYIYVNAVGGFGVMNADGEWNDSRQSLFAETILQYGKLLNKKEYNERGLSAIRASFSMMYCPENPLAKEQWERVWPFFNEKDYGFTMENYGHDGRTSKDGIGIGEFTIYDWGNGAAAEAYNRIRDKFGIEIFR

SEQ ID NO: 14 - protein, 621 aa, Candidatus Mycosynbacter amalyticus isolate
ARLPQPGGDMGNWGNILNDYLRVAHDEQGNIKAGAIGATQVVDGALPQAKIQNLTTDLSAKYTKPGAGIPKTDLSASVQASLDKADTALQSVPPSAGVATLSGFPLRLAGEKQVSYPIELGKAHTPVANPTGAKKVLLAESWGTAGVLKHIWVASSGGGGVGDFSENGGKIRIYIDDDTTAVVDLSLNDFFAYASQGGEYATRRIGRTKRNTSNGESSAYRYLHMPFQKYLRVEVENTTATDIVFFGSADYTLVNDFAGLGSQQLAYKMYSVEAAAASPYDELTVVDINGSGQVESLWIAVNAASGDTGILEGNVEVYVDGETYPSWRSSGTEDAFNGGWYNVPVGGYPAGRAGDSAQSGLAMTYYRFFVDDPIFFSSHLKVLIHAGQPNQGTITSSTFGLSGFAGVWSNTAGNVNYLAVDTSTTLLNDQFTDTAGSLDTAKWNQVGGVTQGQATGSTITVAYDGSSSGQDVRIARKNAVLPTNYWVETRVRITDATHDGQEVSLIAQGNSPDPYFGSAIHVQLVRFGQNNWVIRARDDFDEVFIRTIGGGRNLTNVWVKLAFKVVGATVTAYWAPDSSPVWQPVGTWVTGKTGSAFGIGTWTAGAEFDYLVVRPLQTYTH

SEQ ID NO: 15 - protein, 664 aa, synthetic construct
  REGION 1..664: note = exo-D-galactofuranosidase: BACFIN_04787
MVNYVPENISASISLKVPGNVAERYSLNLQQLGNNRSSYLLEASESIPVTITQRVADVNGKLRVNVCITALEDIYFNYSQRLSTGYRHDDCQFYMPGFWYRRNLRSPKDAPSFHSSDSWLVREDRLSAPLTGVYSEKEKHFVTVSRIDKFAEEALSTHREGEIILSGKTSLGFTGFENQDGITALSFGFPYREAPKSYIRKLTLAPAVEAFQFLKKGETVLLTWEIQENKADDYSDFIRHAWEYSYDTYGPKPVETPYSITDMKQVLSGFFISSFVEGHPLVYNSGIHLMTENCQSNGQAEVGFIGRVLLNAFNAWEYGHQSDREDLKANSMKVFDSYLKNGFTPVGFFKESVDFDKGYEDPVHSIRRQSEGIYAMLHFLAYEKENGRRHPEWEQKMKNMLDILLRLQQADGSFPRKFRDDFTIVDTSGGSTPSATLPLVMGYKYFKDKRYLASAKQTADYLEKVLISKADYFSSTLDANCEDKEASLYAATATYYLSLITKGDEHRHYADLTKKAAYFALSWYYVWDVPFAQGQMLGDIGLKTRGWGNVSVENNHIDVFVFEFASVLRWLSKEYAEPRFSQFAEVISTSMRQLLPHEGHLCGVAKPGYYPEVVQHTNWDYGKNGKGYYNDIFAPGWTVASLWELFTPGRAEYFLKLEHHHHHH

SEQ ID NO: 16 - protein, 666 aa, synthetic construct
  REGION 1..666: note = exo-D-galactofuranosidase: BACFIN_08811
MGIEYTPANVSASIALKVPGNEAVRYPLEFRKLRKERFDYQLTATESLPVLIYQKVEGGETNKRITLFITATENIYFNYGEQVKSGACHDDCLFYMPGFWYRRNLRSPKEAPSFHTSDSWVVREDRLSTPMTTIFDEKNGKSFSVARIDKFESDALTTHKEGEVILSGTTSIGYTGFENCNGTATLSFGYPYKEAPKTYIRKLTLAPSVEAYQFLGKGESVTLTWELNESAPTDFSDCVKQAWEYSYDLYKPATVETPYTDEVMKEVMSNFFVESFIGDTPTNYYSGVELHTATCNNVDVAEVGFVGRTLLNAFNALEYGESKQRTDLVANANAIFDSYLEHGFSPAGFFNEVVHYKRNFIEPVHSIRRQSEGIYATLYYLNYEKKKGRKHPEWEKRLKGMLDTFLRLQNEDGSFPRKFKDDFSIVDKSGGSTPSATLPLVMGYKYFKDKRYLEAARRSVDYLEKELISKADYFSSTLDANCEDKEASLYASTAAYYLALASKGAERAHYAGLAKKAAYFALSWYYTWDVPFAPGQMLGDIGLKTRGWGNVSVENNHIDVFIFDFADVLRWLSKEYNEPRFADFAAVISSSMRQLLPYEGHQCGIAKKGYYPEVVQHTNWDYGKNGKGYYNHIFAPGWVVASLWELLSPGRPEEFIGRLEHHHHHH

SEQ ID NO: 17 - protein, 319 aa, synthetic construct
  REGION 1..319: note = endo-D-galactofuranase: BACFIN_08810
MQDSEGIYNGASQYRDEIFLADPTILREGDMYYLTGTGNQQQQGFVLLESADLKSWHTVENNEFVLKKDDQTYGTEGFWAPQWFKAQDRYYFLYTANEQTVMASADSWKGPFRQTDIKPIDRSQKNIDPFLFRDDDGKYYLYHVRFGNGNFIWVAEFDLAKGEIIPGTLKKCMESTEEWEATAAYISAPVMEGPTVIKLDGVYYLFYSANDYRSIDYAVGYATSNSPMGPWTKNENSPIIHRSIVGENGSGHGDIFTDKAGNYYYVYHVHASKTNVAPRKTRIIPLHMEKNESGIYDITVNGDEVIEPYQELEHHHHHH

SEQ ID NO: 18 - protein, 462 aa, synthetic construct
  REGION 1..462: note = Endo-D-arabinofuranase: MYCMA_14050
MGSSHHHHHHSSGLVPRGSHMASMTGGQQMGRGSHDPEDEDFNVNEDLSVTERRSHSGLDRLKRAIRRRWLQGILHTRAQELVATDNAIARKLHEIAQDLSNFHLEGPDGNTDFSGHRLKPGESRNLGPVAGTGAPIPGIGSADLGEVIEIPDGKGGKKLVAVFGDSFSTDHVPRPGEAADHYKSVAVEIKGFDQNGKPIWGDVLTGYDGENGRPPLFPTTGLPPAAQNTNTLPAGSIVMRDGTTYMMAAGTNDLHPTGGSWLVKAGDPSQGGWPPEPGTWRAGDKAPSQISGYQAADGTVYIAADSFDRNQQVTMYRVPPGGNVLDRDSWQPMTNTGWGAPGDPAKPLTTTPYGEVSLREVDGRPVLSGLNMGPDPGNGTARVEVRVGSAGDPSSVFAWNSPATVLMQQADPTAPNFVLQNYGGYILPGSTLDNMRVFGSQWVVADGTPYNTQLIEVNPHQ

SEQ ID NO: 19 - protein, 361 aa, synthetic construct
  REGION 1..361: note = Endo-D-arabinofuranase, HMPREF9455_02480
MGSDDVKEDSGTSKNRKHLKVKKVERVARITGTPVDGENLLSSNNTTTMYDVGGTDLGIVWEMNQGKYGIFLGDTFGKGFIPDQNDPADFGDNWRCNVLAFSNDCNLEDGLSIDSMASDENDNARELIPGGKDKSGNGDWTSIPTAAIRANNKDYVHYFNMRNWTGWITNYSGLYKSEDDGKTWGKCTTVHFTSDSNFGQAGYYKKDGYVYMVGTETGRHSNPRLARFNEKDIENQSLYEYWNKDHNEWIRGDESQATNLFEDTVGELSIAYHNKYKCWIITYFCAARYNISIRYAEELTGQWSKPQILATGEEYPQLYGSYIHPVSLDGDDLYFLMSMWRPYNVFLMRAELELEHHHHHH

SEQ ID NO: 20 - protein, 460 aa, synthetic construct
  REGION 1..460: note = Endo-D-arabinofuranase: HMPREF9455_02481
MGSKDGEDEPYIPIDEITVSIAELVPDKETKAVSTTLTFKNIGEIDYLLITKSGGESYSAKIEKPELSASYPFRYMVKKTDPESFRLLLTAYYKDGNKSKEKVLTIDNRWGFFIRKVSRTARITGNPMSGEQFPTPNNTAQKWNVGGTDLGIIWEMNPGKYGIFFGDTFGRDFSPNSAAPGPNGGSWRSNVLAFSDDNDLEDGLTFNSMAVDVSGNAREIIYGGKDASGNGDWTSIPTAAIRANNIDYVHYFNMKNWTGWITNYSGMYKSIDNGQTWAKCENVRFSSNSNFGQGGYFKKDGYVYMIGTQTGRDSNARLARFKEQHIEDQANYEFWNSLTKQWLKGDEEQATNIFNDKVGELSFIYNQKHNKWIISYFNADRYNITMRTATEITGPWSEPYELASGVEYAQLYGSYFHPLSVDGDNLYFLMSMWLPYNVFLMKAELADMGSFDLEHHHHHH

SEQ ID NO: 21 - protein, 361 aa, synthetic construct
  REGION 1..361: note = Endo-D-arabinofuranase, Rv3707c
MAPAPRPILPPLAPGQVLRIGPTAGTGTPTGDYGIGATDLCEFVEFPSQLLQVCGDSFAGQGVGFGGWYAPVALHVDTESIDDPAGVRYTGVTGVGTPLLADPTPPGDSQLPAGVVQINRRNYLMVTTTKDLQPQNSRLVRAEAARGGWQTVSGSRRNAAYQDGRQTQISGYYDPVPTPDSPTGWVYIVADSFTRGEPAVLYRATPESFTDRSRWQGWAGGPDGGWNKPPTPLWPDQLGEMSIRQIDGQTVLSYFNASTGNMEVRVAHHPTSLGAAPVTTVVRHDEWPEPAESLPPPYDNRLAQPYGGYISPGSTIDELRIFVSQWDTRARQNGPYRVIQFAVNPFKPWSDPGSGHHHHHH

SEQ ID NO: 22 - protein, 355 aa, synthetic construct
  REGION 1..355: note = Endo-D-arabinofuranase: MXN_4606
MGSSHHHHHHSSGLVPRGSHMTPSNVTKVSRVTGATPAGELLPNPNQTHTNHDVHGTDLGIVWDKGGGEVFVLFGDTFGAGWCGNGGCGGGWRSNVLARSSDTNLANGLSFSSMIQDGPRHAKEILASRKVNFDEITVIPTAGVTIGSRHYIHYMSIHHWGEAGMWFTNHAGIAYSDDQGQNWVKHPTARWQNTPAWTNNFQMAAFVRNGGFVYMYATPNGRFGNVHLARVPETALLDINGYRYWDGNGWSVSQAAARPVAIGIAGELSVSYHAPLGRFLMTYLNEHRQAVVMRDAATPTGPWSGEKVLASGAVYPGLYNAFIHPWGNTGNSLYFVMSQWTPYNTFLMRATLAHD

SEQ ID NO: 23 - protein, 343 aa, synthetic construct
  REGION 1..343: note = Endo-D-arabinofuranase: GordoniaPhage GMA6_4185
MGSSHHHHHHSSGLVPRGSHMLSDRFGMRATDLGAMIEAPNGKLVAVFGDTWKDRVGGIDWRAPVILIGSKGANGVIQWERAGGSDPNYARQLWAYTHDSPPWTKGGISTVLPSDLLRIGNTLYLHVMVNKGFPTVAWTEIWKSEDSGISWTHLGENSKLSGTAGNGLLQCLSWDYNADDDYVYAVSTGFQRDKGIVLRRFRPTQINNQSLWQVWNYNGTWGWRTNVNPSVITPSGEKWGELTLRKLANGWVLGGLSMHSLGLSYRVLSSPTANLYTTPAKRLIWGCEWPEEDHTVGKVAQTYGGYIVPGSKLGVDGGVDIVVSQWNTTVGWPYKVMQFRGTL

SEQ ID NO: 24 - protein, 362 aa, synthetic construct
  REGION 1..362: note = exo-D-arabinofuranosidase: O3I_017420
MGSSHHHHHHSSGLVPRGSHMTPPRRSELWLLDDTRTRSVSAENPTGAPGGGARATTGTGAHAARLLGPGWKISPSIVLGDGETATLADVSGPGVLRHCWLTTDRAALRELTLRMYWDDAAEPAIDVPLGDFFCNGWDELALVDSEMVVVAPAGGLNSYWPMPFRRRARITLENHCGRSVPVYYQITYGEEDVPGEAGYLHARWSRSAPLGSPAVHTLLDTGPARGRYVGTYLAIRPGAPRWWGEGEMKFYLDGDDEYPTLCGTGTEDYFGGAWNFDMDGKYRTYSTAYLGLPQALPADEIYQAHQRFGMYRWHVHDPICFHRELRVTVQGLGWRDSDTYLPLEDAEIATTVWWYQEVPPSG

SEQ ID NO: 25 - protein, 387 aa, Synthetic construct
  REGION 1..387: note = exo-D-arabinofuranosidase: HMPREF9455_02479
MGQNPVPGTMYELSQMKNGMRNRRISSNDPAGGVLDHLSDIRPGEKRIIADIPGSGIINHIWITMAPEPHVLNRSDVIIRMYWDGNAYPSVESPIGPFFGQGWNERYNYSALPITAGPANGTSMVSYFSMPFAQGARIEIENQSDVNLEKFYFYVDYYETKKLPTDLGRFHAWYNQELTEAAPEGETEWAVIGKQDNNTTGDRNYVFADIKGKGHFVGLNYYVHCPSPIWYGEGDDFWFIDGEEEASLLGTGTEDLFNTSWCPKEAYSHPYFGYPRVNNDVGWLGRTHIYRFFIEDPVFFQKSLKASIEHGHANNLTLDLATVAYWYQSEACPLPPAPSKEVRKLKPFINVPDMHRWRHEWRKNRGEDSKLWGNEMPKQLEHHHHHH

SEQ ID NO: 26 - protein, 379 aa, synthetic construct
  REGION 1..379: note = exo-D-arabinofuranosidase: HMPREF9455_02467
MGFSQEGFNGLGVDMSNLHRLSNAKTRSISPENFTGEKGKGGMADPAMQGDKRNVANAHHAARDLGQTWKVNPYVHIAPGETFTLAEIEGPGAIQHIWMTPTGNWRFSILRFYWDDEKEPSVECPVGDFFGMGWGVYAPLNSLAICVNPGSAFNCYWTMPFRKKCKITMENVNPEDAMVLYYQVDYTLTDVPADAGYFHAQFRRTHYNETSDYTMIDGVKGKGHYVGTYMAQGVHNNGWWGEGEIKFFMDGDTKYPTICGTGAEDYFCGSYNFDREGQYKEFCTAYAGLHQVIRPDGTYRSQQRFGMYRWHIADPVRFEKDLKITIQDLGWRVGGRYLPQKSDISSVVFWYQGEPHAKFPKFPGWQDLEVNLEHHHHHH

SEQ ID NO: 27 - protein, 681 aa, synthetic construct
  REGION 1..681: note = exo-D-arabinofuranosidase: HMPREF9455_02470
MGGEELLEIRKLPKEFQAVIHSIRFDRLGSIKPQQEIKNIDKGLVHVRLIFDLPAEIKQDDWQLTIQPAFIPDFNWAPHITPGDMNIIDQHVFRSPAMIVTDNKQGLAVIPDINIMKNGTPTRWYMDMIAPENKLVLGMSETLVSGHVIYERKEGAVYPKGRVEFGFYLMKYADKEDLENPFRRPLKFFWTNWGEKLYEEGNPIKGDLEPYVDYTYNWAFNTWKDAVWQEFMLGDKKVGAPVFIVNTTQSPNYPGKVNEREFLSIWNQAWFSSLRSASGLYRYARRKGNKGLLNKALLSKELALAAPMKEGFFYGLIGTEMETVETEGKQYNRSKGWDTYYWGNSNRNPYTWDARKSPFHILDMSWTAILMLRWYEELEKDERLLEYARKYADALLQKQDAKGFFPGWLDLDTLEPMEYLNDSPETSMSVTFLLKLYELTKNEKYRSSALRAIDAVCKEIVPVGRWEDFETYWSCCRLGTPEWIGKKIERNNMYKQCNFSIFWTAEALLDSYRITKNKEYLKLGQRTLDEMLMTQTVWQPPYIYVNAVGGFGVMNADGEWNDSRQSLFAETILQYGKLLNKKEYNERGLSAIRASFSMMYCPENPLAKEQWERVWPFFNEKDYGFTMENYGHDGRTSKDGIGIGEFTIYDWGNGAAAEAYNRIRDKFGIEIFRLEHHHHHH

SEQ ID NO: 28 - protein, 631 aa, synthetic construct
  REGION 1..631: note = exo-D-arabinofuranosidase: GII36_05205
MGARLPQPGGDMGNWGNILNDYLRVAHDEQGNIKAGAIGATQVVDGALPQAKIQNLTTDLSAKYTKPGAGIPKTDLSASVQASLDKADTALQSVPPSAGVATLSGFPLRLAGEKQVSYPIELGKAHTPVANPTGAKKVLLAESWGTAGVLKHIWVASSGGGGVGDFSENGGKIRIYIDDDTTAVVDLSLNDFFAYASQGGEYATRRIGRTKRNTSNGESSAYRYLHMPFQKYLRVEVENTTATDIVFFGSADYTLVNDFAGLGSQQLAYKMYSVEAAAASPYDELTVVDINGSGQVESLWIAVNAASGDTGILEGNVEVYVDGETYPSWRSSGTEDAFNGGWYNVPVGGYPAGRAGDSAQSGLAMTYYRFFVDDPIFFSSHLKVLIHAGQPNQGTITSSTFGLSGFAGVWSNTAGNVNYLAVDTSTTLLNDQFTDTAGSLDTAKWNQVGGVTQGQATGSTITVAYDGSSSGQDVRIARKNAVLPTNYWVETRVRITDATHDGQEVSLIAQGNSPDPYFGSAIHVQLVRFGQNNWVIRARDDFDEVFIRTIGGGRNLTNVWVKLAFKVVGATVTAYWAPDSSPVWQPVGTWVTGKTGSAFGIGTWTAGAEFDYLVVRPLQTYTHLEHHHHHH

SEQ ID NO: 29 - protein, 676 aa, Bacteroides finegoldii
MNFKCLFCGALITLYVGVAEAVNYVPENISASISLKVPGNVAERYSLNLQQLGNNRSSYLLEASESIPVTITQRVADVNGKLRVNVCITALEDIYFNYSQRLSTGYRHDDCQFYMPGFWYRRNLRSPKDAPSFHSSDSWLVREDRLSAPLTGVYSEKEKHFVTVSRIDKFAEEALSTHREGEIILSGKTSLGFTGFENQDGITALSFGFPYREAPKSYIRKLTLAPAVEAFQFLKKGETVLLTWEIQENKADDYSDFIRHAWEYSYDTYGPKPVETPYSITDMKQVLSGFFISSFVEGHPLVYNSGIHLMTENCQSNGQAEVGFIGRVLLNAFNAWEYGHQSDREDLKANSMKVFDSYLKNGFTPVGFFKESVDFDKGYEDPVHSIRRQSEGIYAMLHFLAYEKENGRRHPEWEQKMKNMLDILLRLQQADGSFPRKFRDDFTIVDTSGGSTPSATLPLVMGYKYFKDKRYLASAKQTADYLEKVLISKADYFSSTLDANCEDKEASLYAATATYYLSLITKGDEHRHYADLTKKAAYFALSWYYVWDVPFAQGQMLGDIGLKTRGWGNVSVENNHIDVFVFEFASVLRWLSKEYAEPRFSQFAEVISTSMRQLLPHEGHLCGVAKPGYYPEVVQHTNWDYGKNGKGYYNDIFAPGWTVASLWELFTPGRAEYFLK

SEQ ID NO: 30 - protein, 677 aa, Bacteroides finegoldii
MNLRNILSGIALLAGILSAHAIEYTPANVSASIALKVPGNEAVRYPLEFRKLRKERFDYQLTATESLPVLIYQKVEGGETNKRITLFITATENIYFNYGEQVKSGACHDDCLFYMPGFWYRRNLRSPKEAPSFHTSDSWVVREDRLSTPMTTIFDEKNGKSFSVARIDKFESDALTTHKEGEVILSGTTSIGYTGFENCNGTATLSFGYPYKEAPKTYIRKLTLAPSVEAYQFLGKGESVTLTWELNESAPTDFSDCVKQAWEYSYDLYKPATVETPYTDEVMKEVMSNFFVESFIGDTPTNYYSGVELHTATCNNVDVAEVGFVGRTLLNAFNALEYGESKQRTDLVANANAIFDSYLEHGFSPAGFFNEVVHYKRNFIEPVHSIRRQSEGIYATLYYLNYEKKKGRKHPEWEKRLKGMLDTFLRLQNEDGSFPRKFKDDFSIVDKSGGSTPSATLPLVMGYKYFKDKRYLEAARRSVDYLEKELISKADYFSSTLDANCEDKEASLYASTAAYYLALASKGAERAHYAGLAKKAAYFALSWYYTWDVPFAPGQMLGDIGLKTRGWGNVSVENNHIDVFIFDFADVLRWLSKEYNEPRFADFAAVISSSMRQLLPYEGHQCGIAKKGYYPEVVQHTNWDYGKNGKGYYNHIFAPGWVVASLWELLSPGRPEEFIGR

SEQ ID NO: 31 - protein, 329 aa, Bacteroides finegoldii
MRLLHFLIGAMVLIATGCAQDSEGIYNGASQYRDEIFLADPTILREGDMYYLTGTGNQQQQGFVLLESADLKSWHTVENNEFVLKKDDQTYGTEGFWAPQWFKAQDRYYFLYTANEQTVMASADSWKGPFRQTDIKPIDRSQKNIDPFLFRDDDGKYYLYHVRFGNGNFIWVAEFDLAKGEIIPGTLKKCMESTEEWEATAAYISAPVMEGPTVIKLDGVYYLFYSANDYRSIDYAVGYATSNSPMGPWTKNENSPIIHRSIVGENGSGHGDIFTDKAGNYYYVYHVHASKTNVAPRKTRIIPLHMEKNESGIYDITVNGDEVIEPYQE

SEQ ID NO: 32 - protein, 524 aa, Mycobacteroides abscessus
MPTKSEILNWRTDHLGAAAKTWGDQATQLDTAVNGAQGLLDGLQGSGQFVEAFRTRLKSYIDKTRPKVDKLKSAKQVAESSEQQLKGAKDKAVYAVHDPEDEDFNVNEDLSVTERRSHSGLDRLKRAIRRRWLQGILHTRAQELVATDNAIARKLHEIAQDLSNFHLEGPDGNTDFSGHRLKPGESRNLGPVAGTGAPIPGIGSADLGEVIEIPDGKGGKKLVAVFGDSFSTDHVPRPGEAADHYKSVAVEIKGFDQNGKPIWGDVLTGYDGENGRPPLFPTTGLPPAAQNTNTLPAGSIVMRDGTTYMMAAGTNDLHPTGGSWLVKAGDPSQGGWPPEPGTWRAGDKAPSQISGYQAADGTVYIAADSFDRNQQVTMYRVPPGGNVLDRDSWQPMTNTGWGAPGDPAKPLTTTPYGEVSLREVDGRPVLSGLNMGPDPGNGTARVEVRVGSAGDPSSVFAWNSPATVLMQQADPTAPNFVLQNYGGYILPGSTLDNMRVFGSQWVVADGTPYNTQLIEVNPHQ

SEQ ID NO: 33 - protein, 374 aa, Dysgonomonas gadei
MKLSRAIKHIFLLGGFLSPLVVMSDDVKEDSGTSKNRKHLKVKKVERVARITGTPVDGENLLSSNNTTTMYDVGGTDLGIVWEMNQGKYGIFLGDTFGKGFIPDQNDPADFGDNWRCNVLAFSNDCNLEDGLSIDSMASDENDNARELIPGGKDKSGNGDWTSIPTAAIRANNKDYVHYFNMRNWTGWITNYSGLYKSEDDGKTWGKCTTVHFTSDSNFGQAGYYKKDGYVYMVGTETGRHSNPRLARFNEKDIENQSLYEYWNKDHNEWIRGDESQATNLFEDTVGELSIAYHNKYKCWIITYFCAARYNISIRYAEELTGQWSKPQILATGEEYPQLYGSYIHPVSLDGDDLYFLMSMWRPYNVFLMRAELE

SEQ ID NO: 34 - protein, 474 aa, Dysgonomonas gadei
MKTRLFKYIVNIWALAFFTGFYSCSKDGEDEPYIPIDEITVSIAELVPDKETKAVSTTLTFKNIGEIDYLLITKSGGESYSAKIEKPELSASYPFRYMVKKTDPESFRLLLTAYYKDGNKSKEKVLTIDNRWGFFIRKVSRTARITGNPMSGEQFPTPNNTAQKWNVGGTDLGIIWEMNPGKYGIFFGDTFGRDFSPNSAAPGPNGGSWRSNVLAFSDDNDLEDGLTFNSMAVDVSGNAREIIYGGKDASGNGDWTSIPTAAIRANNIDYVHYFNMKNWTGWITNYSGMYKSIDNGQTWAKCENVRFSSNSNFGQGGYFKKDGYVYMIGTQTGRDSNARLARFKEQHIEDQANYEFWNSLTKQWLKGDEEQATNIFNDKVGELSFIYNQKHNKWIISYFNADRYNITMRTATEITGPWSEPYELASGVEYAQLYGSYFHPLSVDGDNLYFLMSMWLPYNVFLMKAELADMGSFD

SEQ ID NO: 35 - protein, 382 aa, Mycobacterium tuberculosis
MRVCSAWVVRCSLVVSFLVPQCVWYARADPPAPAPRPILPPLAPGQVLRIGPTAGTGTPTGDYGIGATDLCEFVEFPSQLLQVCGDSFAGQGVGFGGWYAPVALHVDTESIDDPAGVRYTGVTGVGTPLLADPTPPGDSQLPAGVVQINRRNYLMVTTTKDLQPQNSRLVRAEAARGGWQTVSGSRRNAAYQDGRQTQISGYYDPVPTPDSPTGWVYIVADSFTRGEPAVLYRATPESFTDRSRWQGWAGGPDGGWNKPPTPLWPDQLGEMSIRQIDGQTVLSYFNASTGNMEVRVAHHPTSLGAAPVTTVVRHDEWPEPAESLPPPYDNRLAQPYGGYISPGSTIDELRIFVSQWDTRARQNGPYRVIQFAVNPFKPWSDP

SEQ ID NO: 36 - protein, 656 aa, Myxococcus xanthus
MKRMTTWKRTFLLHATWALLWGTPAAAVTPSNVTKVSRVTGATPAGELLPNPNQTHTNHDVHGTDLGIVWDKGGGEVFVLFGDTFGAGWCGNGGCGGGWRSNVLARSSDTNLANGLSFSSMIQDGPRHAKEILASRKVNFDEITVIPTAGVTIGSRHYIHYMSIHHWGEAGMWFTNHAGIAYSDDQGQNWVKHPTARWQNTPAWTNNFQMAAFVRNGGFVYMYATPNGRFGNVHLARVPETALLDINGYRYWDGNGWSVSQAAARPVAIGIAGELSVSYHAPLGRFLMTYLNEHRQAVVMRDAATPTGPWSGEKVLASGAVYPGLYNAFIHPWGNTGNSLYFVMSQWTPYNTFLMRATLAHDAHSDNLLSEPGFETQAATPVMAPWYLQGSGGVDRALGNARSGQDNGFVNASSGWNALKQSVVVQPYTDYTLRGWVRSSANNTAGYFGARGPGNGPILGEVPFGALGAYTERVVHFNSGPNALIEVYGGLWADNVDTWMQLDDVSLTRGSNLIHQAGFEQQSTNNATSPWYTEGRAGIDRGLGYARTGANNAWARNDTGWNAVKQEVAVTPNTQYTLTGWVKTSTNHADGYFGARLLRSGPILNEVRFAQPLGNYTQLSVSFNSGAQHSVEVFAGMWANTGDTWIQVDDLTLTRN

SEQ ID NO: 37 - protein, 1087 aa, Gordonia Phage
MADRVGIDFSANEIDPAAIKAAGVEAVINYISEARPSAPWMKAVKPMKAEYAKRLRDAGIQIVSNYQYGKTGDATPSDWRGGYEAGKRHGAIALSNHWKAGGTPWRPCYAPCDDNPTMDEVTKYVLPFIKGWAEVWGKEYTGIYCNAQTWDWLKSLGAPVTWFWQHNWDGKAEHPHHPDAHMHQVRIDKDRVGGTGVDWNILLKADYGQWSLSKNPNPIEKFPIVDMIKTSGVGYNAGNSGRLRVYLHSSEGKDWVSTARGTMQYQATSQTGSYHYLIDDNEIIQTIALNNSAWAVLSDNDVSINICLVLSSGATGYGITARENQPKSRAQWLEHEKMLKMLRFLIDHVCRETGIPKTRVDIKGVGQNKRGVSSHNNYTYGSVVLKGFKDGTHWDIPDTFPYSYVLEGSVEPTKPIDPNAWPLPVGYYYGPLDGPEQSISGQFSGDLPEWKDGLGRWQTKVGITASKKWDAQTADAVAKLQKAKGWFTDSNKMKGLLAKGEWDSVITGGWKITDAKPEAPDPTKPFVPTPKPTGRFMPLVEGTYKIGSGYGPRDGGFHRGLDFEAKDGTPFYAMQAGTVLYIGPADGYGQWIVVDSDNAQGAGCVEYGHMWNAFATGLKIGSRVQAGQLLGYVGSNGESTGPHLHVAVHPYEYNPKVYVNPADWLRNAKKHVAPSGSDDNKEVPPVTNPEQPASERAVKVSDLTGDGLSDRFGMRATDLGAMIEAPNGKLVAVFGDTWKDRVGGIDWRAPVILIGSKGANGVIQWERAGGSDPNYARQLWAYTHDSPPWTKGGISTVLPSDLLRIGNTLYLHVMVNKGFPTVAWTEIWKSEDSGISWTHLGENSKLSGTAGNGLLQCLSWDYNADDDYVYAVSTGFQRDKGIVLRRFRPTQINNQSLWQVWNYNGTWGWRTNVNPSVITPSGEKWGELTLRKLANGWVLGGLSMHSLGLSYRVLSSPTANLYTTPAKRLIWGCEWPEEDHTVGKVAQTYGGYIVPGSKLGVDGGVDIVVSQWNTTVGWPYKVMQFRGTLPAVWPDVVRPDASAVLDAADNLTTAMDTYKGGVAATGPEARQIAVVNAELDKLEGIIR

SEQ ID NO: 38 - protein, 342 aa, Nocardia brasiliensis; identical to SEQ ID NO: 10

SEQ ID NO: 39 - protein, 402 aa, Dysgonomonas gadei
MKKKPLKLVLICLLLTLFCIPDLVAQNPVPGTMYELSQMKNGMRNRRISSNDPAGGVLDHLSDIRPGEKRIIADIPGSGIINHIWITMAPEPHVLNRSDVIIRMYWDGNAYPSVESPIGPFFGQGWNERYNYSALPITAGPANGTSMVSYFSMPFAQGARIEIENQSDVNLEKFYFYVDYYETKKLPTDLGRFHAWYNQELTEAAPEGETEWAVIGKQDNNTTGDRNYVFADIKGKGHFVGLNYYVHCPSPIWYGEGDDFWFIDGEEEASLLGTGTEDLFNTSWCPKEAYSHPYFGYPRVNNDVGWLGRTHIYRFFIEDPVFFQKSLKASIEHGHANNLTLDLATVAYWYQSEACPLPPAPSKEVRKLKPFINVPDMHRWRHEWRKNRGEDSKLWGNEMPKQ

SEQ ID NO: 40 - protein, 386 aa, Dysgonomonas gadei
MKKICFLISLFFITQLLFSQEGFNGLGVDMSNLHRLSNAKTRSISPENFTGEKGKGGMADPAMQGDKRNVANAHHAARDLGQTWKVNPYVHIAPGETFTLAEIEGPGAIQHIWMTPTGNWRFSILRFYWDDEKEPSVECPVGDFFGMGWGVYAPLNSLAICVNPGSAFNCYWTMPFRKKCKITMENVNPEDAMVLYYQVDYTLTDVPADAGYFHAQFRRTHYNETSDYTMIDGVKGKGHYVGTYMAQGVHNNGWWGEGEIKFFMDGDTKYPTICGTGAEDYFCGSYNFDREGQYKEFCTAYAGLHQVIRPDGTYRSQQRFGMYRWHIADPVRFEKDLKITIQDLGWRVGGRYLPQKSDISSVVFWYQGEPHAKFPKFPGWQDLEVN

SEQ ID NO: 41 - protein, 691 aa, Dysgonomonas gadei
MLVISFLSLFSSLLFPVRVLGEELLEIRKLPKEFQAVIHSIRFDRLGSIKPQQEIKNIDKGLVHVRLIFDLPAEIKQDDWQLTIQPAFIPDFNWAPHITPGDMNIIDQHVFRSPAMIVTDNKQGLAVIPDINIMKNGTPTRWYMDMIAPENKLVLGMSETLVSGHVIYERKEGAVYPKGRVEFGFYLMKYADKEDLENPFRRPLKFFWTNWGEKLYEEGNPIKGDLEPYVDYTYNWAFNTWKDAVWQEFMLGDKKVGAPVFIVNTTQSPNYPGKVNEREFLSIWNQAWFSSLRSASGLYRYARRKGNKGLLNKALLSKELALAAPMKEGFFYGLIGTEMETVETEGKQYNRSKGWDTYYWGNSNRNPYTWDARKSPFHILDMSWTAILMLRWYEELEKDERLLEYARKYADALLQKQDAKGFFPGWLDLDTLEPMEYLNDSPETSMSVTFLLKLYELTKNEKYRSSALRAIDAVCKEIVPVGRWEDFETYWSCCRLGTPEWIGKKIERNNMYKQCNFSIFWTAEALLDSYRITKNKEYLKLGQRTLDEMLMTQTVWQPPYIYVNAVGGFGVMNADGEWNDSRQSLFAETILQYGKLLNKKEYNERGLSAIRASFSMMYCPENPLAKEQWERVWPFFNEKDYGFTMENYGHDGRTSKDGIGIGEFTIYDWGNGAAAEAYNRIRDKFGIEIFR

SEQ ID NO: 42 - protein, 622 aa, Candidatus Mycosynbacter amalyticus isolate
MARLPQPGGDMGNWGNILNDYLRVAHDEQGNIKAGAIGATQVVDGALPQAKIQNLTTDLSAKYTKPGAGIPKTDLSASVQASLDKADTALQSVPPSAGVATLSGFPLRLAGEKQVSYPIELGKAHTPVANPTGAKKVLLAESWGTAGVLKHIWVASSGGGGVGDFSENGGKIRIYIDDDTTAVVDLSLNDFFAYASQGGEYATRRIGRTKRNTSNGESSAYRYLHMPFQKYLRVEVENTTATDIVFFGSADYTLVNDFAGLGSQQLAYKMYSVEAAAASPYDELTVVDINGSGQVESLWIAVNAASGDTGILEGNVEVYVDGETYPSWRSSGTEDAFNGGWYNVPVGGYPAGRAGDSAQSGLAMTYYRFFVDDPIFFSSHLKVLIHAGQPNQGTITSSTFGLSGFAGVWSNTAGNVNYLAVDTSTTLLNDQFTDTAGSLDTAKWNQVGGVTQGQATGSTITVAYDGSSSGQDVRIARKNAVLPTNYWVETRVRITDATHDGQEVSLIAQGNSPDPYFGSAIHVQLVRFGQNNWVIRARDDFDEVFIRTIGGGRNLTNVWVKLAFKVVGATVTAYWAPDSSPVWQPVGTWVTGKTGSAFGIGTWTAGAEFDYLVVRPLQTYTH

SEQ ID NO: 43 - DNA, 19 bp, synthetic construct
  source 1..19: mol_type = other DNA
  misc_feature 1..19: note = Msmeg_0157, Forward primer
atctgccgctacaaccttc

SEQ ID NO: 44 - DNA, 19 bp, Synthetic construct
  source 1..19: mol_type = other DNA
  misc_feature 1..19: note = Msmeg_0157, Reverse Primer
tgcccttgggattgagatg

SEQ ID NO: 45 - DNA, 20 bp, synthetic construct
  source 1..20: mol_type = other DNA
  misc_feature 1..20: note = Msmeg_0902, Forward primer
cctttccgacgacttcttcc

SEQ ID NO: 46 - DNA, 20 bp, synthetic construct
  source 1..20: mol_type = other DNA
  misc_feature 1..20: note = Msmeg_0902, Reverse primer
ttcctcgacagtgtcagtcc

SEQ ID NO: 47 - DNA, 20 bp, synthetic construct
  source 1..20: mol_type = other DNA
  misc_feature 1..20: note = Msmeg_3943, Forward primer
acgattcgtcctatcacgcc

SEQ ID NO: 48 - DNA, 20 bp, synthetic construct
  source 1..20: mol_type = other DNA
  misc_feature 1..20: note = Msmeg_3943, Reverse primer
acaacaccacgacctttgcc